(19) United States
(12) Patent Application Publication
(10) Pub. No.: US 2025/0319053 A1
(43) Pub. Date: Oct. 16, 2025

(54) INHIBITORS OF THE PEPTIDYL-PROLYL CIS/TRANS ISOMERASE (PIN1), COMBINATIONS AND USES THEREOF

(71) Applicants: DANA-FARBER CANCER INSTITUTE, INC., Boston, MA (US); BETH ISRAEL DEACONESS MEDICAL CENTER, INC., Boston, MA (US); YEDA RESEARCH AND DEVELOPMENT CO. LTD., Rehovot (IL)

(72) Inventors: Kun Ping Lu, Newton, MA (US); Xiao Zhen Zhou, Newton, MA (US); Nathanael S. Gray, Stanford, CA (US); Kazuhiro Koikawa, Brookline, MA (US); Benika Pinch, Brookline, MA (US); Behnam Nabet, Boston, MA (US); Nir London, Rehovot (IL)

(73) Assignees: DANA-FARBER CANCER INSTITUTE, INC., Boston, MA (US); BETH ISRAEL DEACONESS MEDICAL CENTER, INC., Boston, MA (US); YEDA RESEARCH AND DEVELOPMENT CO. LTD., Rehovot (IL)

(21) Appl. No.: 18/572,972

(22) PCT Filed: Jun. 29, 2022
(86) PCT No.: PCT/US2022/035557

§ 371 Date: Dec. 21, 2023

Related U.S. Application Data
(60) Provisional application No. 63/216,952, filed on Jun. 30, 2021.

(51) Int. Cl. A61K 31/203; A61K 31/381; A61K 31/513; A61K 31/7068; A61K 33/36; A61K 39/395; A61P 35/00

(52) U.S. Cl. CPC A61K 31/203; A61K 31/381; A61K 31/513; A61K 31/7068; A61K 33/36; A61K 39/39558; A61P 35/00

(57) ABSTRACT

Disclosed are compounds which inhibit Pin1 activity, methods of making the compounds, pharmaceutical compositions containing the compounds, and methods of using the compounds in combination with immunotherapy and chemotherapy to treat diseases or disorders characterized or mediated by dysregulated Pin1 activity, and wherein the disease comprising cancer. Further disclosed are different type of cancers that can be treated using the methods.

### RELATED APPLICATIONS

This application is a national stage application, filed under 35 U.S.C. § 371, of International Application No. PCT/2022/035557, filed Jun. 29, 2022, which claims the benefit of priority under 35 U.S.C. § 119(e) to U.S. Provisional Application No. 63/216,952, filed Jun. 30, 2021, each of which is incorporated herein by reference in its entirety.

### GOVERNMENT LICENSE RIGHTS

This invention was made with government support under grant numbers R01CA205153 and R01CA167677 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 29, 2022, is named 52095-721001WO_ST25.txt and is 6.06 KB bytes in size.

### BACKGROUND

Pancreatic ductal adenocarcinoma (PDAC) is one of the most aggressive solid malignancies, with near uniform mortality, and is projected to be the second leading cause of cancer deaths by 2030. PDAC is notoriously resistant to chemotherapy, targeted therapies, and immunotherapy (Kleeff et al., Nat Rev Dis Primers 2, 16022 (2016); Brahmer et al., N Engl J Med 366, 2455-2465 (2012)). For example, gemcitabine (GEM) remains a cornerstone of PDAC treatment in all stages of the disease but has only a 23.8% response rate and results in 6.6-month overall survival (OS) in advanced PDAC patients (Amrutkar and Gladhaug, Pancreatic Cancer Chemoresistance to Gemcitabine. Cancers (Basel) 9, 157 (2017); Burris et al., J Clin Oncol 15, 2403-2413 (1997)). The survival rate has been modestly improved by combination treatment with nab-paclitaxel resulting in 8.5-month OS but with increased toxicity (Von Hoff et al., N Engl J Med 369, 1691-1703 (2013)). Even with the more efficacious FOLFIRINOX combination (5-fluorouracil, folinic acid, irinotecan and oxaliplatin), median OS is 11.1 months, but with considerable toxicity (Conroy et al., N Engl J Med 364, 1817-1825 (2011)). Such dismal outcomes have been attributed to inherent intratumor heterogeneity and a desmoplastic and immunosuppressive tumor microenvironment (TME) (Binnewies et al., Nat Med 24, 541-550 (2018); Hanahan and Coussens, Cancer Cell 21, 309-322 (2012); Ho et al., Nat Rev Clin Oncol 17, 527-540 (2020); Hosein et al., Nat Rev Gastroenterol Hepatol 17, 487-505 (2020); Hu et al., Oncol Rep 38, 2069-2077 (2017); McGranahan and Swanton, Cell 168, 613-628 (2017); Neesse et al., Gut (2018); Sahai et al., Nat Rev Cancer 20, 174-186 (2020); Whittle and Hingorani, Gastroenterology 156, 2085-2096 (2019)).

Tumor heterogeneity renders tumors resistant to targeted therapies aimed at blocking individual pathways because multiple pathways are often activated simultaneously and/or rapidly upregulated as a compensatory mechanism (Hanahan and Weinberg, Cell 144, 646674 (2011); Luo et al., Cell 136, 823-837 (2009)). Moreover, individual cancer cells within a tumor, especially in PDAC are highly heterogeneous and continuously evolving (Gerlinger et al., N Engl J Med 366, 883-892 (2012); Hou et al., Cell 148, 873-885 (2012); Shah et al., Nature 486, 395-399 (2012); Biankin et al., PMID: 23103869 (2012); Samuel and Hudson, PMID: 22183185 (2011)). Moreover, the PDAC TME is dominated by dense desmoplasia, and immunosuppressive cell populations (Bayne et al., Cancer Cell 21, 822-835 (2012); Laklai et al., Nat Med 22, 497-505 (2016)), which limit cytotoxic T cell response (Feig et al., Proc Natl Acad Sci USA 110, 20212-20217 (2013); Olive et al., Science 324, 14571461 (2009); Ozdemir et al., Cancer Cell 25, 719-734 (2014); Provenzano et al., Cancer Cell 21, 418-429 (2012)). Cancer-associated fibroblasts (CAFs) play a central role in promoting the desmoplastic and immunosuppressive TME by producing extracellular matrix (ECM) proteins and cytokines, as well as interacting with cancer cells to promote tumor growth and malignancy (Ho et al., Nat Rev Clin Oncol 17, 527-540 (2020); Hosein et al., Nat Rev Gastroenterol Hepatol 17, 487-505 (2020); Neesse et al., Gut (2018); Whittle and Hingorani, Gastroenterology 156, 2085-2096 (2019)). Recent strategies targeting the stroma reduce tumor growth and increase tumor response to chemo- and/or immunotherapy; however, they rarely lead to obvious tumor regression (Carapuca et al., J Pathol 239, 286-296 (2016); Jiang et al., Nat Med 22, 851-860 (2016); Sherman et al., Cell 159, 8093 (2014)). Moreover, some of these approaches even lead to disease acceleration and more aggressive tumors (Ozdemir et al., Cancer Cell 25, 719-734 (2014); Rhim et al., Cancer Cell 25, 735-747 (2014)) and clinical trials have not yet produced promising results, suggesting that targeting the TME might not be sufficient (Ho et al., Nat Rev Clin Oncol 17, 527-540 (2020); Hosein et al., Nat Rev Gastroenterol Hepatol 17, 487-505 (2020); Neesse et al., Gut (2018); Whittle and Hingorani, Gastroenterology 156, 2085-2096 (2019)). Thus, therapies against solid malignancies such as PDAC, are greatly needed.

### SUMMARY OF THE INVENTION

A first aspect of the present invention is directed to a method of treating a disease or disorder mediated by dysregulated Pin1 activity, in a subject, e.g., a human subject, in need thereof, comprising co-administering a therapeutically effective amount of one or more Pin1 inhibitors, or a pharmaceutically acceptable salt or salts thereof, and a therapeutically effective amount of an additional immunotherapy and/or chemotherapy.

Another aspect of the present invention is directed to a method of reducing the activity of Pin1 in a cell, either in vivo or in vitro, comprising co-administering a therapeutically effective amount of one or more Pin1 inhibitors, or a pharmaceutically acceptable salt or salts thereof, and a therapeutically effective amount of an additional immunotherapy and/or chemotherapy.

In some embodiments of any one or more aspects of the invention, the co-administering results in greater therapeutic effect than the effect of the additional immunotherapy and/or chemotherapy when administered alone as a sole active agent, without one or more Pin1 inhibitors. In some embodiments, the one or more Pin1 inhibitors is all-trans retinoic acid (ATRA), arsenic trioxide (ATO), sulfopin, or a combination thereof, or a pharmaceutically acceptable salt or salts thereof. In some embodiments, the one or more Pin1 inhibitors comprises ATRA and ATO (Pin1i−1). In some embodiments, the one or more Pin1 inhibitors comprises sulfopin (Pin1i-2). In some embodiments, the chemotherapy comprises gemcitabine (GEM) or fluorouracil (5-FU). In some embodiments, the immunotherapy is anti-PD-1 or anti-PD-L1. In some embodiments, the co-administering comprises Pin1i−1 and GEM. In some embodiments, the co-administering comprises Pin1i−2 and GEM. In some embodiments, the co-administering comprises Pin1i−1 and 5-FU. In some embodiments, the co-administering comprises Pin1i−2 and 5-FU. In some embodiments, the co-administering comprises Pin1i−1 and anti-PD-1. In some embodiments, the co-administering comprises Pin1i−2 and anti-PD-1. In some embodiments, the co-administering comprises Pin1i−1, anti-PD-1, and GEM. In some embodiments, the co-administering comprises Pin1i−2, anti-PD-1, and GEM. In some embodiments, the method comprises pre-treatment with the one or more Pin1 inhibitors prior to the co-administering.

In some embodiments, the disease is cancer, e.g., pancreatic ductal adenocarcinoma (PDAC), breast cancer, or colorectal cancer. In some embodiments, the cancer is a solid tumor cancer. In some embodiments, the solid tumor cancer is PDAC or breast cancer. In some embodiments, the solid tumor is PDAC. In some embodiments, the solid tumor cancer is breast cancer. In some embodiments, the solid tumor cancer is colorectal cancer. In some embodiments, solid tumor cancer is acute promyelocytic leukemia. In some embodiments, the method comprises pre-treatment with the one or more Pin1 inhibitors prior to the co-administering.

In some embodiments, the cancer or tumor has a desmoplastic and/or an immunosuppressive tumor microenvironment.

Another aspect of the present invention is directed to a pharmaceutical composition, comprising a therapeutically effective amount of one or more Pin1 inhibitors, wherein the one or more Pin1 inhibitor is all-trans retinoic acid (ATRA), arsenic trioxide (ATO), sulfopin, or a combination thereof, or a pharmaceutically acceptable salt thereof, and a therapeutically effective amount of an additional immunotherapy and/or chemotherapy. In some embodiments, the pharmaceutical composition is in the form of a liquid. In some embodiments, the pharmaceutical composition is in the form of a solid. In some embodiments, the pharmaceutical composition is in the form of a tablet or capsule. In some embodiments, the pharmaceutical composition the ATRA is in the form of a slow-release formulation.

### BRIEF DESCRIPTION OF THE DRAWINGS

FIG. 1A is a Sirius Red image and a graph showing collagen deposition. FIG. 1B is an immunofluorescence image and a graph showing CAF proliferation. FIG. 1C is an immunofluorescence image and a graph showing tumor-infiltrating immune cell populations. FIG. 1D is a graph of tumor volume versus treatment days. FIG. 1E is a graph of survival % versus treatment days. FIG. 1F is a set of images showing macroscopic tumors after autopsy. FIG. 1G is a set of images showing microscopic tumors after autopsy. FIG. 1H is a graph of tumor volume showing T-cell depletion rendering GDA tumors aggressive.

FIG. 1A-FIG. 1H show that targeting Pin1 disrupts the desmoplastic and immunosuppressive TME and renders PDAC tumors eradicable by immunochemotherapy in KPC mouse-derived allograft (GDA) Mice. See also FIG. 7A-FIG. 7G, FIG. 8A-FIG. 8N and FIG. 9A-FIG. 9K. FIG. 1A-FIG. 1C show that Pin1 inhibitors disrupt the desmoplastic and immunosuppressive TME in GDA mice. Primary PDAC cells derived from KPC mouse tumors were orthotopically allografted into the pancreas of C57BL/6 wild type mice. When tumor sizes were reached 0.5 cm, as detected by ultrasound or palpation with electronic caliper, mice were treated with two different Pin1 inhibitors, ATRA in 5 mg 21 day slow-releasing formulation+ATO at 2 mg/kg (Pin1i−1), or sulfopin (Pin1i−2) at 40 mg/kg daily for 4 weeks, followed by examining collagen deposition using Sirius Red staining (FIG. 1A), CAF proliferation using double immunofluorescence (IF) for α-smooth muscle actin (αSMA) and Ki67 (FIG. 1), and tumor-infiltrating immune cell populations (CD8α+ T-cells, FOXP3+ Tregs, Ly6G+ CD11b+ Myeloid cells) using IF (FIG. 1C). FIG. 1D-FIG. 1G show that Pin1 inhibitors render PDAC eradicable by immunochemotherapy in GDA mice. Overt tumor-bearing (>0.5 cm) GDA mice were treated with Pin1i, low dose GEM at 10 mg/kg (i.p., weekly)+αPD1 at 200 pg/mouse (G+P), Pin1 inhibitor (Pin1i)+αPD1, or Pin1i+G+P for up to 120 days and monitored for tumor growth (FIG. 1D), and overall survival using Kaplan-Meier survival analysis (FIG. 1E) for up to 365 days, as well as examining macroscopic tumors (FIG. 1F) or microscopic tumors (FIG. 1G) after autopsy (n=8-16). Median survival; vehicle 33 days, G+P 38 days, Pin1i−1 43.5 days, and Pin1i−1+αPD1 72.4 days (FIG. 1E). CR, complete remission. FIG. 1H shows that CD8α+ T-cell depletion renders GDA tumors aggressive. Overt tumor-bearing GDA mice were treated with vehicle, αPD1+Pin1i−2, anti-CD8α (αCD8α), anti-NK1.1 (αNK1.1), or their combination for 3 weeks, followed by assaying tumor growth and volume. Scale bars; 200 μm (FIG. 1A), 50 μm (FIG. 1), and 100 μm (FIG. 1C), 2000 and 100 μm (high magnification) (FIG. 1G). Error bars represent mean±s.d. (FIG. 1A); *p<0.05, **p<0.001, ***p=0.0001, ****p<0.0001; n.s., not significant; by unpaired two-sided Student's t test (FIG. 1A-FIG. 1C), long-rank test (FIG. 1E), or one-way analysis of variance (ANOVA) for multiple comparisons (FIG. 1H).

FIG. 2A is a set of images showing Pin1 overexpression in PDAC tissues and normal control. FIG. 2B is a set of images showing the relationship of Pin1 overexpression at different stages of PDAC cancer progression in cancer cells and CAFs. FIG. 2C is a set of tissue-based immunofluorescence images for Pin1 overexpression in various subtypes of CAFs. FIG. 2D is a graph showing the quantification of Pin1expression for various cell types. FIG. 2E is a graph of survival % versus time in cancer cells and CAFs for Pin1 overexpression. FIG. 2F is a graph of survival % versus time for human PDAC patients. FIG. 2G is a set of IHC and Sirius Red images of normal tissue and cancer tissue for Pin1 overexpression together with a graph of collagen area % for cancer cells and CAFs for Pin1 overexpression. FIG. 2H is a set of IHC images and a graph of CD8 area % for cancer cells and CAFs for Pin1 overexpression. FIG. 2I is a set of IHC images and a graph of CD163 area % for cancer cells and CAFs for Pin1 overexpression.

FIG. 2A-FIG. 2I show that Pin1 is overexpressed in cancer cells and CAFs in human PDAC, and strongly correlates with the desmoplastic and immunosuppressive TME, and poor patient survival. FIG. 2A-FIG. 2D show that Pin1 is overexpressed in cancer cells and CAFs and correlates with PDAC progression. Representative images show Pin1 overexpression in PDAC tissues, as compared with normal control, the relationship of Pin1 overexpression at different stages of PDAC cancer progression (FIG. 2A). Pin1 overexpression in both cancer cells (black arrow; Pin1+CK19+αSMA− cells) and CAFs (white arrow; Pin1+ CK19− αSMA+ cells) were assayed by immunohistochemistry (IHC) (FIG. 2B), and Pin1 overexpression in various subtypes of CAFs by tissue-based cyclic immunofluorescence (t-CyCIF): Pin1 (white), pan-cytokeratin (Pan-CK), αSMA, human leukocyte antigen (HLA) class II histocompatibility antigen, DP beta 1 (HLA-DPB1), CD44 and CD45 (FIG. 2C); quantification of Pin1 expression (single cell measurements by indicated markers; median, quartiles and total data range for indicated cell types) (FIG. 2D). White arrows indicate Pin1+ and DPB1+ or CD44+ CAFs or Pin1+ and CD45+ cells (FIG. 2C). CAFs were defined as double negatives for Pan-CK and CD45 and subtyped by k-means clustering based on normalized values of αSMA, CD44 and DPB1 (FIG. 2D). FIG. 2E-FIG. 2F show that Pin1 overexpression in cancer cells or CAFs correlates with overall survival. PDAC tissues were classified into Pin1-High or Pin1-Low groups based on IHC intensity and areas, followed by examining patient overall survival using Kaplan-Meier survival analysis. Median survival rates for Pin1 high and low in cancer cells were 18.0 and 45.3 months (left), and for Pin1 high and low in CAFs were 16.4 and 44.0 months (right), respectively. Pin1 overexpression both in cancer cells and CAFs strongly correlates with poor overall survival in human PDAC patients, with median survival rates in cancer high and CAF high group (n=73) and in cancer low and CAF low group (n=38) being 16.0 and 60.0 months, respectively (FIG. 2F). FIG. 2G-FIG. 2I show that Pin1 overexpression correlates with the desmoplastic and immunosuppressive TME. Representative images of PDAC with Pin1 IHC and Sirius red staining. Pin1 overexpression in CAFs, but not in cancer cells, was correlated with tumor fibrosis (Sirius red staining, n=46) (FIG. 2G). Representative images of PDAC with Pin1 and CD8 IHC staining (FIG. 2H), and Pin1 and CD163 IHC staining (FIG. 2I), followed by examining CD8 or CD163 positive cells area per field. (n=45) (FIG. 2H, FIG. 2I). Pin1 overexpression in CAFs more strongly correlates with immunosuppressive TME (CD8+ T-cells and CD163+ tumor associated macrophages, IHC staining, n=45) than that in cancer cells. Scale bars, 300 and 50 μm (inset) (FIG. 2A), 1000 and 100 μm (high magnification) (FIG. 2B), 100 μm and 50 μm (FIG. 2C) and 300 μm (FIG. 2G-FIG. 2I). *p<0.05, **p<0.001, ***p=0.0001, ****p<0.0001; n.s., not significant; by unpaired two-sided Student's t test (FIG. 2G-FIG. 2I), log-rank test (FIG. 2E, FIG. 2F).

FIG. 3A is an image of an immunoblot for various concentrations of Pin1 inhibitors. FIG. 3B is a set of two graphs of cell growth % versus concentration in CAF1 and CAF2 for various concentrations of Pin1 inhibitors. FIG. 3C is a set of phase contrast images for quiescent phenotype using BODIPY. FIG. 3D is a graph of lipid droplets/CAF corresponding to the images shown in FIG. 3C. FIG. 3E is an image showing cytokine production in primary human CAFs for Pin1 KD versus Pin1i−1. FIG. 3F is a cartoon showing PDAC organoids seeded to the top of Matrigel followed by co-culturing for 10 days. FIG. 3G is a set of images showing the examination of organoid growth and invasion for the Pin1 KD or control CAFs seed according to FIG. 3F. FIG. 3H is a graph of organoid area corresponding to the images shown in FIG. 3G. FIG. 3I is set of images and a graph of tumor volume showing tumor growth for human PDAC organoids in PDOX mice. FIG. 3J is a Sirius Red image and a graph showing collagen area % showing fibrosis for human PDAC organoids in PDOX mice.

FIG. 3A-FIG. 3J show that Pin1 promotes oncogenic signaling pathways, CAF activation and crosstalk with cancer cells to enhance tumor growth and malignancy in organoids and patient-derived orthotopic xenografts (PDOX). FIG. 3A-FIG. 3E show that Pin1i, knockdown (KD) or knockout (KO) reduce Pin1 and its substrate oncoproteins, suppress cell growth, induce quiescent phenotype, and reduce cytokine production in primary human CAFs. Primary CAFs derived from two different human PDAC patients (CAF1 or CAF2) were treated with Pin1i−1 or Pin1i−2 for 72 hrs or subjected to stable Pin1 KD or KO, followed by examining Pin1 and its substrate oncoproteins using immunoblotting (IB), as in FIG. 3A, cell growth using proliferation assay, as in FIG. 3B, quiescent phenotype as measured by lipid droplets/CAF cell using BODIPY as in FIG. 3C and FIG. 3D, and cytokine production using cytokine array, as in FIG. 3E. ATRA and ATO were used in 10:1 ratio as Pin1i−1 and only ATRA concentrations were shown to simplify labeling as in FIG. 3A and in FIG. 3B. CAFs were treated with Pin1i−1 at 10 μM (ATRA 10 at μM+ATO at 1 μM) or Pin1i−2 at 5 μM for 72 hrs, as in FIG. 3C, FIG. 3D, and FIG. 3E. FIG. 3F-FIG. 3H show that Pin1 KD CAFs fail to promote PDAC growth and invasion in the human organoid indirect co-cultured model. Primary human PDAC organoids were derived from two different human PDAC patients (PDAC1 and PDAC2). Pin1 KD or control CAFs were seeded to the top of Matrigel that contained pre-formed human PDAC organoids and co-cultured for 10 days, as shown in FIG. 3F, followed by examining organoid growth and invasion into Matrigel using microscopic imaging analysis, as shown in FIG. 3G and FIG. 3H. FIG. 3I and FIG. 3J show that Pin1 KO CAFs fail to promote tumor growth and fibrosis of human PDAC organoids in PDOX mice. Human PDAC organoids were orthotopically transplanted alone or with Pin1 KO CAFs or CRISPR control CAFs into the pancreas of NOD skid gamma (NSG) mice for 5 weeks, followed by examining tumor growth as in FIG. 3l, and fibrosis using Sirius red staining, as in FIG. 3J (n=5). See also FIG. 10. Scale bars, 100 (upper panels) and 50 μm (lower panels), FIG. 3C, 100 μm, FIG. 3G, and 200 μm, FIG. 3J. Error bars represent mean±s.d.; *p<0.05, **p<0.001, ***p=0.0001, ****p<0.0001; n.s., not significant; by one-way ANOVA for multiple comparisons.

FIG. 4A is a set of images showing apoptosis in PDAC organoids for Pin1 inhibitors with and without GEM. FIG. 4B is a graph of apoptotic organoid cell % corresponding to the images from FIG. 4A. FIG. 4C is an immunoblot for various concentrations of Pin1 inhibitors and Pin1 and its substrate oncoproteins, and PD-L1 and ENT1 in primary human PDAC cells. FIG. 4D is an immunoblot showing Pin1 KD or KO for PD-L1, ENT1 and Pin1. FIG. 4E is a set of IF images and a graph of PD-L1 expression fold-change at the cell surface of human PDAC cancer cells. FIG. 4F is a set of IF images and a graph of ENT1 expression fold-change at the cell surface of human PDAC cancer cells. FIG. 4G is a set of IHC images showing PD-L1 expression for Pin1i−2. FIG. 4H is a set of IHC images and a graph showing fold-change in expression of PD-L1. FIG. 4I is a set of IHC images showing PD-L1 expression for Pin1i−1. FIG. 4J is a set of IHC images and a graph showing fold-change in expression of ENT1.

FIG. 4A-FIG. 4J show that Pin1 promotes oncogenic signaling pathways and reduces the expression of PD-L1 and equilibrative nucleoside transporter 1 (ENT1) at the cell surface of PDAC cells. FIG. 4A and FIG. 4B show that Pin1 inhibitors greatly potentiate the ability of GEM to induce PDAC organoid apoptosis. Established human PDAC organoids were pre-treated with control (DMSO) or Pin1i−1 at 10 μM (ATRA at 10 M+ATO at 1 μM) or Pin1i−2 at 5 μM for 72 hrs, and then treated with control (PBS) or GEM at 25 nM for 24 hrs, followed by examining organoid apoptosis using caspase 3/7 fluorescence green reagent. FIG. 4C shows that Pin1 inhibitors reduce Pin1 and its substrate oncoproteins including Kras signaling proteins and increase PD-L1 and ENT1 in primary human PDAC cells. Human PDAC2 cells were treated with Pin1i−1 or Pin1i−2 at various concentrations for 72 hrs, followed by immunoblotting for different proteins indicated. ATRA and ATO were used in 10:1 ratio as Pin1i−1 and only ATRA concentrations are shown. FIG. 4D shows that Pin1 KD or KO increases PD-L1 and ENT1 expression in primary human PDAC cells. Human PDAC cells were subjected to Pin1 KD or KO, followed by IB for different proteins indicated. FIG. 4E and FIG. 4F show that Pin1 inhibitors increase the expression of PD-L1 or ENT1 notably at the cell surface of human PDAC cancer cells. Human PDAC2 cells were treated with control (DMSO), Pin1i−1 at 10 M, or Pin1i−2 at 5 μM for 72 hrs, followed by IF for Pin1, PD-L1, and DAPI, as in FIG. 4E, or Pin1, ENT1, and DAPI, as in FIG. 4F. White arrows point to PD-L1 in FIG. 4E and ENT 1 in FIG. 4F at the cell surface. FIG. 4G-FIG. 4J show that Pin1 inhibitors increase the expression of PD-L1 or ENT1 notably at the cell surface in GDA mice. GDA mice were treated with vehicle, Pin1i−1 or Pin1i−2 for 4 weeks, followed by IHC for PD-L1, as in FIG. 4G, or for ENT1, as in FIG. 4I, IF for Pin1, PD-L1, and DAPI, as in FIG. 4H, or for Pin1, ENT1, and DAPI as in FIG. 4J, (n=5). White arrows point to PD-L1 in FIG. 4H and ENT1 in FIG. 4J at the cell surface. See also FIG. 11 and FIG. 12. Scale bars, 100 μm for FIG. 4A, 50 μm for FIG. 4E and FIG. 4F, 500 μm and 100 μm (right panels) for FIG. 4G and FIGS. 4I, and 50 μm and 12.5 μm (inset) for FIG. 4H and FIG. 4J. Error bars represent mean±s.d.; *p<0.05, **p<0.001, ***p=0.0001, ****p<0.0001; n.s., not significant; by unpaired two-sided Student's t test for FIG. 4H and FIG. 4J or one-way ANOVA for multiple comparisons for FIG. 4B, FIG. 4E, and FIG. 4F.

FIG. 5A is an immunoblot for various concentrations of Pin1 inhibitors and Pin1, HIP1R and CMTM6 levels. FIG. 5B is a graph of pHIP1R level (fold) at various concentrations of Pin1 inhibitors corresponding to the immunoblot shown in FIG. 5A. FIG. 5C is an immunoblot for Pin1 KO and KD and Pin1, HIP1R and CMTM6 levels. FIG. 5D is a graph of pHIP1R level (fold) for Pin1 KO and KD corresponding to the immunoblot shown in FIG. 5A. FIG. 5E is an immunoblot showing co-IP of Pin1 and phosphorylated HIP1R in PDAC cells. FIG. 5F is an immunoblot showing PD-L1 and ENT1 levels due to HIP1R KD. FIG. 5G is an immunoblot showing PD-L1 and ENT1 levels due to S929A mutation. FIG. 5H is an immunoblot showing that the S929A mutation in HIP1R impairs binding to endogenous Pin1 in PDAC cells using the Pin1 antibody. FIG. 5I is an immunoblot showing that the S929A mutation in HIP1R impairs binding to endogenous Pin1 in PDAC cells using the Flag antibody. FIG. 5J is an immunoblot showing that the S929A mutation in HIP1R impairs its binding to actin in PDAC cells. FIG. 5K is a set of IF images and a graph showing co-localization for Pin1i−1 and HIP1R. FIG. 5L is a set of IF images and a graph showing co-localization for Pin1i−1 and LAMP. FIG. 5M is a set of IF images and a graph showing co-localization for Pin1i−1 and PD-L1. FIG. 5N is a set of IF images and a graph showing co-localization for Pin1i−1 and ENT1.

FIG. 5A-FIG. 5N show that Pin1 promotes the endocytosis and lysosomal degradation of PD-L1 and ENT1 by acting on the pS929-Pro motif in HIP1R. FIG. 5A-FIG. 5D show that Pin1 inhibitors, KD or KO increase phosphorylated HIP1R levels, with little effect on CMTM6 in primary human PDAC cells. Human PDAC2 cells were treated with Pin1i−1 or Pin1i−2 at various concentrations for 72 hrs as shown in FIG. 5A and FIG. 5B or subjected to Pin1 KD or KO as shown in FIG. 5C and FIG. 5D, followed by IB for the different proteins indicated, as shown in FIG. 5A and FIG. 5C, and quantifying phosphorylated HIP1R, as shown in FIG. 5B and FIG. 5D. A white arrow points to phosphorylated HIP1R and a black arrow to non-phosphorylated HIP1R. ATRA and ATO were used in 10:1 ratio as Pin1i−1 and only ATRA concentrations are shown in FIG. 5A and FIG. 5B. FIG. 5E shows Pin1 Co-IPs with phosphorylated HIP1R, but not CMTM6 in PDAC cells. PDAC2 cell lysates were incubated with calf intestinal phosphatase (+CIP) or CIP plus phosphatase inhibitors (-CIP), followed by IB directly (input) or after immunoprecipitation (IP) with Pin1 antibodies. FIG. 5F shows that HIP1R KD increases PD-L1 and ENT1 levels in PDAC cells. HIP1R in PDAC2 cells was stably knocked down using shRNA or vector control, followed by IB for PD-L1 and ENT1. FIG. 5G shows that the S929A mutation in HIP1R increases PD-L1 and ENT1 levels in PDAC cells. Flag-HIP1R wild type (WT) or its S922A mutant was stably expressed in PDAC2 cells, followed by IB. FIG. 5H and FIG. 5I show that the S929A mutation in HIP1R impairs binding to endogenous Pin1 in PDAC cells shown by reciprocal co-immunoprecipitation (Co-IP). PDAC2 cells stably expressing Flag-HIP1R WT or S929A mutant were subjected to IB directly (input) or after IP with Pin1 antibody as in FIG. 5G or Flag antibody as in FIG. 5H. FIG. 5J shows that the S929A mutation in HIP1R impairs its binding to actin in PDAC cells. PDAC2 cells stably expressing Flag-HIP1R WT or its S929A mutant were subjected to IB directly (input) or after IP with Flag antibody. FIG. 5K-FIG. 5N show that Pin1, PD-L1 and ENT1 co-localize with HIP1R, but not HIP1R S929A, at perinuclear lysosomes. PDAC2 cells were treated with control (DMSO) or Pin1i−1 at 10 μM for 72 hrs, and subjected to IF for Pin1, HIP1R, and DAPI as shown in FIG. 5K, and HIP1R WT or S929A stably transfected PDAC2 cells were subjected to IF for HIP1R, LAMP1, and DAPI as shown in FIG. 5L, HIP1R, PD-L1, and DAPI as shown in FIG. 5M, for HIP1R, ENT1, and DAPI as shown in FIG. 5N, followed by assaying co-localization of Pin1-HIP1R as shown in FIG. 5K, HIP1R-LAMP1 as shown in FIG. 5L, HIP1R-PD-L1 as shown in FIG. 5M, and HIP1R-ENT1 as shown in FIG. 5N using Image J program, with R value being Pearson's coefficient for co-localization. See also FIG. 12. Scale bars 50 μm and 5 μm (inset) in FIG. 5K, and 20 μm and 5 μm (inset) in FIG. 5L-FIG. 5N. Error bars represent mean±s.d.; *p<0.05, **p<0.001, ***p=0.0001, ****p<0.0001; n.s., not significant; by unpaired two-sided Student's t test (FIG. 5K-FIG. 5N), or one-way ANOVA for multiple comparisons in FIG. 5B and FIG. 5D.

FIG. 6A is an immunoblot showing the Pin1 inhibitor time-dependent induction of PD-L1 and ENT1 in human PDAC organoids. FIG. 6B is a flow cartoon showing the treatment of established organoids with Pin1 inhibitors followed by either GEM or 5-FU or anti-PD-1, anti-PD-L1 or GEM plus anti-PD-1. FIG. 6C is a set of images showing human PDAC organoid apoptosis upon treatment with GEM, Pin1i−1, and GEM plus Pin1i−1. FIG. 6D is a graph of apoptotic organoid cell % versus time for GEM, Pin1i−1, and GEM plus Pin1i−1. FIG. 6E is a graph showing the synergy score as determined by GEM concentration versus Pin1i−1 concentration. FIG. 6F is a set of images showing human PDAC organoid apoptosis upon treatment with anti-PD-1, Pin1i−1, and anti-PD-1plus Pin1i−1. FIG. 6G is a graph of apoptotic organoid cell % versus time for anti-PD-1, Pin1i−1, and anti-PD-1plus Pin1i−1. FIG. 6H is a graph showing the synergy score as determined by GEM concentration versus Pin1i−1 concentration. FIG. 6I is a set of images showing PDAC organoid apoptosis at different times for GEM plus Pin1i−1, anti-PD-1 plus Pin1i−1, and GEM plus anti-PD-1 plus Pin1i−1. FIG. 6J is a graph of apoptotic organoid cell % versus time corresponding to the images shown in FIG. 6I. FIG. 6K is a graph of synergy scores for Pin1i, GEM and/or anti-PD-1. FIG. 6L is a graph of apoptotic organoid cell % showing that KPC tumor-bearing mouse derived CD8+ T-cells induce KPC mouse organoid apoptosis. FIG. 6M is a set of images showing that Pin1i−2 synergizes with anti-PD-1 to induce the KPC organoid apoptosis. FIG. 6N is a graph of apoptotic organoid cell % showing that Pin1i−2 synergizes with anti-PD-1 to induce the KPC organoid apoptosis.

FIG. 6A-FIG. 6N show that targeting Pin1 synergizes with immunochemotherapy to induce PDAC organoid apoptosis. FIG. 6A shows that Pin1 inhibitors time-dependently induce PD-L1 and ENT1 in human PDAC organoids. Established primary human PDAC2 organoids were treated with control (DMSO), Pin1i−1 at 10 μM or Pin1i−2 at 5 μM for 3 or 7 days, followed by IB. As shown in FIG. 6B, primary human or KPC mouse PDAC organoids were pre-treated with control (DMSO) or Pin1i for 3 days, followed by i) treating with control (PBS) or GEM, or control (DMSO) or 5-FU, or ii) co-culturing with human PBMCs or mouse derived CD8+ T-cells that had been activated by CD3/28 beads and IL-2, and treating with control (IgG), αPD1 or αPD-L1, or control (PBS+IgG) or GEM+αPD1. Apoptosis of PDAC organoids was measured using caspase 3/7 green fluorescent reagent and time-lapse live imaging. Two different patient derived PDAC organoids (PDAC1 and PDAC2 organoid) were tested, and consistent results were obtained.

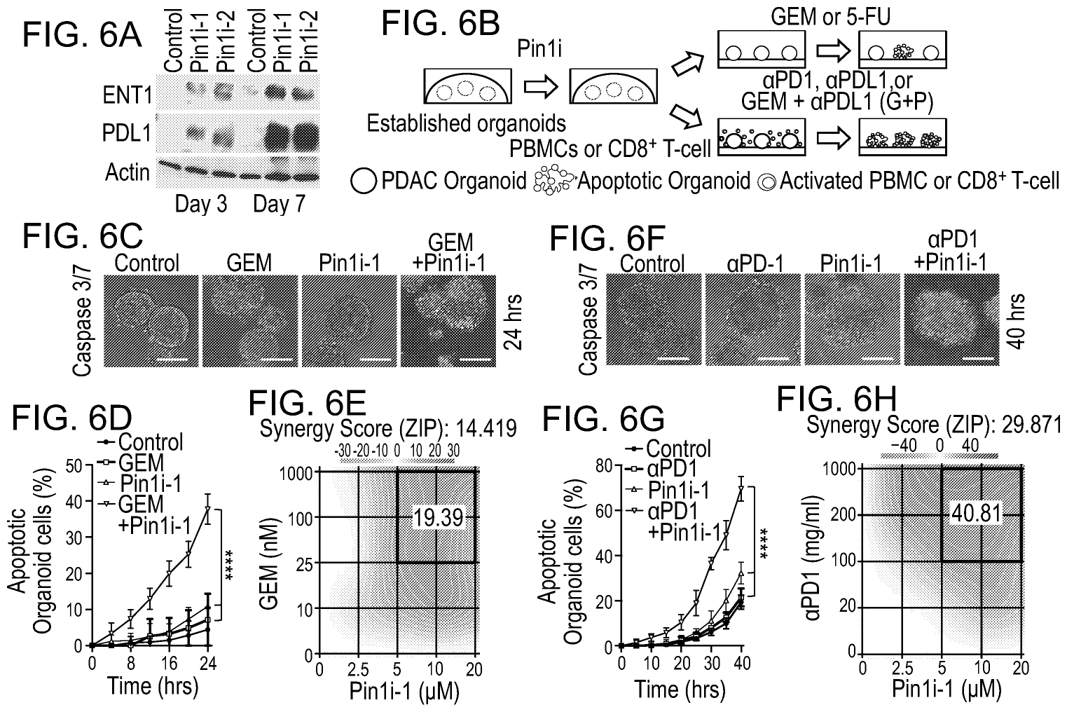

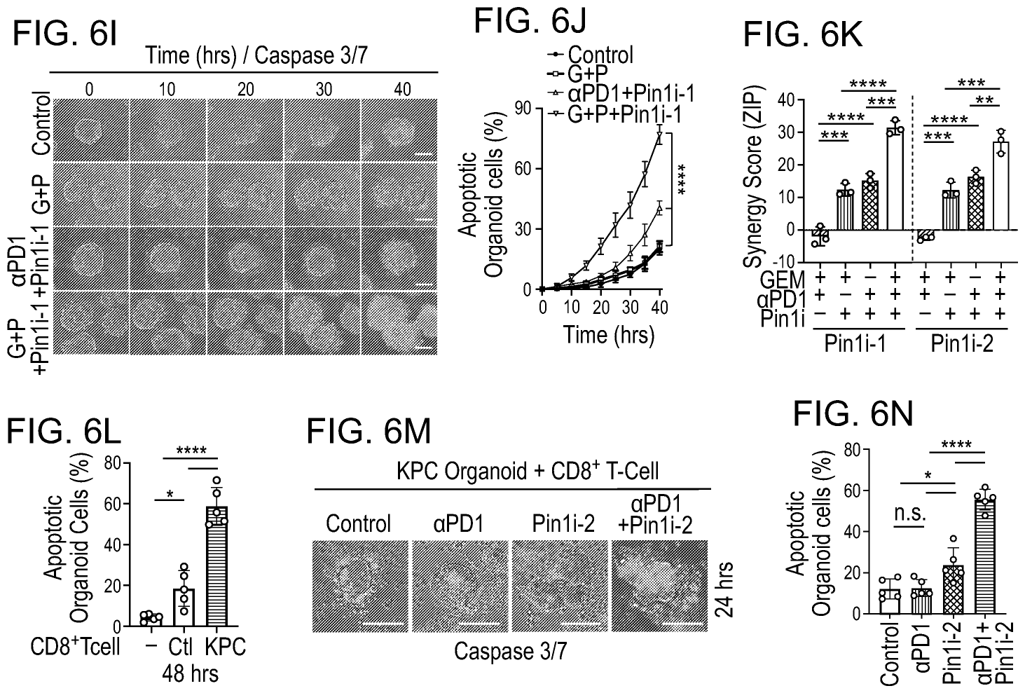

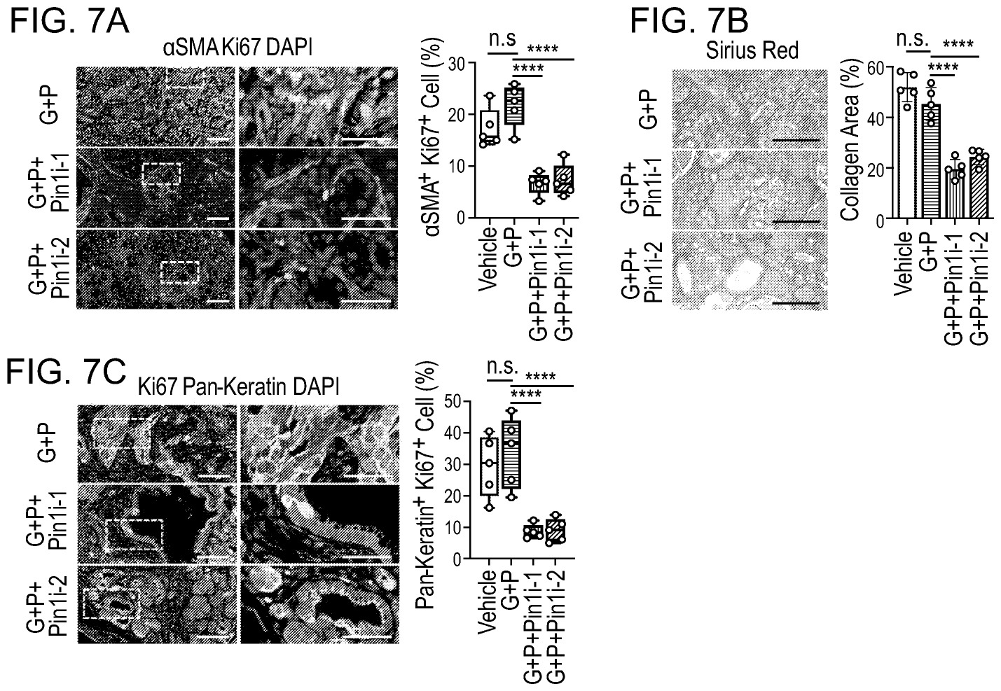

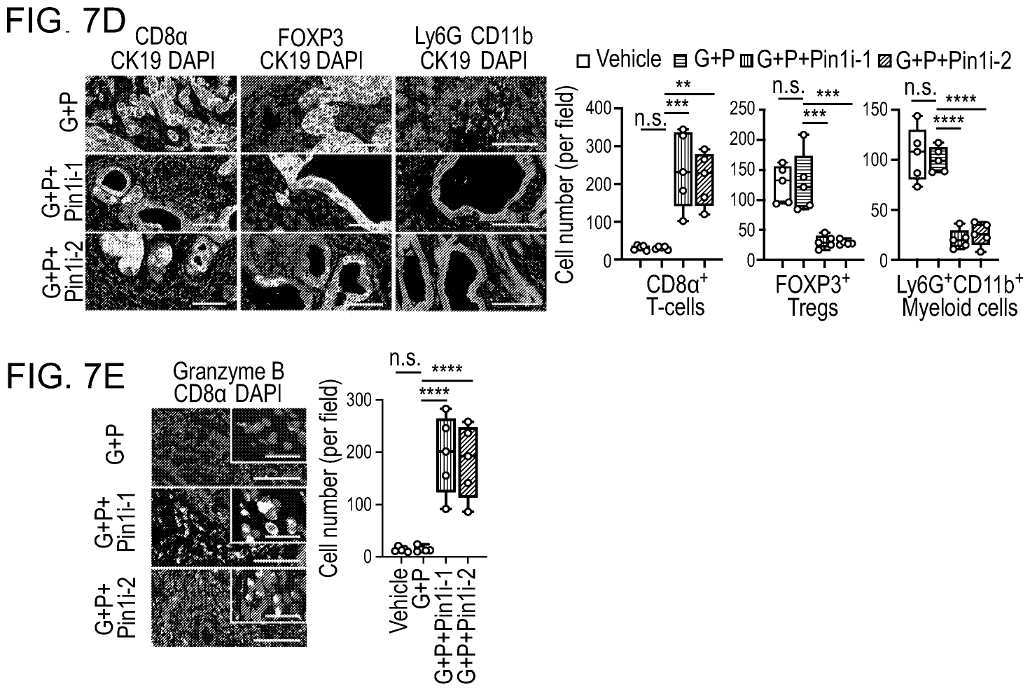

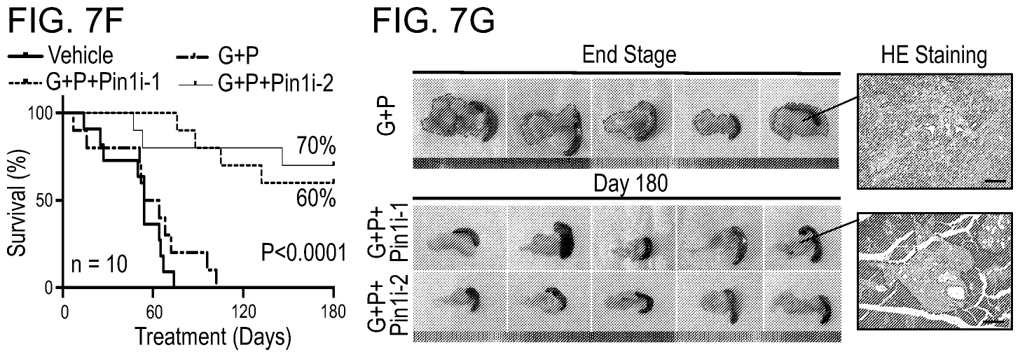

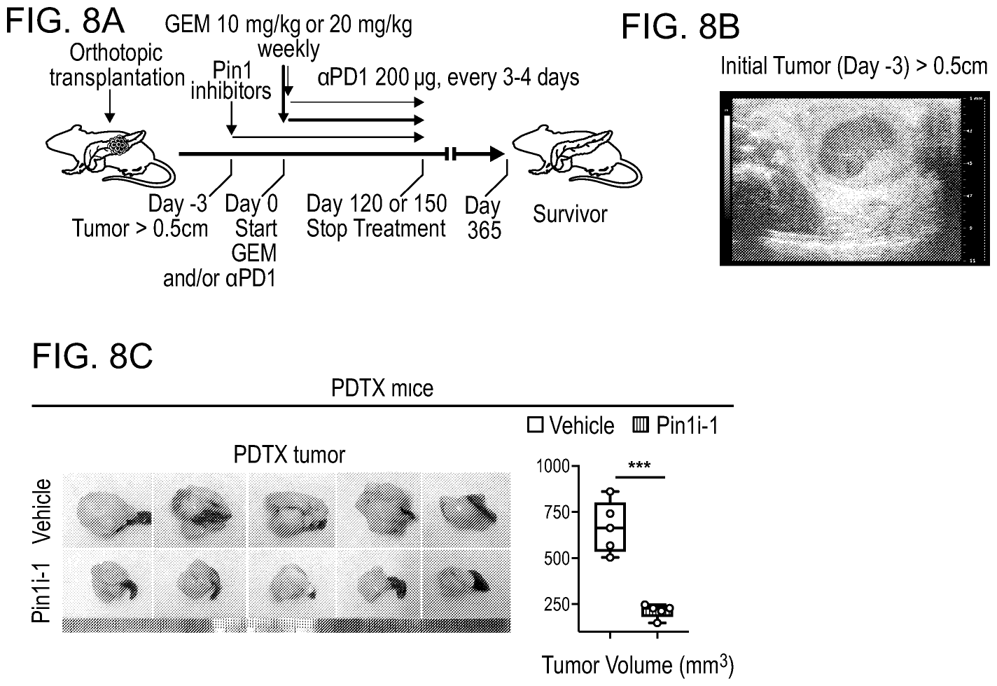

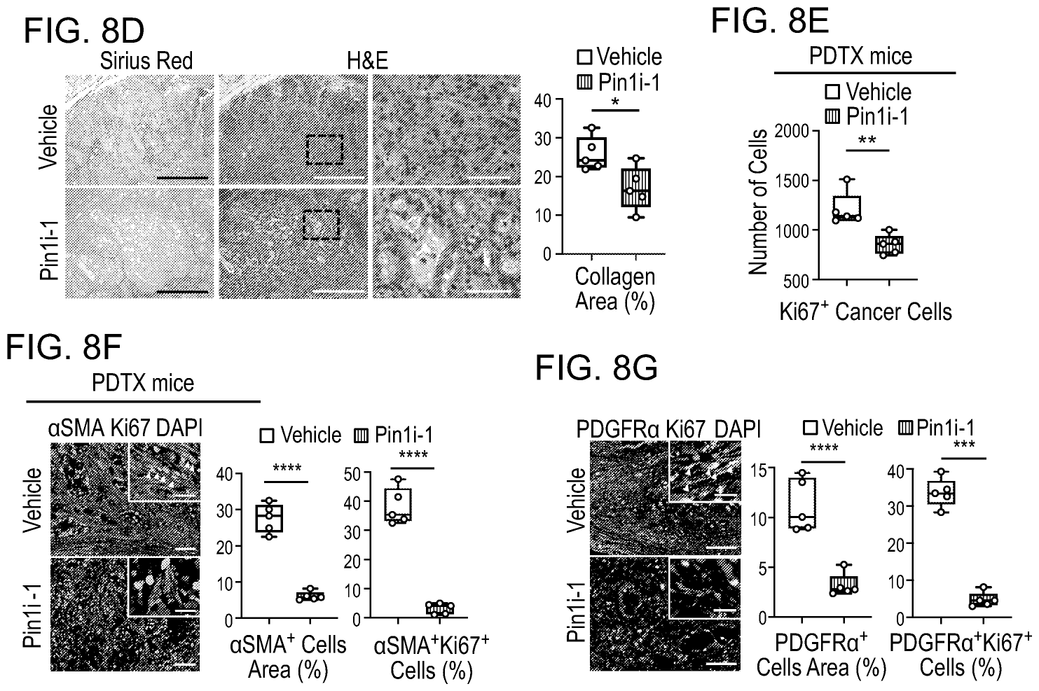

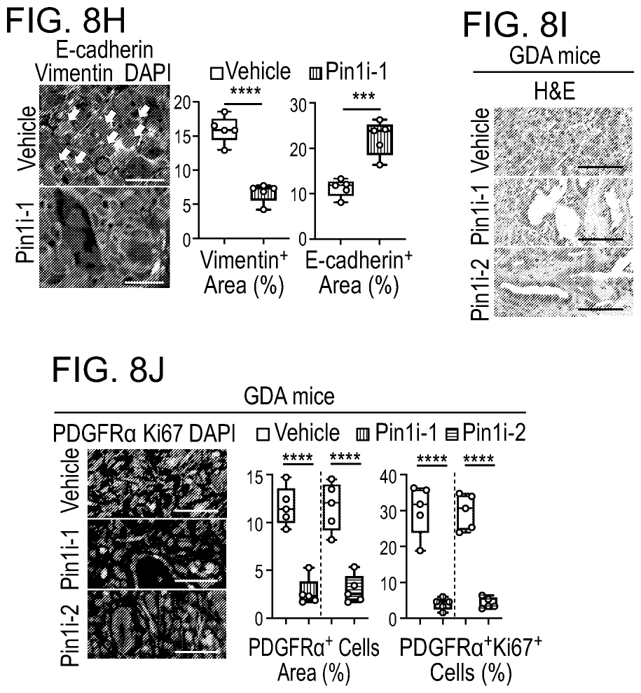

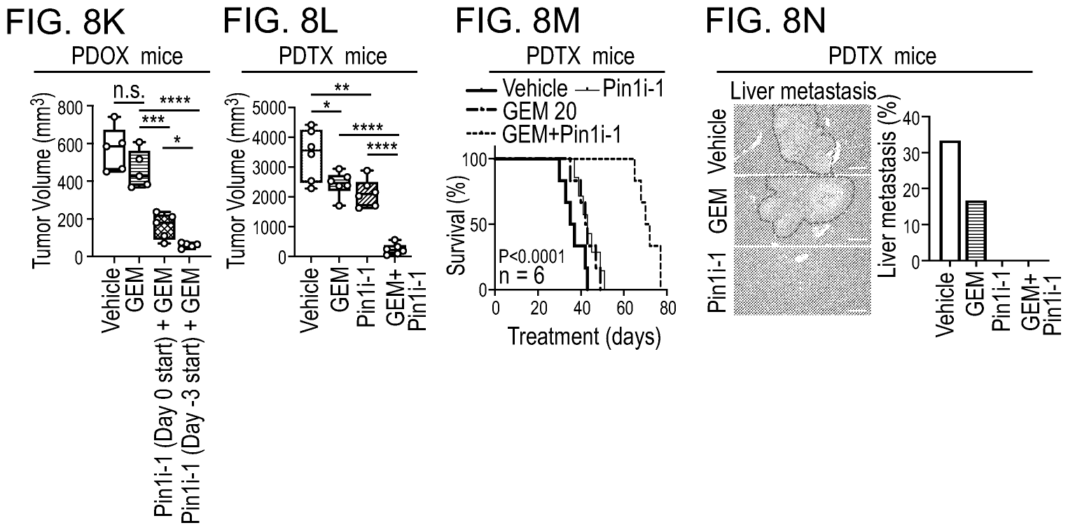

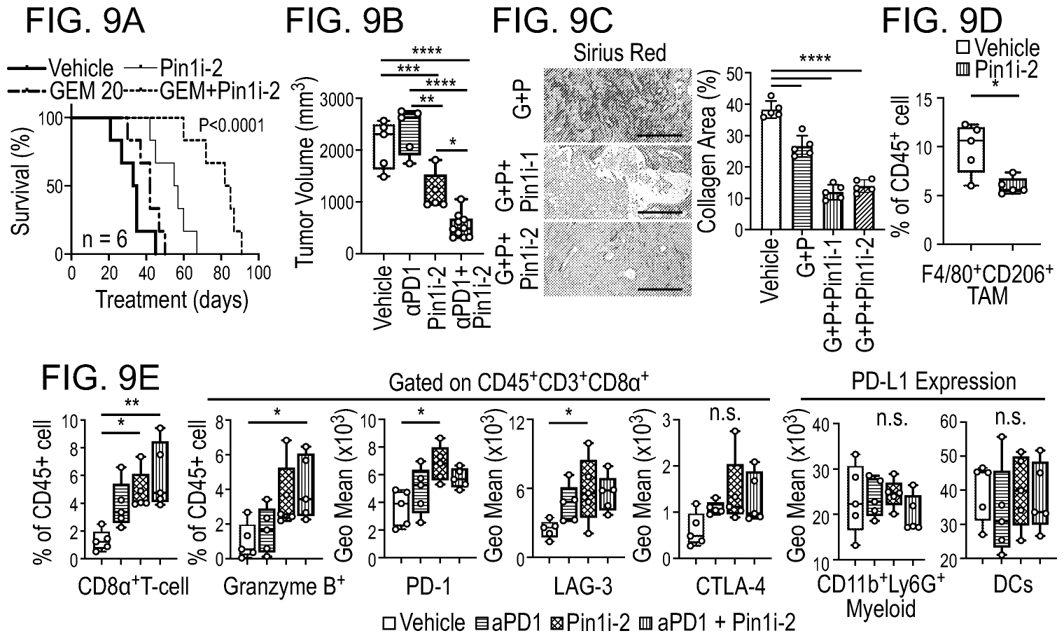

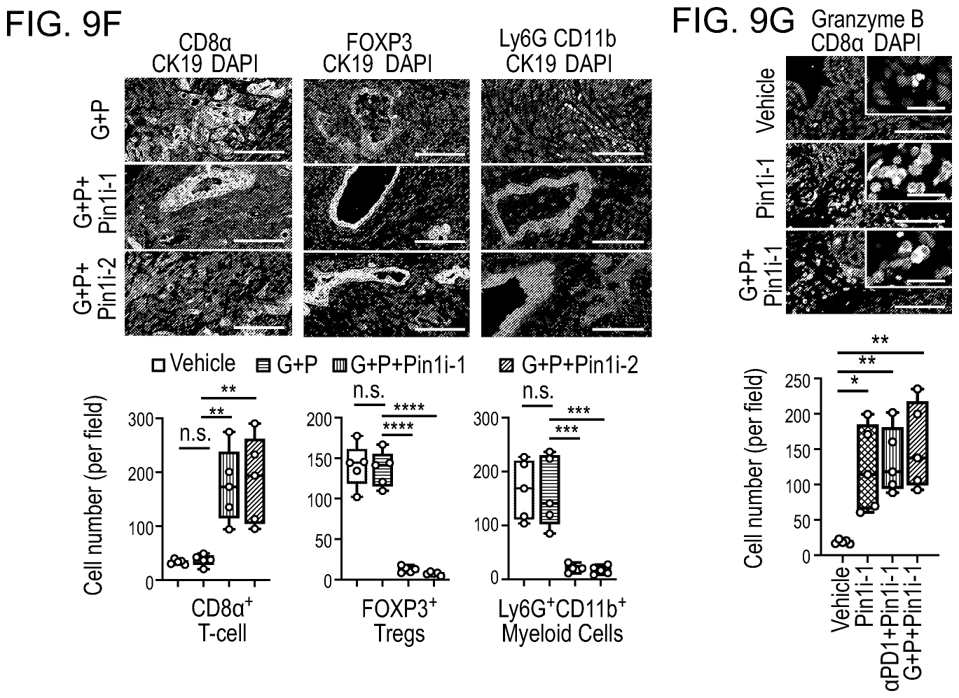

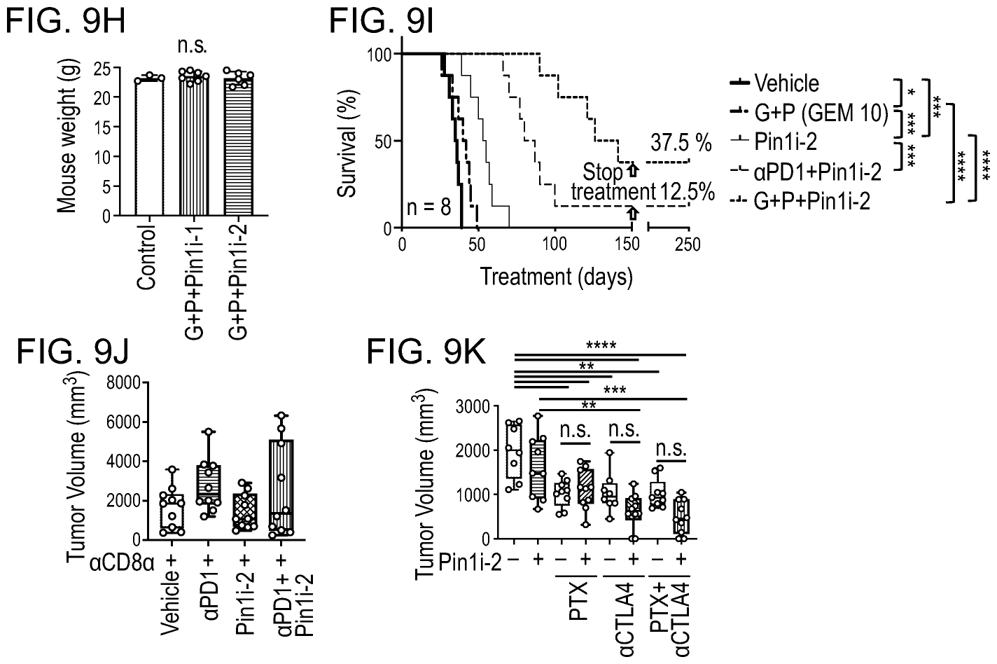

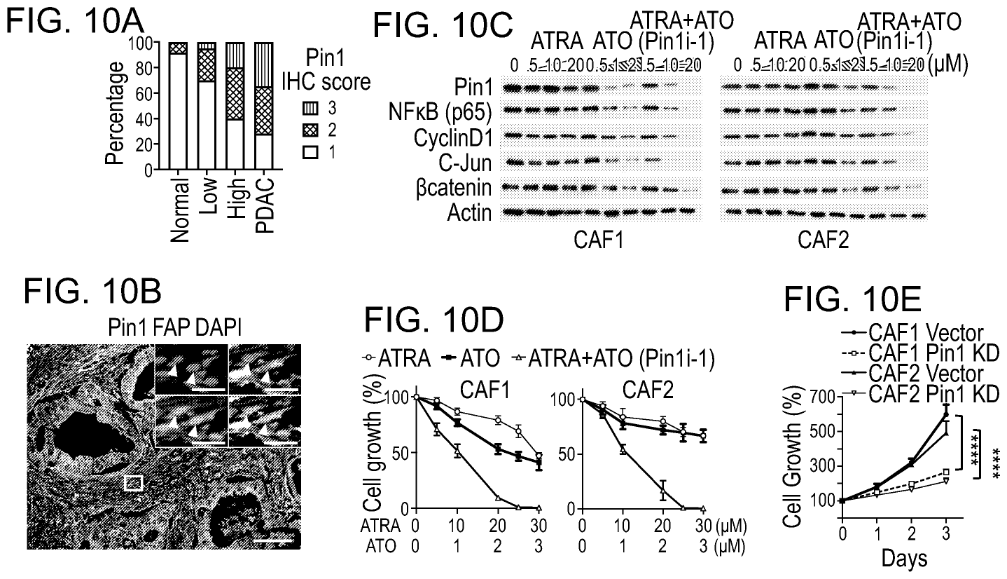

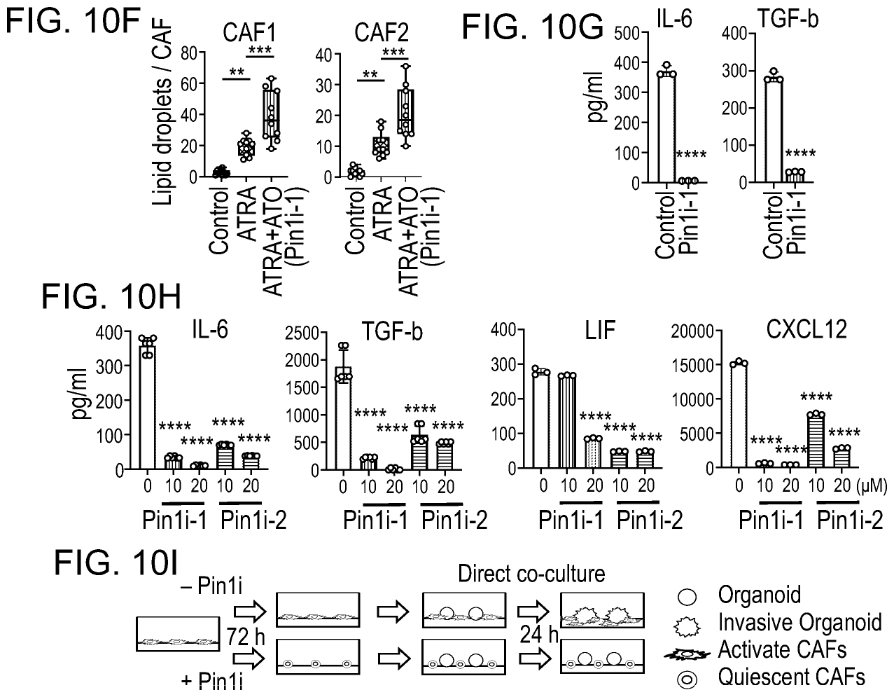

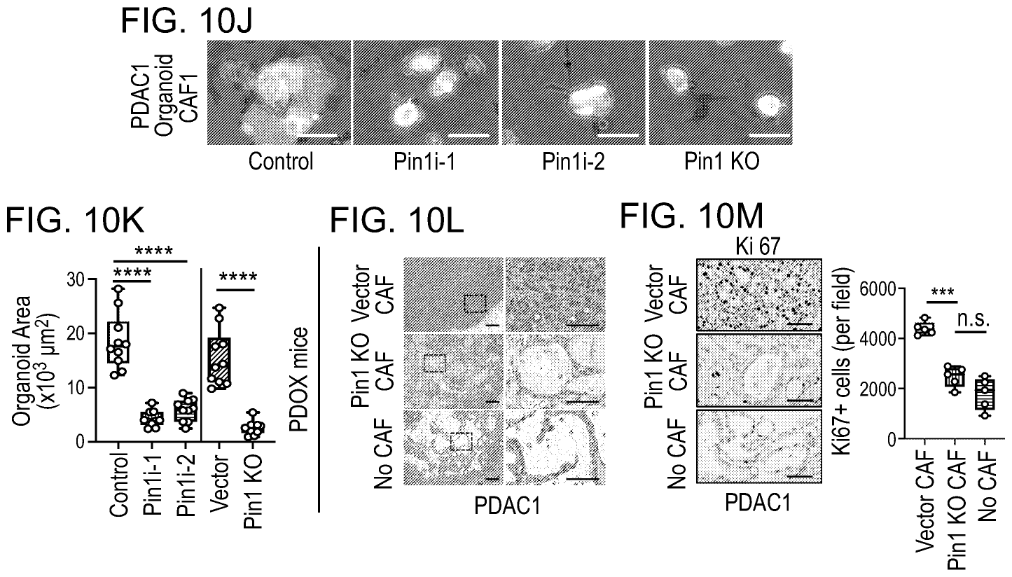

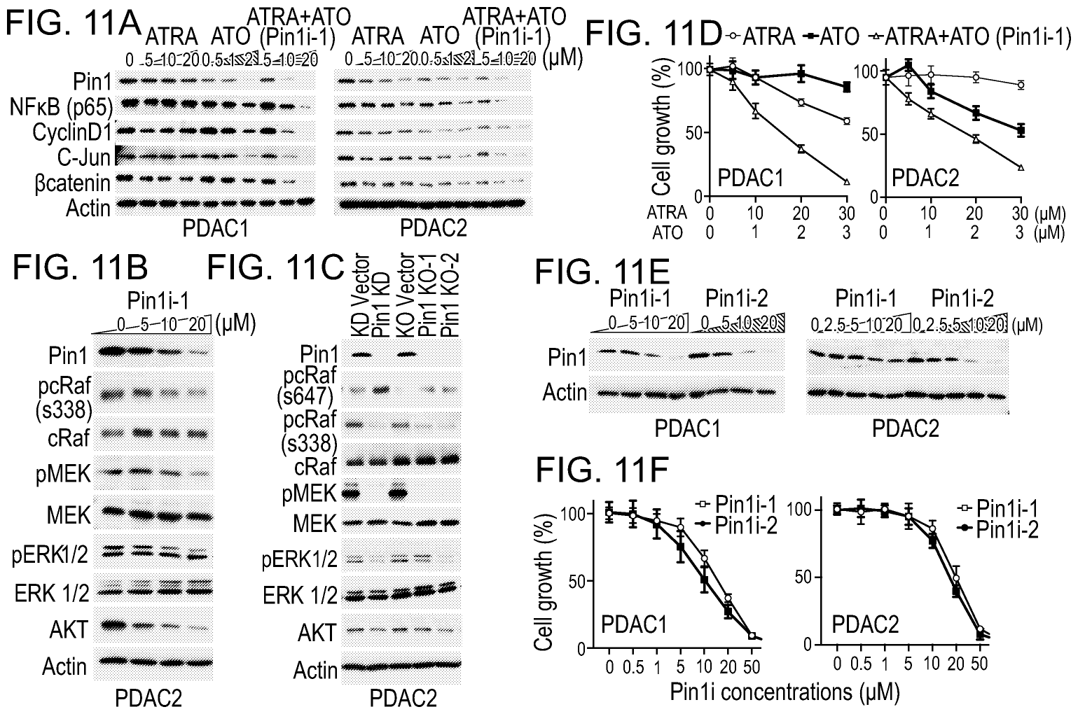

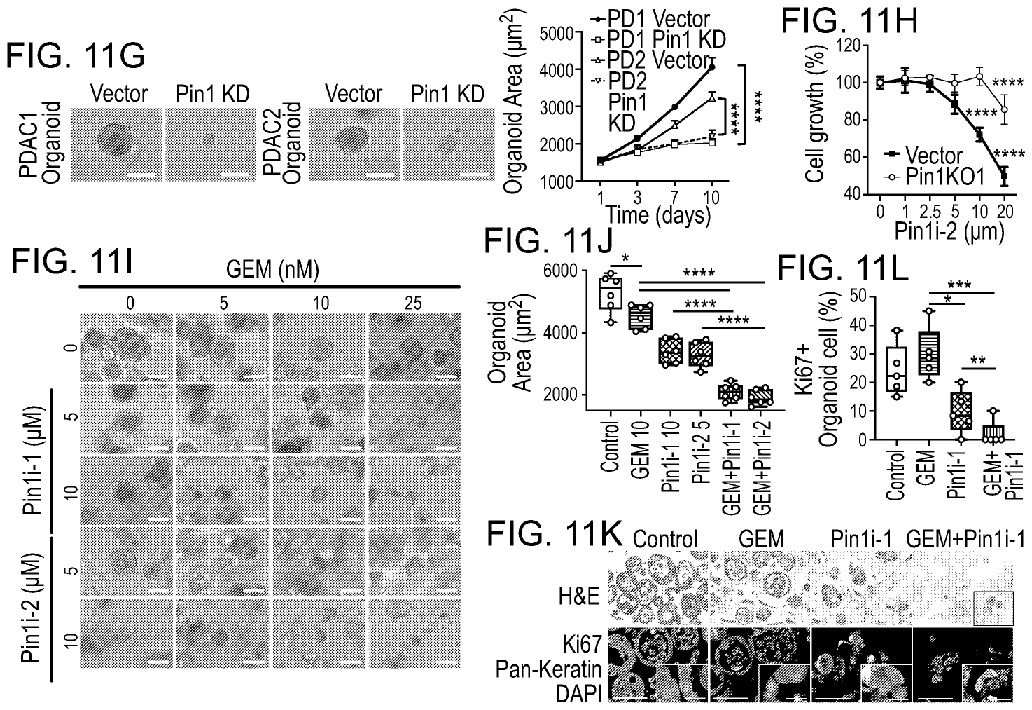

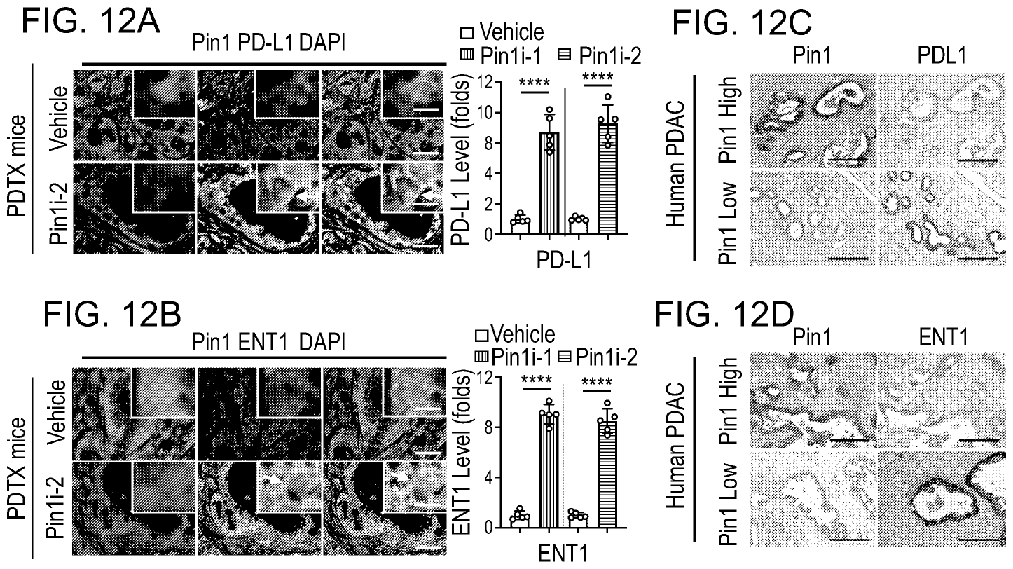

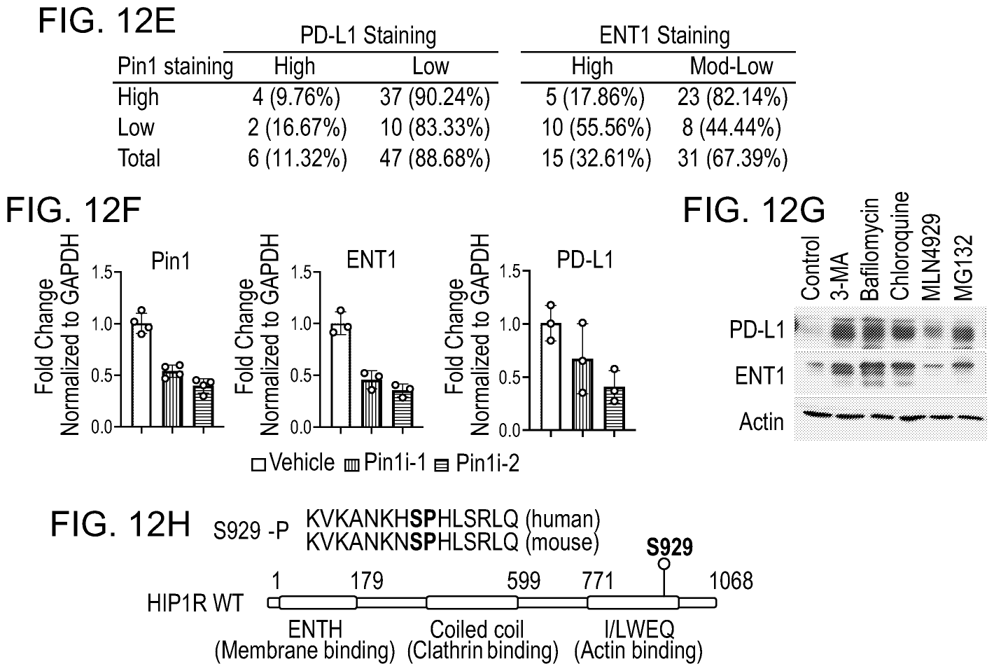

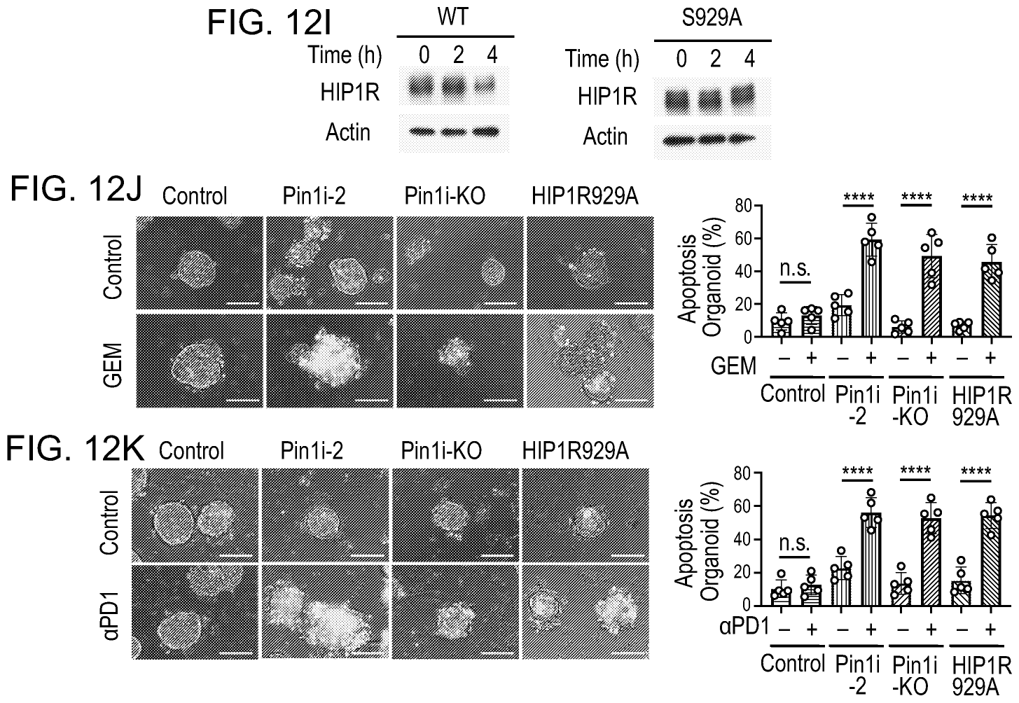

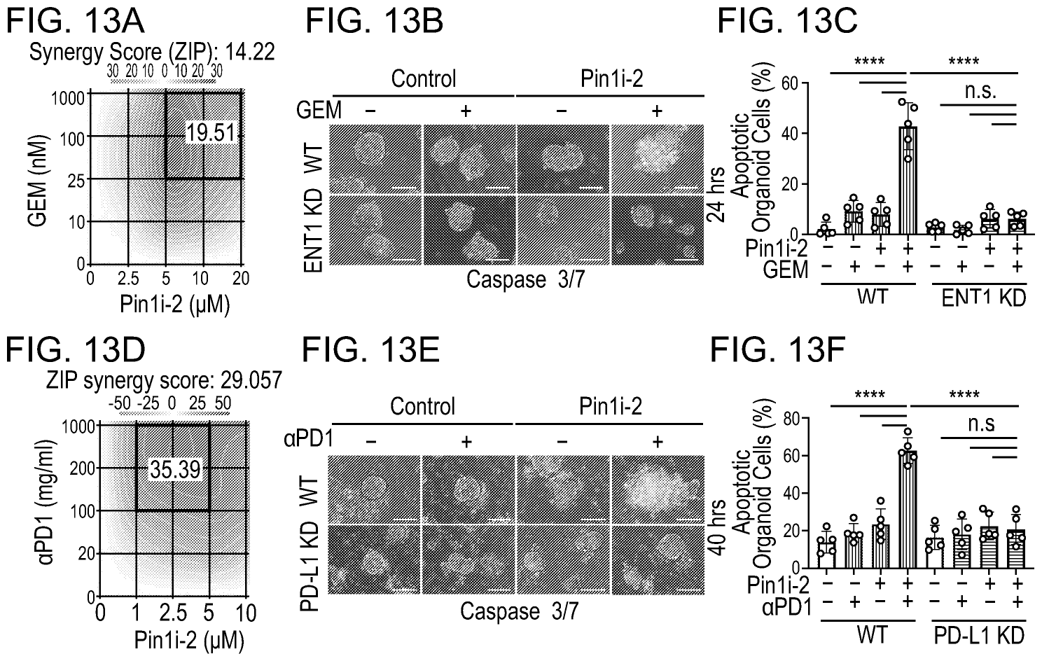

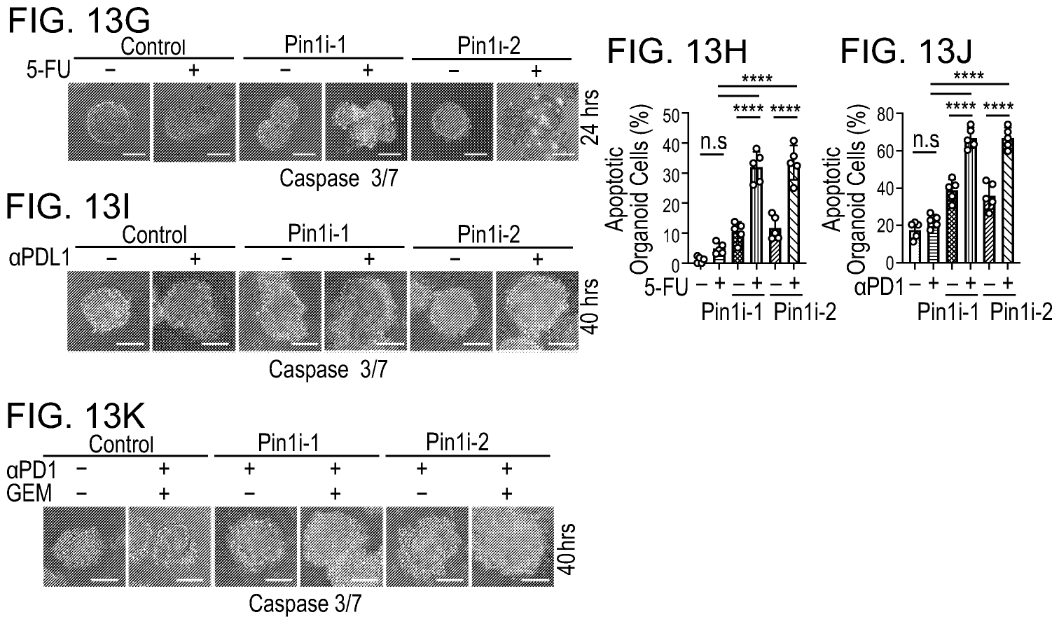

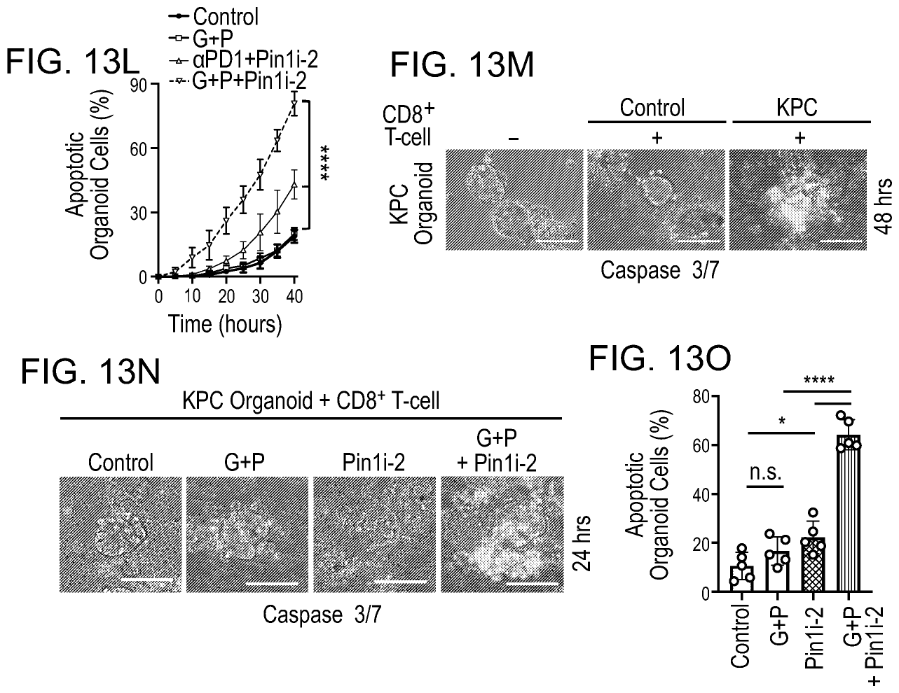

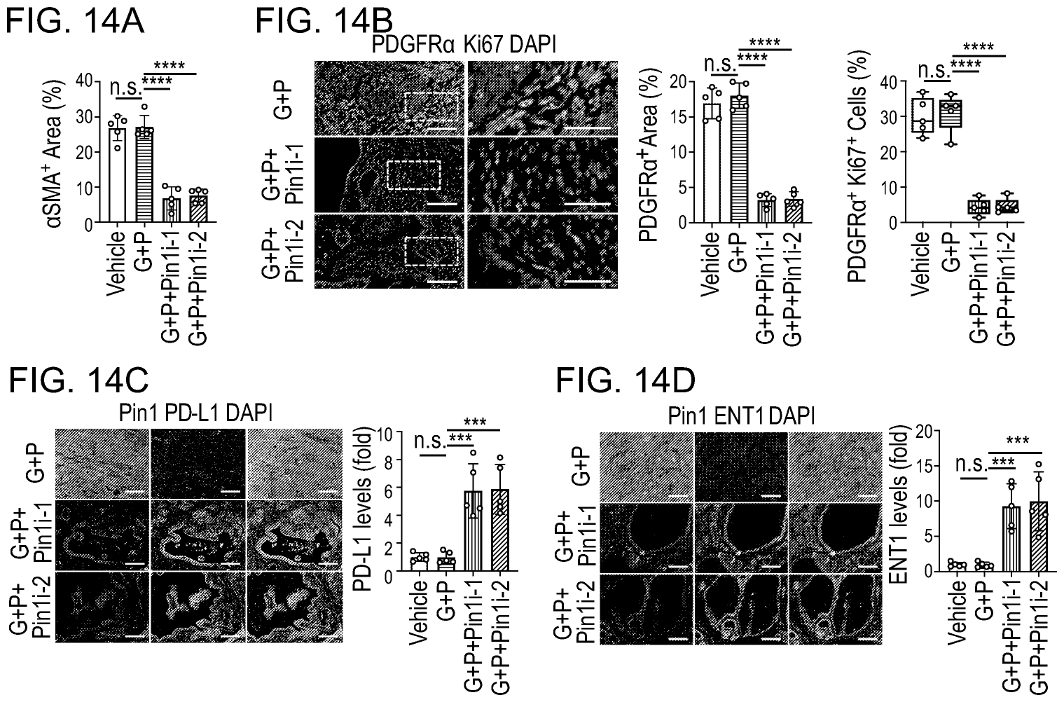

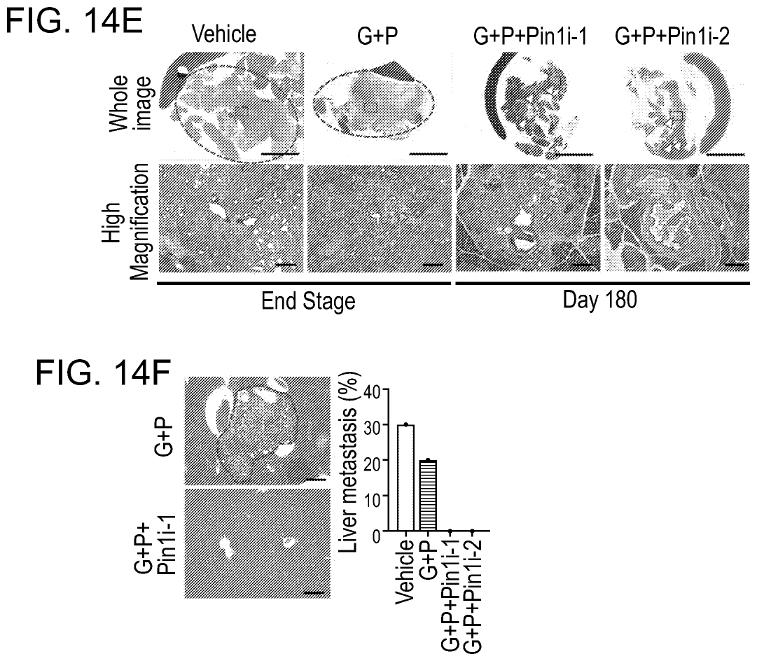

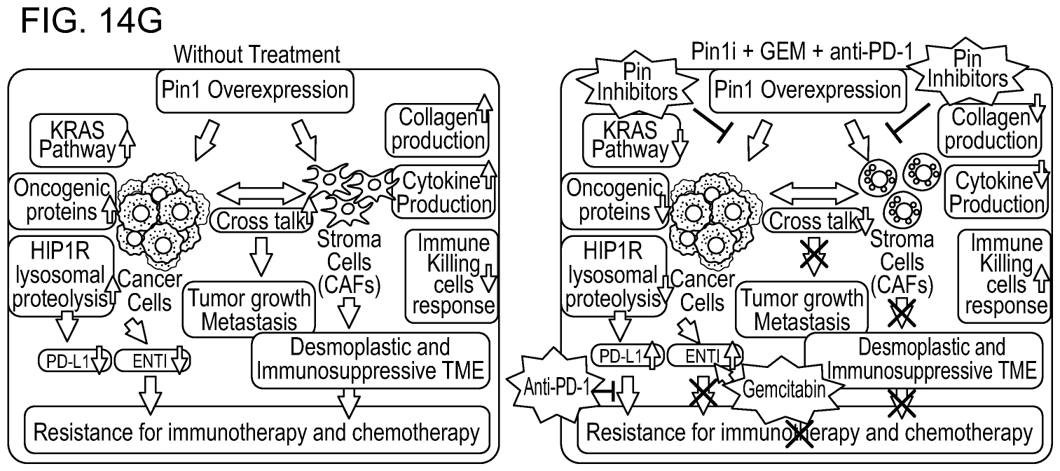

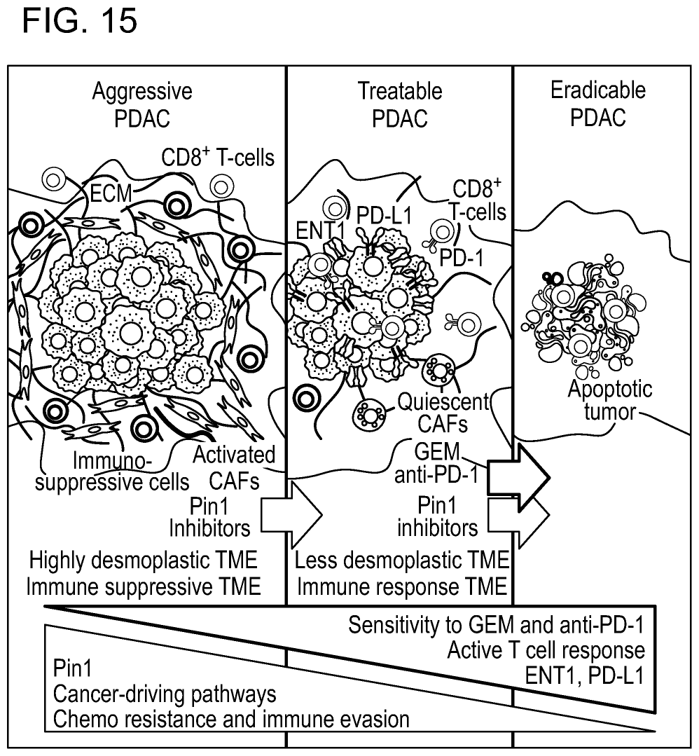

Subsequent experiments were shown with one organoid (PDAC2 organoid) with high expression of PD-L1 and ENT. FIG. 6C-FIG. 6E show that Pin1 inhibitors synergize with GEM to induce human PDAC organoid apoptosis in a time- and dose-dependent manner. Pin1i−1-pretreated PDAC organoids were treated with GEM, followed by examining PDAC organoid apoptosis for different times at constant concentrations (FIG. 6C, FIG. 6D; Pin1i−1 at 10 μM and GEM at 25 nM) or for 24 hrs at different concentrations and analyzing the synergy score of Pin1i and GEM using Synergy finder (FIG. 6E). FIG. 6F-FIG. 6H show that Pin1 inhibitors synergize with αPD1 to induce human PDAC organoid apoptosis. Pin1i−1-pretreated PDAC organoids were co-cultured with activated human PBMCs and treated with αPD1, followed by examining PDAC organoid apoptosis for different times at constant concentrations (FIG. 6F, FIG. 6G; Pin1i−1 at 10 μM and αPD1 at 200 g/mL) or for 40 hrs at different concentrations indicated and analyzing synergy score of Pin1i and αPD1 using Synergy finder (FIG. 6H). FIG. 6I-FIG. 6K show that Pin1 inhibitors synergize with immunochemotherapy (GEM+αPD1) to induce human PDAC organoid apoptosis.

Pin1i-pretreated PDAC organoids were co-cultured with activated human PBMCs, and then treated with GEM and/or 0PD1, followed by assaying PDAC organoid apoptosis for different times at constant concentrations (FIG. 6I, FIG. 6J; Pin1i−1 at 10 PM, GEM at 10 nM and αPD1 at 100 g/mL) or for 40 hrs at different concentrations indicated, and analyzing synergy score of Pin1i, GEM and/or αPD1 using Synergy finder (FIG. 6K). FIG. 6L-FIG. 6N show that the KPC tumor-bearing mouse derived CD8+ T-cells induces KPC mouse PDAC organoid apoptosis (FIG. 6L), and Pin1 inhibitors synergize with αPD1 to induce the KPC organoid apoptosis (FIG. 6M, FIG. 6N). KPC organoids derived from PDAC tumors in KPC mice were co-cultured with/without activated CD8+ T-cells derived from the same KPC tumor-bearing mouse or their tumor-free littermate that did not have all the three transgene (FIG. 6L), or the KPC organoids were pre-treated with control (DMSO) or Pin1i−1 at 10 at μM for 3 days, and co-cultured with the same KPC mouse tumor-bearing mouse derived activated CD8+ T-cells, and then treated with control (IgG) or αPD1 (FIG. 6M-FIG. 6N), followed by time-lapse live imaging to detect apoptosis of PDAC organoids using caspase 3/7 red fluorescent reagent for 48 hrs. (FIG. 6L) or 24 hrs (FIG. 6M, FIG. 6N). Scale bars 100 μm (FIG. 6C, FIG. 6F, FIG. 6I, FIG. 6M). Error bars represent mean±s.d.; *p<0.05, **p<0.001, ***p=0.0001, ****p<0.0001; n.s., not significant; by one-way ANOVA for multiple comparisons. See also FIG. 13A-FIG. 13O.

FIG. 7A is a set of IF images and a graph for GEM plus anti-PD-1, GEM plus anti-PD-1 plus Pin1i−1, and GEM plus anti-PD-1 plus Pin1i−2 showing CAF activation for αSMA and Ki67. FIG. 7B is a Sirius Red image and a graph of collagen area % for GEM plus anti-PD-1, GEM plus anti-PD-1 plus Pin1i−1, and GEM plus anti-PD-1 plus Pin1i−2. FIG. 7C is a set of IF images and a graph for GEM plus anti-PD-1, GEM plus anti-PD-1 plus Pin1i−1, and GEM plus anti-PD-1 plus Pin1i−2 showing CAF activation for Pan-Keratin and Ki67. FIG. 7D is a set of IF images and a graph for GEM plus anti-PD-1, GEM plus anti-PD-1 plus Pin1i−1, and GEM plus anti-PD-1 plus Pin1i−2 showing CAF activation for tumor-infiltrating immune cell populations, CD8α+ T-cells, FOXP3+ Tregs, and Ly6G+CD11b+ Myeloid cells. FIG. 7E is a set of IF images and a graph for GEM plus anti-PD-1, GEM plus anti-PD-1 plus Pin1i−1, and GEM plus anti-PD-1 plus Pin1i−2 showing CAF activation for CD8α+Granzyme B+ CTLs. FIG. 7F is a graph of survival % versus treatment days for GEM plus anti-PD-1, GEM plus anti-PD-1 plus Pin1i−1, and GEM plus anti-PD-1 plus Pin1i−2. FIG. 7G is a set of images of PDAC tumors in KPD transgenic mice for GEM plus anti-PD-1 end-stage, and GEM plus anti-PD-1 plus Pin1i−1, and GEM plus anti-PD-1 plus Pin1i−2 at day 180 together with H&E staining for macroscopic and microscopic tumors after autopsy.

FIG. 7A-FIG. 7G show that targeting Pin1 renders primary PDAC tumors eradicable by immunochemotherapy in genetically engineered KPC transgenic mice. FIG. 7A-FIG. 7E show that Pin1 inhibitors disrupt the desmoplastic and immunosuppressive TME in KPC transgenic mice. Overt tumor-bearing KPC mice were treated with vehicle or low dose GEM at 10 mg/kg (i.p., weekly)+αPD1 (G+P) with/without Pin1i−1 or Pin1i−2 for a month, followed by assaying CAF activation using IF for αSMA and CAF proliferation using double IF for αSMA and Ki67 (FIG. 7A), cancer cell proliferation using double IF for Pan-Keratin and Ki67 (FIG. 7C), tumor fibrosis using Sirius red staining (FIG. 7B) and tumor-infiltrating immune cell populations, CD8α+ T-cells, FOXP3+ Tregs, and Ly6G+CD11b+ Myeloid cells (FIG. 7D) and CD8α+Granzyme B+ CTLs (FIG. 7E) using IF (n=5). FIG. 7F and FIG. 7G show that Pin1 inhibitors render PDAC tumors eradicable by immunochemotherapy in KPC transgenic mice. Overt tumor-bearing KPC mice were treated with G+P with or without Pin1i−1 or Pin1i−2 for up to 180 days, followed by monitoring overall survival (n=10) (FIG. 7F), as well as detecting macroscopic and microscopic tumors with H&E staining (FIG. 7G) after autopsy. Scale bars, 100 and 25 μm (inset) (FIG. 7A, FIG. 7C, FIG. 7E), 500 μm (FIG. 7B), 100 μm (FIG. 7D), and 200 μm (FIG. 7G). Error bars represent mean±s.d.; *p<0.05, **p<0.001, ***p=0.0001, ****p<0.0001; n.s., not significant; by log-rank test (FIG. 7F) one-way ANOVA for multiple comparisons (FIG. 7A-FIG. 7E). See also FIG. 14A-FIG. 14G.

FIG. 8A is a flow cartoon showing the experimental setup and treatment schedule for PDTX, PDOX and GDA mice treated with Pin1 inhibitors, GEM and or anti-PD-1. FIG. 8B is a representative ultrasound image of the initial tumor on day −3. FIG. 8C is a set of tumor images and a graph of tumor volume for PDTX tumors upon treatment with Pin1i−1. FIG. 8D is a set of Sirius Red and H&E staining images showing collagen deposition and cancer cell differentiation together with a graph of collagen area % for Pin1i−1. FIG. 8E is a graph of cancer cell proliferation using Ki67 cancer cells. FIG. 8F is an IHC image and graph showing tumor-associated CAF activation and proliferation for αSMA and Ki67. FIG. 8G is an IHC image and graph showing tumor-associated CAF activation and proliferation for PDGFRα and Ki67. FIG. 8H is an IHC image and graph showing tumor-associated CAF activation and proliferation for E-cadherin and vimentin. FIG. 8I is a set of images for Pin1i−1 and Pin1i−2 in overt tumor-bearing GDA mice using H&E staining. FIG. 8J is a set of images and two graphs showing tumor-associated CAF proliferation for PDGFRα and Ki67. FIG. 8K is a graph of tumor volume in PDOX mice for Pin1i−1 (Day 0) and GEM and Pin1i−1 (Day −3) and GEM. FIG. 8L is a graph of tumor volume in PDTX mice for Pin1i−1 (Day 0) and GEM and Pin1i−1 (Day −3) and GEM. FIG. 8M is a graph of % survival versus days for GEM, Pin1i−1, and GEM plus Pin1i−1. FIG. 8N is a set of images and a graph for liver metastasis % for GEM, Pin1i−1, and GEM plus Pin1i−1.

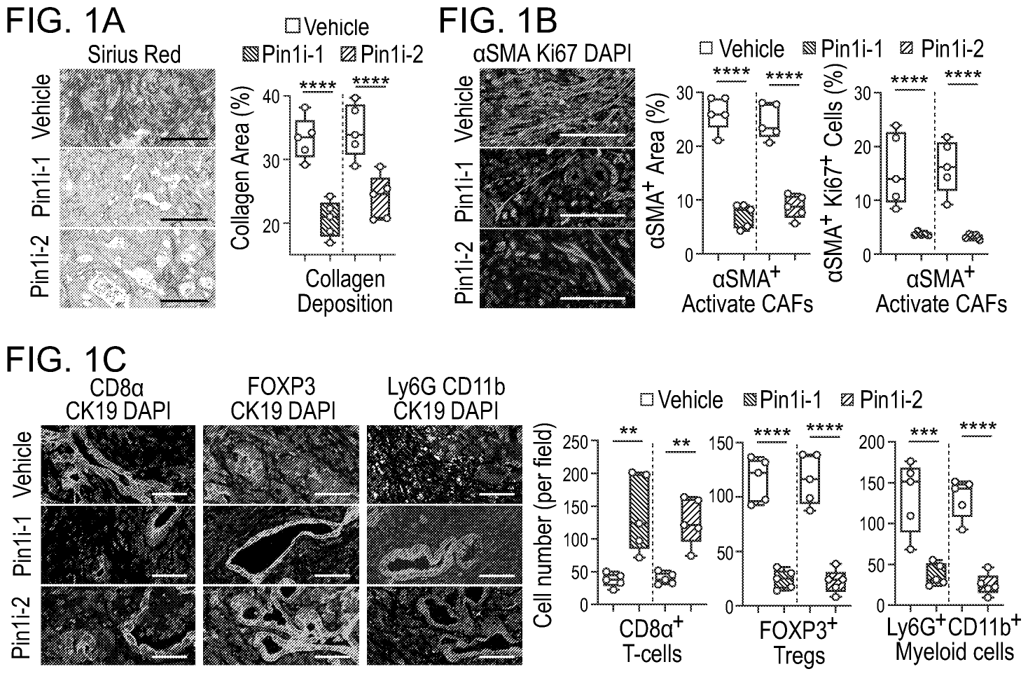

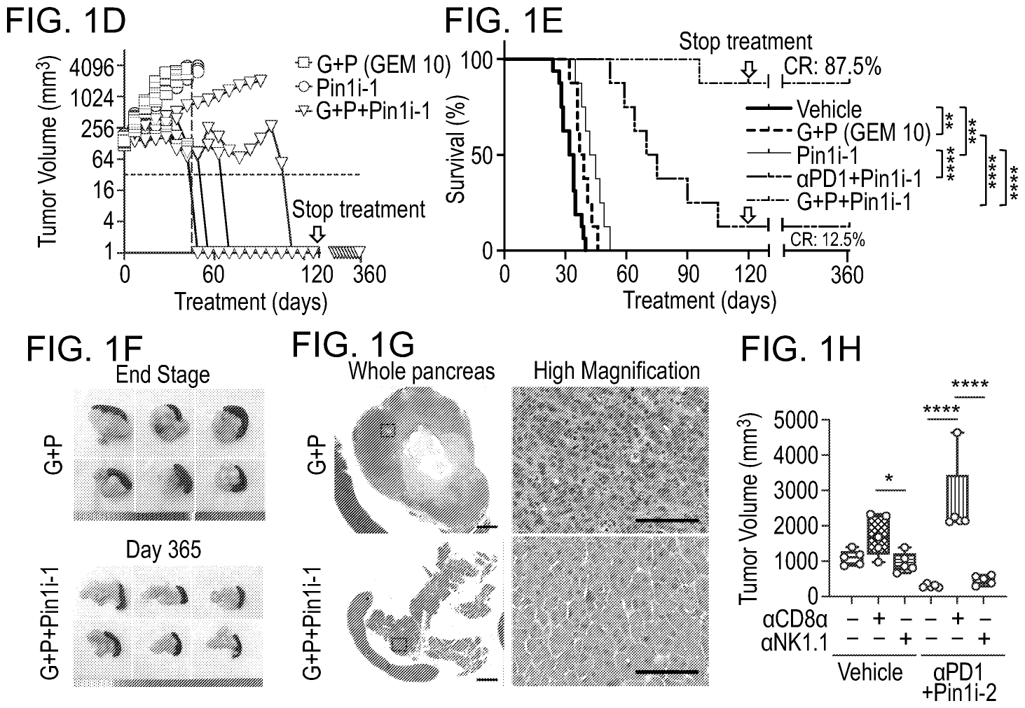

FIG. 8A-FIG. 8N show that targeting Pin1 disrupts the desmoplastic and/or immunosuppressive TME and inhibits tumor growth and progression in patient-derived tumor orthotopic xenograft (PDTX) and GDA mice. See also FIG. 1A-FIG. 1G. FIG. 8A-FIG. 8B show the experimental setup and treatment schedule of Pin1i, together with a representative ultrasound image. Tumor-bearing (>0.5 cm) PDTX, PDOX or GDA mice were treated with Pin1i, GEM and/or αPD1, starting Pin1i 3 days before others unless stated otherwise. Tumor sizes were detected by ultrasound or palpation with electronic caliper (FIG. 8A). A representative ultrasound image of initial tumor on Day −3 is shown in FIG. 8B. FIG. 8C-FIG. 8H show that Pin1 inhibitors suppress tumor growth and progression, and desmoplastic TME in PDTX mice. Overt tumor-bearing PDTX mice were treated with vehicle, Pin1i−1 or Pin1i−2 for 4 weeks, followed by assaying tumor growth and volume (FIG. 8C), collagen deposition and cancer cell differentiation using Sirius Red and H&E staining (FIG. 8D), cancer cell proliferation using Ki67 IHC (FIG. 8E), and tumor-associated CAF activation and proliferation using double IF for αSMA and Ki67 (FIG. 8F) or PDGFRα and Ki67 (FIG. 8G), and EMT using double IF for E-cadherin and vimentin (FIG. 8H) (n=5). White arrows indicate αSMA and Ki67 double positive CAFs (FIG. 8F), Ki67 and PDGFRα double positive CAFs (FIG. 8G), or vimentin positive cancer cell (FIG. 8H). FIG. 8I-FIG. 8J show that Pin1 inhibitors suppress cancer cell proliferation and tumor progression, and desmoplastic and immunosuppressive TME in GDA mice. Overt tumor-bearing GDA mice were treated with Pin1i−1 or Pin1i−2 for 4 weeks, followed by examining cancer cell differentiation using H&E staining (FIG. 8I), tumor-associated CAF proliferation using double IF for PDGFRα and Ki67 using IF (FIG. 8J) (n=5). FIG. 8K-FIG. 8N show that Pin1 inhibitors render PDAC sensitive to GEM in PDOX and PDTX mice. Patient-derived PDAC1 organoids and CAFs (FIG. 8K; PDOX mice, n=5) or patient-derived PDAC tumors (FIG. 8L-FIG. 8N; PDTX mice, n=6) were orthotopically transplanted into NSG mouse pancreas and when tumors reached 0.5 cm, mice were treated with vehicle, Pin1i and/or GEM (20 mg/kg), followed by measuring tumor volumes (FIG. 8K and FIG. 8L) and liver metastasis (FIG. 8N) after autopsy at 4 weeks after treatment, or monitoring overall survival by Kaplan-Meier survival analysis (FIG. 8M). Median survival; vehicle 36 days, GEM 42.5 days, Pin1i−1 43 days, and Pin1i−1+GEM 71 days (FIG. 8M). Scale bars; 500 and 100 μm (right panel) (FIG. 8D), 100 and 25 μm (inset) (FIG. 8F, FIG. 8G), 50 μm (FIG. 8H, FIG. 8J), and 100 μm (FIG. 8I), and 200 μm (FIG. 8N). Error bars represent mean±s.d.; *p<0.05, **p<0.001, ***p=0.0001, ****p<0.0001; n.s., not significant; by log-rank test (FIG. 8O) by unpaired two-sided Student's t test (FIG. 8C-FIG. 8H, FIG. 8J) or one-way ANOVA for multiple comparisons (FIG. 8K, FIG. 8L, FIG. 8N), or log-rank test (FIG. 8M).

FIG. 9A-FIG. 9K is a set of line plots, box plots, bar plots, and images showing that targeting Pin1 disrupts the desmoplastic and/or immunosuppressive TME and renders PDAC sensitive to GEM. FIG. 9A is a line plot that shows Pin1 inhibitors render PDAC sensitive to GEM in GDA mice. FIG. 9B is a box plot that shows tumor volume in mice after treatment. FIG. 9C is a set of Sirius Red images showing percentage of collagen area after GEM (10 mg/kg)+αPD1 (200 pg/mouse) (G+P) with and without Pin1i. FIG. 9D is a box plot showing immune profiling using flow cytometry. FIG. 9E is a set of box plots showing immune cell subsets using flow cytometry. FIG. 9F is a set of images and box plots showing tumor-infiltrating immune cell populations. FIG. 9G is a set of images and a box plot showing tumor-infiltrating immune cell populations. FIG. 9H is a bar plot showing mouse body weight. FIG. 9I is a line plot showing mouse survival. FIG. 9J shows that αPD1 renders GDA tumors hyperprogressive in the absence of CD8+ T-cells. FIG. 9K shows that Pin1 inhibition does not significantly potentiate Paclitaxel or anti-CTLA4 therapy in GDA mice.

FIG. 9A-FIG. 9K show that targeting Pin1 disrupts the desmoplastic and/or immunosuppressive TME and renders PDAC sensitive to GEM in PDTX, PDOX and GDA mice. See also FIG. 1A-FIG. 1G. FIG. 9A shows that Pin1 inhibitors render PDAC sensitive to GEM in GDA mice. Overt-tumor-bearing GDA mice were treated with vehicle, Pin1i and/or GEM (20 mg/kg) (n=6), followed by monitoring overall survival by Kaplan-Meier survival analysis. Median survival; vehicle 34 days, GEM 42 days, Pin1i−2 56 days, GEM+Pin1i−2 83.5 days. FIG. 9B-FIG. 9H show that Pin1 inhibitors render PDAC sensitive to GEM and αPD1 by disrupting the desmoplastic and immunosuppressive TME in GDA mice. Overt-tumor-bearing GDA mice were treated with vehicle, Pin1i and/or αPD1 (200 pg/mouse) or GEM (10 mg/kg)+αPD1 (200 pg/mouse) (G+P), followed by measuring tumor volumes (FIG. 9B), assaying collagen deposition using Sirius Red staining (FIG. 9C), immune profiling using flow cytometry (FIG. 9D, FIG. 9E), and tumor-infiltrating immune cell populations CD8α+ T-cells, FOXP3+ Tregs, Ly6G+CD11b+ Myeloid cells (FIG. 9F), and CD8α+Granzyme B+ CTLs (FIG. 9G) using IF after 4 weeks treatment (n=5), or measuring body weight after 120 days treatment (Control/No transplantation: n=3, G+P+Pin1i−1: n=7, G+P+Pin1i−2: n=6) (FIG. 9H) Pin1 inhibitors render PDAC eradicable by immunochemotherapy in GDA mice. Overt tumor-bearing GDA mice were treated with vehicle, Pin1i−2, low dose GEM at 10 mg/kg (i.p., weekly)+αPD1 at 200 pg/mouse (G+P), Pin1i−2+0PD1, or Pin1i−2+G+P for up to 150 days and followed by monitoring overall survival by Kaplan-Meier survival analysis up to 250 days (n=8). Median survival; vehicle 36.5 days, G+P 41 days, Pin1i−2 54 days, αPD1+Pin1i−2 83.5 days, G+P+Pin1i−2 133.5 days (FIG. 9I). FIG. 9J shows that αPD1 renders GDA tumors hyperprogressive in the absence of CD8+ T-cells. Overt tumor-bearing GDA mice were treated with αCD8α and vehicle, αPD1, Pin1i−2, or αPD1+Pin1i−2 for 9 days, followed by examining tumor volume. FIG. 9K shows that Pin1 inhibition does not significantly potentiate Paclitaxel or anti-CTLA4 therapy in GDA mice. Overt tumor-bearing GDA mice were treated with vehicle, Pin1i−2, Paclitaxel (PTX), anti-CTLA 4 (αCTLA4), or their combination for 4 weeks, followed by assaying tumor volume. Scale bars, 200 μm (FIG. 9C), 100 m (FIG. 9F), and 100 and 25 μm (inset) (FIG. 9G). Error bars represent mean±s.d.; *p<0.05, **p<0.001, ***p=0.0001, ****p<0.0001; n.s., not significant; by log-rank test (FIG. 9A, FIG. 9I), unpaired two-sided Student's t test (FIG. 9D), or one-way ANOVA for multiple comparisons (FIG. 9B-FIG. 9C, FIG. 9E-FIG. 9H, FIG. 9J-FIG. 9K).

FIG. 10A-FIG. 10M is a set of bar plots, immunoblots, images, and box plots showing that Pin1 is overexpressed in CAFs and promotes oncogenic signaling pathways. FIG. 10A is a bar plot that shows Pin1 overexpression in human PDAC tissues. FIG. 10B is an image that shows Pin1 overexpression in CAFs by co-IF for Pin1 (red) and FAP (green). FIG. 10C is a set of immunoblots that show ATRA and ATO synergistically reduce Pin1 and its substrate oncoprotein. FIG. 10D is a set of line plots that show ATRA and ATO synergistically reduce cell growth. FIG. 10E is a line plot that shows the effect of Pin1 KD on cell growth in CAF1 or CAF2 cells. FIG. 10F is two box plots that show ATRA and ATO synergistically affect lipid droplet in CAF1 and CAF2 cells. FIG. 10G is two bar plots that show IL-6 and TGF-β protein expression. FIG. 10H is four bar plots that show Pin1 inhibitors reduce cytokine production CAF cells. FIG. 10I is a graphical representation of Pin1-inhibited or CRISPR KO CAFs fail to promote PDAC growth and invasion in human 3D PDAC organoid direct co-cultures. FIG. 10J is a set of images that show Pin1 inhibitors effect on PDAC1 organoids. FIG. 10K is a box plot that quantifies organoid area with Pin1 inhibitors or Pin1 KD. FIG. 10L is a set of images of H&E histology staining and a box plot that show that Pin1-KO CAFs fail to promote PDAC tumor progression and proliferation in PDOX mice. FIG. 10M is a set of immunohistochemistry images of Ki67 cells analyzing cell proliferation with and without Pin1 KO.

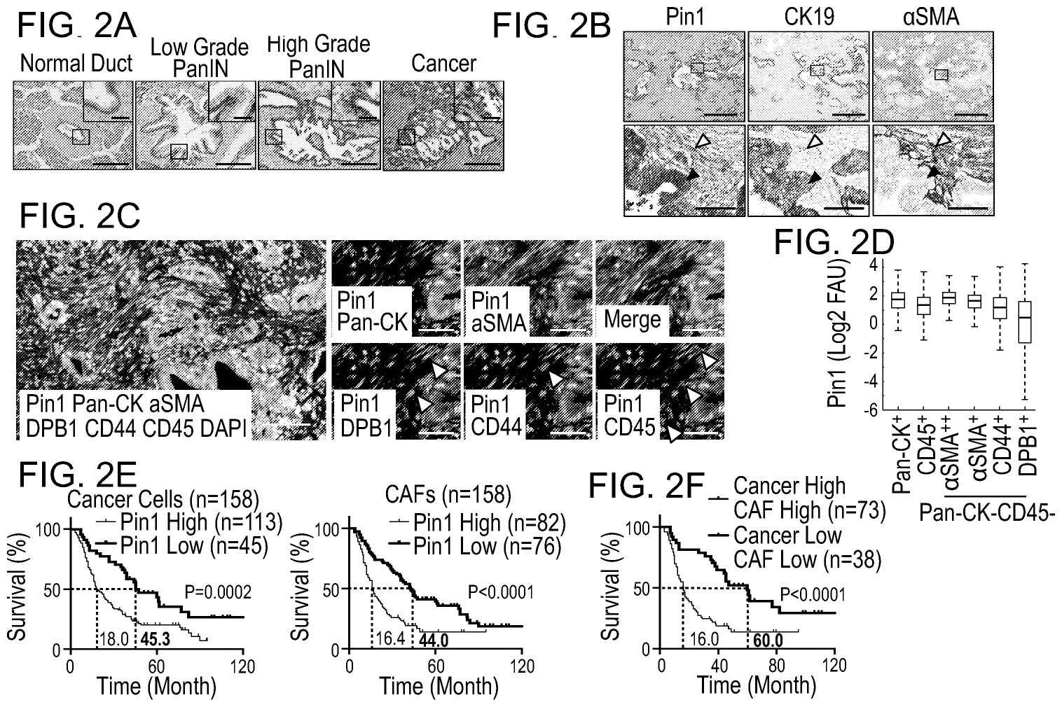

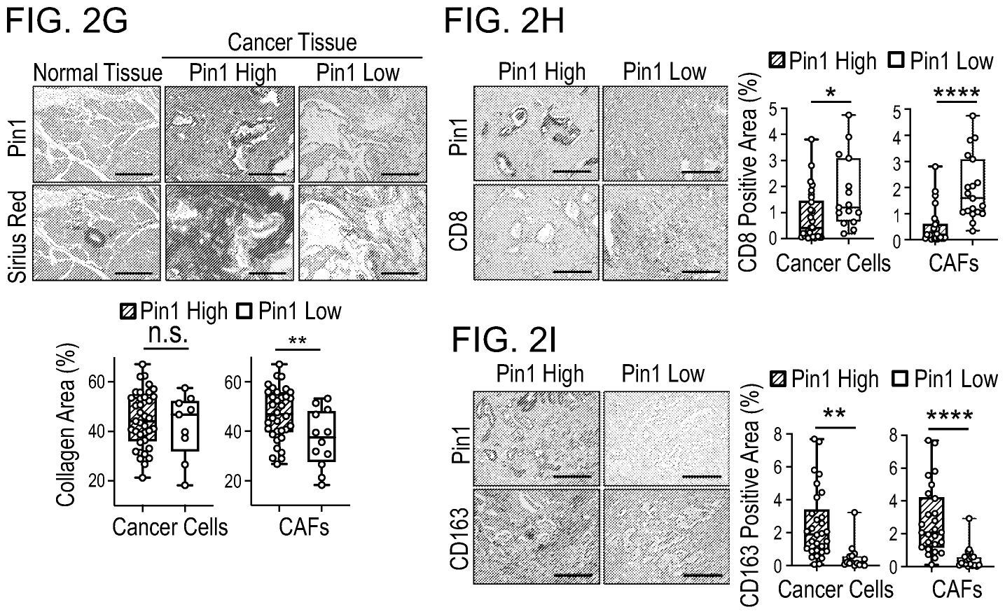

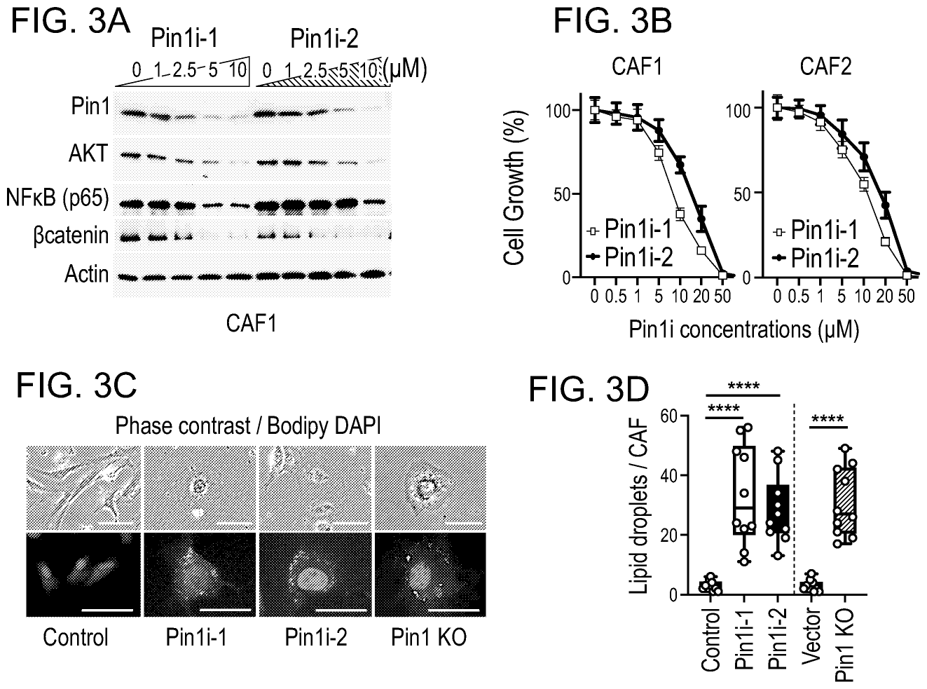

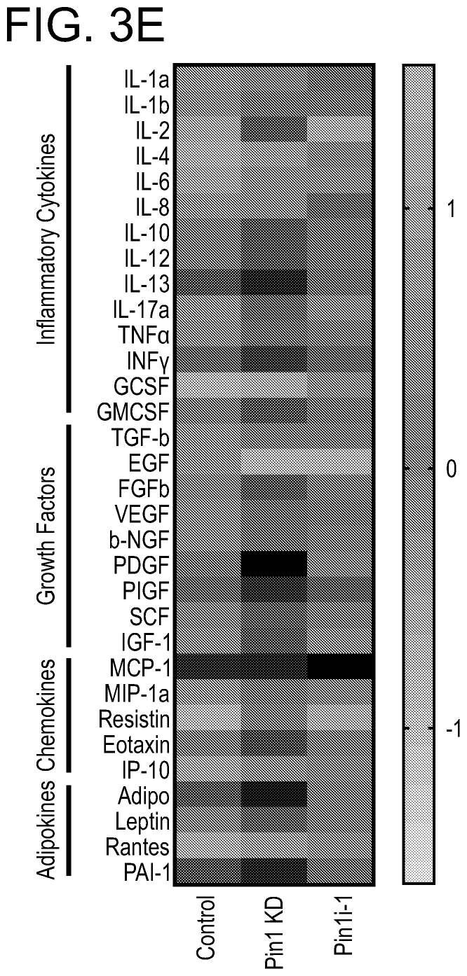

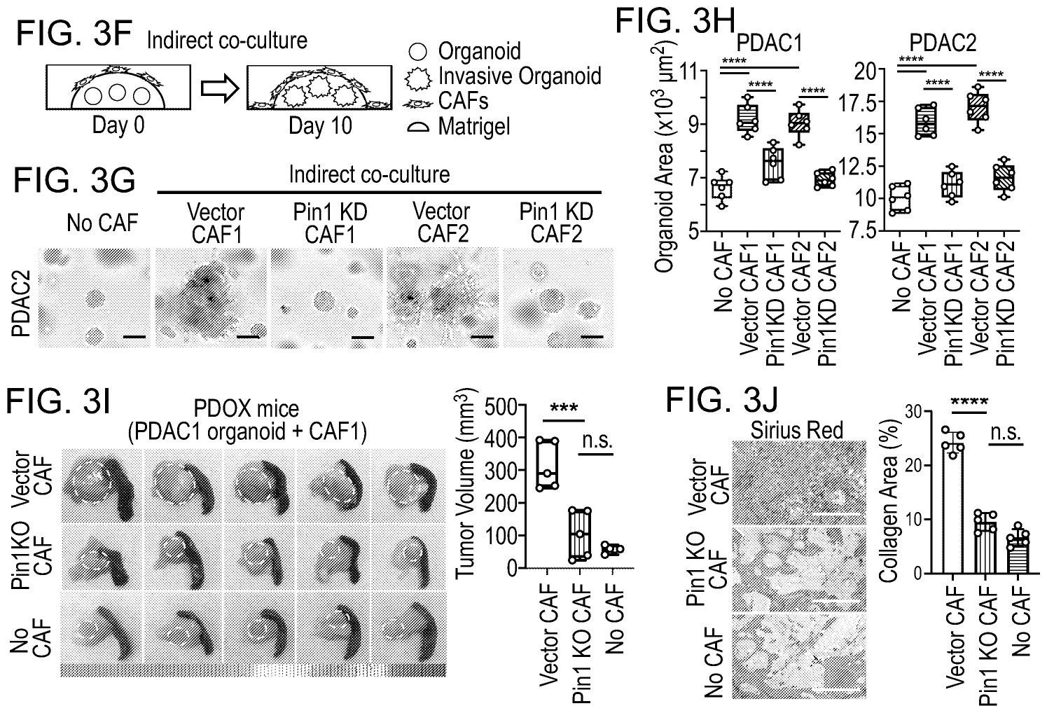

FIG. 10A-FIG. 10M show that Pin1 is overexpressed in CAFs and promotes oncogenic signaling pathways, CAF activation and crosstalk with cancer cells to enhance tumor growth and malignancy in human organoids and PDOXs. See also FIG. 2A-2I and FIG. 3A-FIG. 3J. FIG. 10A-FIG. 10B show that Pin1 is overexpressed in cancer cells and CAFs and correlated with PDAC progression. FIG. 10A shows Pin1 overexpression in human PDAC tissues, as compared with normal controls, the relationship of Pin1 overexpression at different stages of PDAC cancer progression, followed by determining IHC scores in different stages (FIG. 10A), and FIG. 10B shows Pin1 overexpression in CAFs by co-IF for Pin and FAP (FIG. 10B). Normal-Adjacent normal (Normal, n=12), Low grade PanIN (Low, n=20), High grade PanIN (High, n=35), PDAC (n=167) (FIG. 10A). FIG. 10C-FIG. 10F show that ATRA and ATO synergistically reduce Pin1 and its substrate oncoproteins, suppress cell growth and induce quiescent phenotype in CAFs, like Pin1 KD. CAF1 and CAF2 cells derived from two different human PDAC tissues were treated with ATRA, ATO, or their combination (Pin1i−1) for 72 hrs (FIG. 10C, FIG. 10D, FIG. 10F) or genetically knocked down of Pin1 (FIG. 10E), followed by examining Pin1 and its substrate oncoproteins using IB (FIG. 10C), cell growth using cell proliferation assay (FIG. 10D, FIG. 10E), quiescent phenotype as measured by lipid droplets/CAF cell using BODIPY staining (FIG. 10F). ATRA and ATO were used in a 10:1 ratio as Pin1i−1 and only ATRA concentrations were shown. CAFs were treated with control (DMSO), ATRA at 10 μM or Pin1i−1 at 10 μM for 72 hrs (FIG. 10F). FIG. 10G and FIG. 10H show that Pin1 inhibitors reduce cytokine production in primary human PDAC cells (FIG. 10G) and CAFs (FIG. 10H). Primary human PDAC2 or CAF1 cells were treated with Pin1i−1 or Pin1i−2 for 72 hrs, before assaying IL-6, TGFb, LIF and CXCL12 using ELISA. FIG. 10I-FIG. 10K show that Pin1-inhibited or CRISPR KO CAFs fail to promote PDAC growth and invasion in human 3D PDAC organoid direct co-cultures. Control (DMSO) or Pin1i-treated CAF (Pin1il 10 μM or Pin1i−2 5 μM for 72 hrs), or Pin KO or CRISPR control CAFs, labeled in red using cell tracker red, were cocultured with established human PDAC organoids, labeled by GFP (FIG. 10I), followed by live-cell time-lapse movies to visualize their interactions and analyze organoid growth and invasion (FIG. 10J, FIG. 10K). FIG. 10 L-FIG. 10M show that Pin1-KO CAFs fail to promote PDAC tumor progression and proliferation in PDOX mice. Human PDAC1 organoids were orthotopically co-transplanted with or without Pin1 KO or CRISPER control CAFs into NSG mouse pancreas for 5 weeks, followed by examining histology using H&E staining (FIG. 10L) and cell proliferation using Ki67 IHC (FIG. 10M) derived from PDAC1 organoids (n=5). Scale bars, 100 and 20 μm (inset) (FIG. 10B), 100 μm (FIG. 10J), 100 and 50 μm (right panels) (FIG. 10L), and 50 μm (FIG. 10M). Error bars represent mean±s.d.; **p<0.05, **p<0.001, ***p=0.0001, ****p<0.0001; n.s., not significant; by unpaired two-sided Student's t test FIG. 10 (E, FIG. 10G) or one-way ANOVA for multiple comparisons (FIG. 10F, FIG. 10H. FIG. 10K, FIG. 10M).

FIG. 11A-FIG. 11L is a set of immunoblots, images, line plots, and box plots showing that Pin1 promotes oncogenic signaling pathways and growth of PDAC cells. FIG. 11A is a set of immunoblots that show ATRA and ATO cooperatively reduce Pin1 and its substrate oncoproteins in PDAC cells. FIG. 11B is a set of immunoblots that show the effect of Pin1i−1 KD in PDAC cells. FIG. 11C is a set of immunoblots that show Pin1 KD or Pin1 KO effects in PDAC cells.

FIG. 11D is a set of line plots that show ATRA, ATO, Pin1i−1 (ATRA+ATO) and Pin1i−2 (sulfopin) dose-dependently suppress cell growth. FIG. 11E is a set of immunoblots that show 1 and Pin1i−2 dose-dependently ablate Pin1. FIG. 11F is a set of line plots that show Pin1i−1 and Pin1i−2 dose-dependently suppress cell growth. FIG. 11G is a set of images and a line plot that shows Pin1 KD inhibits human PDAC organoid growth. FIG. 11H is a line plot that shows Pin1i−2 fails to suppress Pin1 KO cell growth. FIG. 11I is a set of images that shows Pin1 inhibitors enhance the ability of GEM to inhibit human PDAC organoid growth. FIG. 11J is a box plot that quantifies the organoid area in FIG. 13I. FIG. 13K is a set of images that show Pin1 inhibitors enhance GEM inhibition by H&E staining, and organoid proliferation using double IF for Pan-Keratin and Ki67 IF. FIG. 11L is a box plot showing that Pin1 inhibitors with GEM effect the percentage of Ki67+ organoid cells.

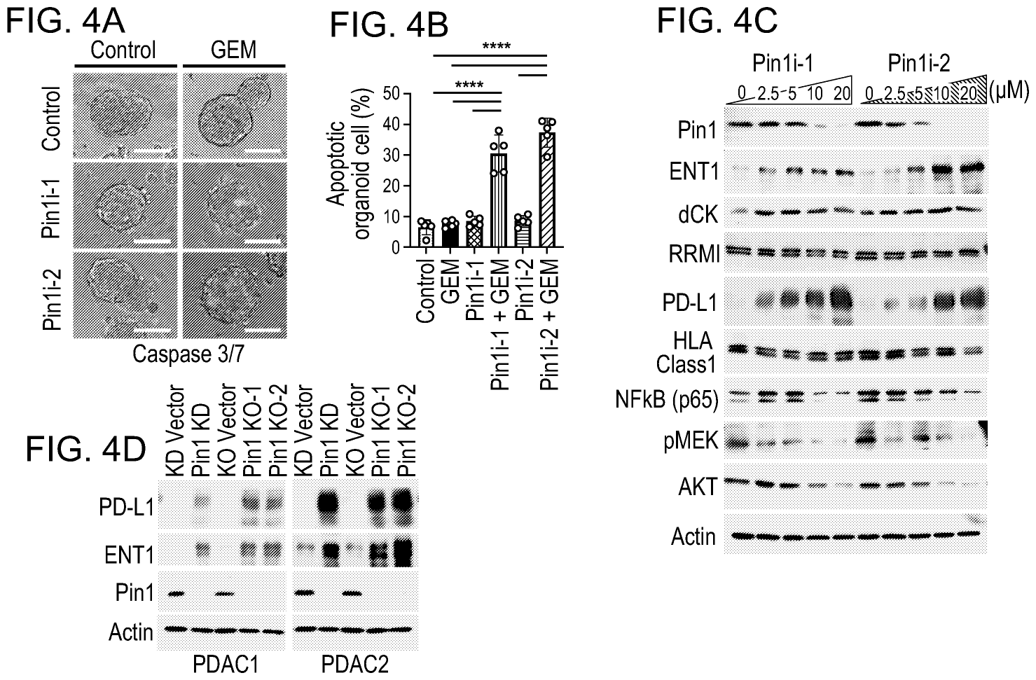

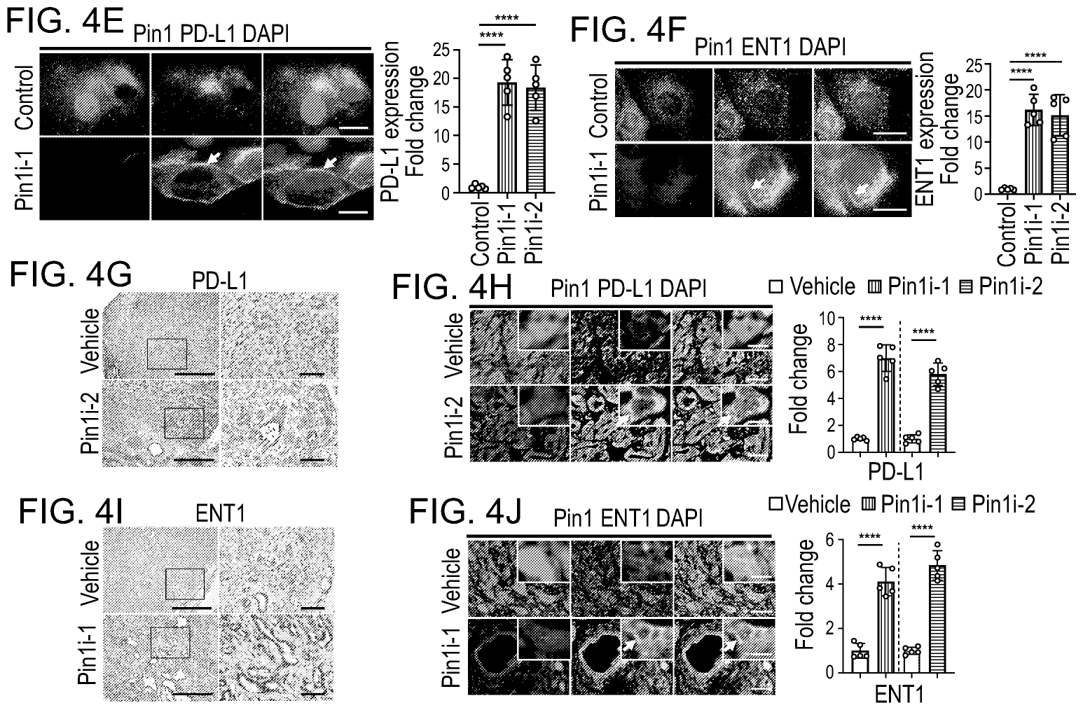

FIG. 11A-FIG. 11L show that Pin1 promotes oncogenic signaling pathways and growth of PDAC cells. See also FIG. 4A-FIG. 4J. FIG. 11 A shows that ATRA and ATO cooperatively reduce Pin1 and its substrate oncoproteins in PDAC cells. Primary PDAC cells (PDAC1 and PDAC2) derived from two different human patients were treated with ATRA, ATO, or their combination (Pin1i−1) for 72 hrs, followed by examining Pin1 and its substrate oncoproteins using IB. ATRA and ATO were used in a 10:1 ratio as Pin1i−1 and only ATRA concentrations were shown. (FIG. 11B and FIG. 11C) Pin1i−1, Pin1 KD or Pin1 KO reduces many Pin1 substrates in oncogenic Kras signaling networks in PDAC cells. Primary PDAC cells were treated with Pin1i−1 (ATRA+ATO) for 72 hrs or subjected to Pin1 KD or KO (FIG. 11C) followed by examining Pin1 and its substrate oncoproteins in Kras networks using IB. ATRA and ATO were used in a 10:1 ratio as Pin1i−1 and only ATRA concentrations are shown (FIG. 11B). FIG. 11D-FIG. 11F show that ATRA, ATO, Pin1i−1 (ATRA+ATO) and Pin1i−2 (sulfopin) dose-dependently suppress cell growth and ablate Pin1. PDAC cells were treated with ATRA, ATO, Pin1i−1 or Pin1i−2 for 72 hrs, followed by examining cell growth using proliferation assay (FIG. 11D, FIG. 11F) and Pin1 using IB (FIG. 11E). ATRA and ATO were used in a 10:1 ratio as Pin1i−1 and only ATRA concentrations are shown (FIG. 11E). FIG. 11G shows that Pin1 KD inhibits human PDAC organoid growth. Pin1 KD or vector human PDAC cancer cells from two different patients (PDAC1 and PDAC2) were subjected to 3D organoid cultures, followed by assaying organoid growth curve. FIG. 11H shows that Pin1i−2 fails to suppress Pin1 KO cell growth. WT or Pin1-KO PDAC2 cells were treated with Pin1i−2 for 72 hrs, followed by examining cell growth using proliferation assay (FIG. 11H). FIG. 11I-FIG. 11L show that Pin1 inhibitors enhance the ability of GEM to inhibit human PDAC organoid growth. PDAC organoids were treated with Pin1i−1 or Pin1i−2 and/or GEM at various concentrations for 7 days, followed by examining organoid growth using microscope (FIG. 11I, FIG. 11J; PDAC2 organoids) and H&E staining, and organoid proliferation using double IF for Pan-Keratin and Ki67 IF (FIG. 11K, FIG. 11L; PDAC1 organoids, Pin1i−1 10 μM and GEM 10 nM). Scale bars, 100 μm (FIG. 11G, FIG. 11I) and 100 and 20 μm (inset) (FIG. 11K). Error bars represent mean±s.d.; *p<0.05, **p<0.001, ***p=0.0001, ****p<0.0001; n.s., not significant; by unpaired two-sided Student's t test (FIG. 11G) or one-way ANOVA for multiple comparisons (FIG. 11H, FIG. 11 J, FIG. 11L).

FIG. 12A-FIG. 12K is a set of images, dot plots, and immunoblots showing that Pin1 promotes GEM resistance and reduces the expression of PD-L1 and ENT1 at the cell surface of PDAC cells. FIG. 12A is a set of images and a dot plot that show Pin1 inhibition effects cell surface PD-L1 in PDTX mice. FIG. 12B is a set of images and a dot plot that show Pin1 inhibition effects cell surface ENT1 in PDTX mice. FIG. 12C is a set of images that shows Pin1 overexpression is correlated with reduced PD-L1 levels in human PDAC tissues. FIG. 12D is a set of images that shows Pin1 overexpression is correlated with reduced ENT1 levels in human PDAC tissues. FIG. 12E is a tabulation that shows Pin1 staining levels in human PDAC tissues. FIG. 12F is three dot plots that show Pin1 inhibition suppresses mRNA expression of Pin1, ENT1 and PD-L1 in PDAC cells. FIG. 12G is an immunoblot showing that both PD-L1 and ENT levels are regulated by lysosome-dependent proteolysis. FIG. 12H is a schematic representation of the structural domains of HIP1R and the location of only one putative Pin1 recognition Ser929-Pro motif that is identical in humans and mice. FIG. 12I is an immunoblot showing the degradation of HIP1R in HIP1R WT or S929A PDAC cells. FIG. 12J is a set of images and a dot plot that show Pin1 KO and HIP1RS929A synergistic effect with GEM to induce human PDAC organoid apoptosis. FIG. 12K is a set of images and a dot plot that show Pin1 KO and HIP1RS929A synergistic effects.

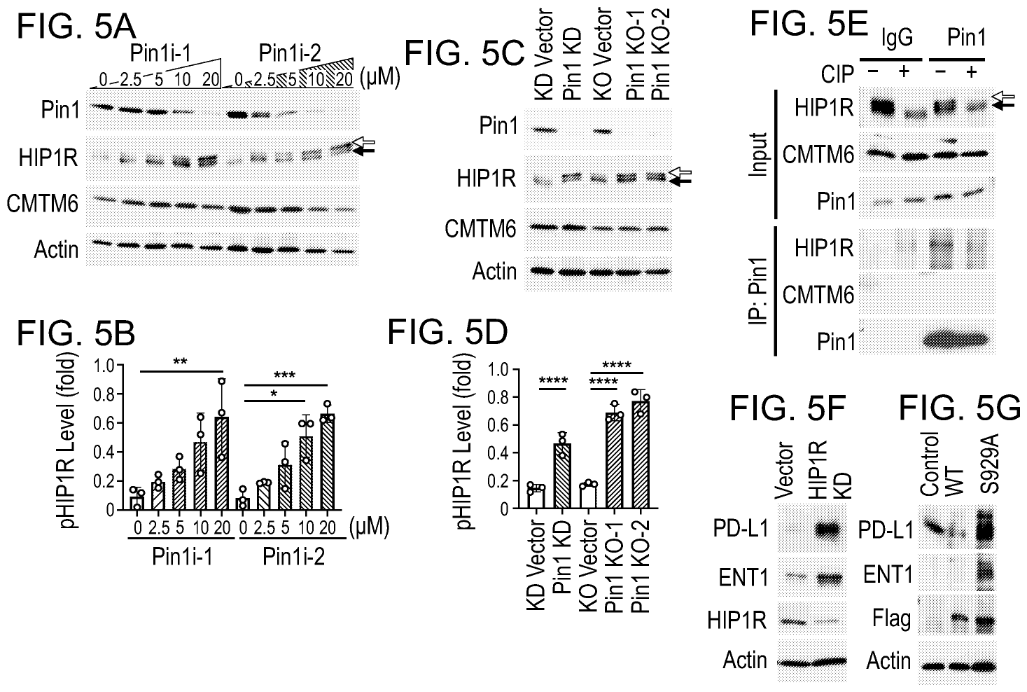

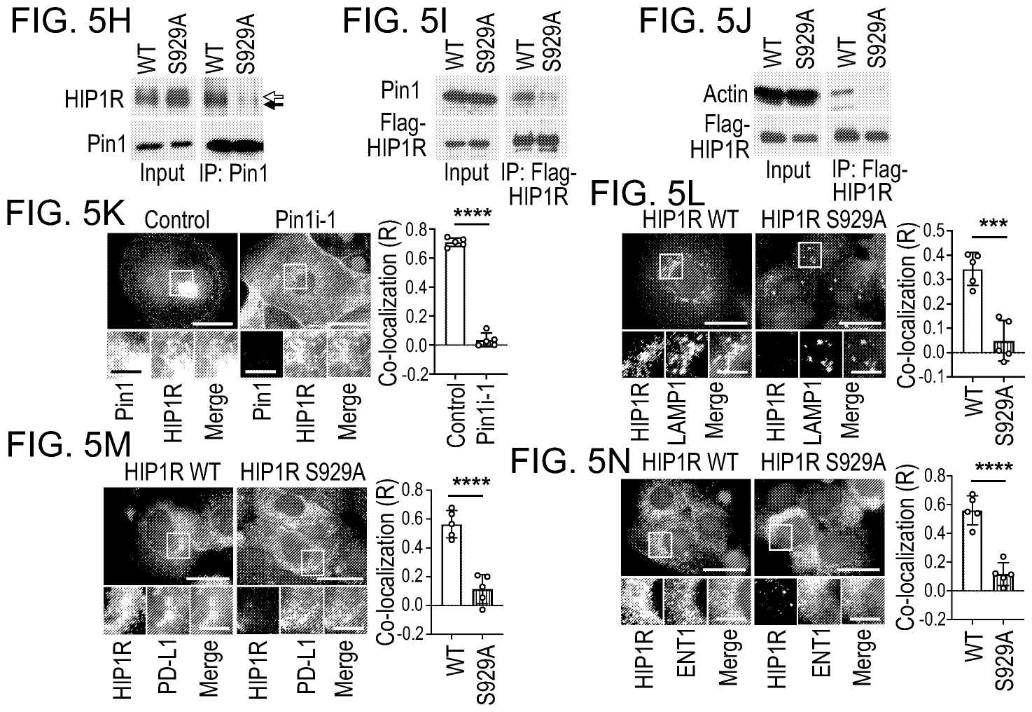

FIG. 12A-FIG. 12K show that Pin1 promotes GEM resistance and reduces the expression of PD-L1 and ENT1 at the cell surface of PDAC cells. See also FIG. 4A-FIG. 4J, FIG. 5A-FIG. 5N, and FIG. 6A-FIG. 6N. FIG. 12A and FIG. 12B show that Pin1 inhibition increases the expression of PD-L1 or ENT1 notably at the cell surface in PDTX mice. PDTX mice were treated with vehicle, Pin1i−1, or Pin1i−2 for 4 weeks, followed by IF for Pin1, PD-L1, and DAPI (FIG. 12A), or Pin1, ENT1, and DAPI (FIG. 12B) (n=5). White arrows point to PD-L1 (FIG. 12A) and ENT1 (FIG. 12B) at the cell surface. FIG. 12C-FIG. 12E show that Pin1 overexpression is correlated with reduced PD-L1 or ENT1 levels in human PDAC tissues. Human PDAC tissues were subjected to IHC for Pin1 and PD-L1 (n=53) (FIG. 12C), or for Pin1 and ENT1 (n=46) (FIG. 12D), followed by semi-quantifying their expression as high or low and examining their correlations by Pearson's chi-square test (PD-Li: p=0.013, ENT1: p=0.008) (FIG. 12E). FIG. 12F shows that Pin1 inhibition suppresses mRNA expression of Pin1, ENT1 and PD-L1 in PDAC cells. PDAC cells were treated with vehicle, Pin1i−1 (10 mM) or Pin1i−2 (5 mM) for 72 hrs, followed by RT-PCR. FIG. 12G shows that both PD-L1 and ENT levels are regulated by the lysosome-dependent proteolysis. PDAC2 cells were treated with 3-MA (15 mM), bafilomycin Al (30 nM), chloroquine (20 μM), MHLN4929 (1 μM), or MG132 (5 μM) for 12 hrs, followed by analyzing PD-L1 and ENT1 levels using IB. FIG. 12H is a schematic for the structural domains of HIP1R and the location of only one putative Pin1 recognition Ser929-Pro motif that is identical in humans and mice. FIG. 12I for the cycloheximide (CHX) chase assay shows the degradation of HIP1R in HIP1R WT or S929A PDAC cells. FIG. 12J-FIG. 12K show that Pin1 KO or HIP1RS929A synergizes with GEM or αPD1 to induce human PDAC organoid apoptosis as well as Pin1i. Pin1i-pretreated established organoids or Pin1 KO or HIP1RS929A PDAC2 organoids were treated with GEM, followed by examining apoptotic organoids using time lapse imaging and green fluorescent caspase 3/7 reagent for 48 hrs. (Pin1i−2 at 5 μM and GEM at 25 nM) (FIG. 12J). Pin1i-pretreated established organoids or Pin1 KO or HIP1RS929A PDAC2 organoids were co-cultured with activated human PBMCs, and then treated with αPD1, followed by examining apoptotic organoids using time lapse imaging and green fluorescent caspase 3/7 reagent for 40 hrs. (Pin1i−2 at 5 μM and αPD1 at 200 μg/mL) (FIG. 12K). Scale bars 50 μm and 12.5 μm (inset) (FIG. 12A, FIG. 12B), 500 μm (FIG. 12C, FIG. 12D) and 100 μm (FIG. 12J, FIG. 12K). Error bars represent mean±s.d.; *p<0.05, **p<0.001, ***p=0.0001, ****p<0.0001; n.s., not significant; by unpaired two-sided Student's t test (FIG. 12A, FIG. 12B), or one-way ANOVA for multiple comparisons (FIG. 12F, FIG. 12J, FIG. 12K).

FIG. 13A-FIG. 13O is a set of heat maps, images, and dot plots showing that targeting Pin1 synergizes with immunochemotherapy to induce human PDAC organoid apoptosis. FIG. 13A is a heat map that shows Pin1 inhibitors synergize with GEM to induce human PDAC organoid apoptosis. FIG. 13B is a set of images that show the synergic effect of Pin1i and GEM on ENT1 KD impairment. FIG. 13C is a dot plot that quantifies the percentage of apoptotic organoid cells in FIG. 13B. FIG. 13D is a heat map of that shows Pin1 inhibitors synergize with αPD1 to induce human PDAC organoid apoptosis. FIG. 13E is a set of images that show the synergic effect of Pin1i and αPD1 on PD-L1 KD impairment. FIG. 13F is a dot plot that quantifies the percentage of apoptotic organoid cells in FIG. 13E. FIG. 13G is a set of images that show the synergic effect of Pin1 inhibitors with 5-FU to induce human PDAC organoid apoptosis. FIG. 13H is a dot plot that quantifies the percentage of apoptotic organoid cells in FIG. 13G. FIG. 13I is a set of images that show the synergic effect of Pin1 inhibitors with αPDL1 to induce human PDAC organoid apoptosis. FIG. 13J is a dot plot that quantifies the percentage of apoptotic organoid cells in FIG. 13I. FIG. 13K is a set of images showing that Pin1 inhibitors synergize with immunochemotherapy (GEM+αPD1) to induce human PDAC organoid apoptosis. FIG. 13L is a dot plot showing the percentage of apoptotic organoid cells. FIG. 13M is three images that show KPC tumor-bearing mouse derived CD8+ T-cells induce KPC organoid apoptosis. FIG. 13N is a set of images that show the synergic effect of Pin1 inhibitors with αPD1 to induce KPC PDAC organoid apoptosis. FIG. 13O is a dot plot showing the percentage of apoptotic organoid cells.

FIG. 13A-FIG. 13O show that targeting Pin1 synergizes with immunochemotherapy to induce human PDAC organoid apoptosis. See also FIG. 6A-FIG. 6N. FIG. 13A shows that Pin1 inhibitors synergize with GEM to induce human PDAC organoid apoptosis. Pin1i-pretreated established organoids were treated with GEM, followed by examining organoid apoptosis for 24 hrs at different concentrations and analyzing synergy score of Pin1i−2 and GEM using Synergy finder. FIG. 13B-FIG. 13C show that ENT1 KD impairs the synergic effect of Pin1i and GEM. Control (DMSO) or Pin1i-pretreated established ENT1 WT or KD organoids were treated with control (PBS) or GEM, followed by examining apoptotic organoids using time lapse imaging and green fluorescent caspase 3/7 reagent for 24 hrs. (Pin1i−2 at 5 μM and GEM at 25 nM). FIG. 13D shows that Pin1 inhibitors synergize with αPD1 to induce human PDAC organoid apoptosis. Pin1i−pretreated established organoids were co-cultured with activated human PBMCs, and then treated with αPD1, followed by examining organoid apoptosis for 40 hrs at different concentrations and analyzing synergy score of Pin1i−2 and αPD1 using Synergy finder. FIG. 13E-FIG. 13F show that PD-L1 KD impaired the synergic effect of Pin1i and αPD1. Control (DMSO) or Pin1i−pretreated established PD-L1 WT or KD organoids were co-cultured with activated human PBMCs, and then treated with control (IgG) or αPD1, followed by examining apoptotic organoids using time lapse imaging and green fluorescent caspase 3/7 reagent for 40 hrs. (Pin1i−2 at 5 μM and αPD1 at 200 μg/mL). FIG. 13G-FIG. 13J show that Pin1 inhibitors synergize with 5-FU or αPD1 to induce human PDAC organoid apoptosis. Pin1i-pretreated organoids were treated with control (DMSO) or 5-FU at 25 nM, followed by examining PDAC organoid apoptosis using time lapse imaging for 24 hrs (FIG. 13G, FIG. 13H), or were co-cultured with activated human PBMCs, and then treated with control (IgG) or αPD-L1 at 200 μg/mL, followed by examining PDAC organoid apoptosis using time lapse imaging for 40 hrs (FIG. 13I, FIG. 13J). FIG. 13K-FIG. 13L show that Pin1 inhibitors synergize with immunochemotherapy (GEM+αPD1) to induce human PDAC organoid apoptosis. Pin1i-pretreated organoids were co-cultured with activated human PBMCs, and then treated with control (PBS and/or IgG), GEM and/or 0PD1, followed by assaying PDAC organoid apoptosis for different times at constant concentrations (Pin1i−1 at 10 μM, Pin1i−2 at 5 μM, GEM at 10 nM, and αPD1 at 100 μg/mL). FIG. 13M shows that KPC tumor-bearing mouse derived CD8+ T-cells induce KPC organoid apoptosis. KPC tumor derived organoids were co-cultured with/without activated CD8+ T-cells derived from KPC tumor-bearing mice or their tumor-free controls, followed by examining apoptotic organoids using time lapse imaging and green fluorescent caspase 3/7 reagent for 40 hrs. FIG. 13N-FIG. 13O show that Pin1 inhibitors synergize with αPD1 to induce KPC PDAC organoid apoptosis. Control (DMSO) or Pin1i−2 pre-treated KPC tumor derived organoids were co-cultured with the KPC tumor-bearing mouse derived activated CD8+ T-cells, and then treated with control (PBS+IgG), or GEM and 0PD1, followed by examining apoptotic organoids using time lapse imaging and green fluorescent caspase 3/7 reagent for 24 hrs (Pin1i−2 at 5 μM and αPD1 at 200 μg/mL). Scale bars 100 μm (FIG. 13B, FIG. 13E, FIG. 13G, FIG. 13I, FIG. 13K, FIG. 13M, FIG. 13N). Error bars represent mean±s.d.; *p<0.05, **p<0.001, ***p=0.0001, ****p<0.0001; n.s., not significant; by unpaired two-sided Student's t test (FIG. 13L), or by one-way ANOVA for multiple comparisons (FIG. 13C, FIG. 13F, FIG. 13H, FIG. 13J, FIG. 13O).

FIG. 14A-FIG. 14G is a set of images, dot plots, and graphical representations showing that targeting Pin1 renders primary PDAC tumors eradicable by immunochemotherapy in genetically engineered KPC transgenic mice. FIG. 14A is a dot plot that shows Pin1 inhibitors disrupt αSMA in KPC mice. FIG. 14B is a set of images that show Pin1 inhibitors in KPC mice. FIG. 14C is a set of images and a dot plot that show Pin1 inhibitors increased PD-L1 expression in KPC mice. FIG. 14D is a set of images and a dot plot that show Pin1 inhibitors increased ENT1 expression in KPC mice. FIG. 14E is a set of images that show Pin1 inhibitors render PDAC tumors eradicable by immunochemotherapy in KPC mice. FIG. 14F is a set of images that show liver metastasis. FIG. 14G is two graphical representations that show Pin1 method of action cancer and stromal cells.

FIG. 14A-FIG. 14G show that targeting Pin1 renders primary PDAC tumors eradicable by immunochemotherapy in genetically engineered KPC transgenic mice. See also FIG. 7A-FIG. 7G. FIG. 14A-FIG. 14B show that Pin1 inhibitors disrupt the desmoplastic and immunosuppressive TME in KPC mice. Overt tumor-bearing KPC mice were treated with vehicle or G+P with or without Pin1i−1 or Pin1i−2, followed by assaying CAF activation using αSMA IF as in FIG. 14A or PDGFRα IF as in FIG. 14B and CAF proliferation using double IF for PDGFRα and Ki67 as in FIG. 14B, (n=5). FIG. 14C-FIG. 14D show that Pin1 inhibitors increased PD-L1 and ENT1 expression in KPC mice. Overt tumor-bearing KPC mice were treated with vehicle or G+P with/without Pin1i−1 or Pin1i−2, followed by assaying PD-L1 expression as in FIG. 14C and ENT1 expression as in FIG. 14D using IF after autopsy (n=5). FIG. 14E-FIG. 14F show that Pin1 inhibitors render PDAC tumors eradicable by immunochemotherapy in KPC mice. Overt tumor-bearing KPC mice were treated with vehicle or G+P with or without Pin1i−1 or Pin1i−2, for up to 180 days, followed by detecting macroscopic and microscopic tumors as in FIG. 14E, and liver metastasis as in FIG. 14F after autopsy (n=10). FIG. 14G, left hand side, is a cartoon representation that shows that in PDAC, Pin1 acts on cancer cells to induce HIP1R-mediated endocytosis and lysosomal degradation of PD-L1 and ENT1, and on stromal cells such as CAFs to drive the desmoplastic and immunosuppressive TME, as well as activating multiple oncogenic pathways, including many in oncogenic Kras signaling in both cells, thereby inducing drug resistance to immunochemotherapy. FIG. 14G, right hand side, is a cartoon representation that shows that Pin1 inhibitors suppress multiple oncogenic pathways, increase the cell surface expression of PD-L1 and ENT1 in cancer and disrupt the desmoplastic and immunosuppressive TME, together rendering aggressive PDAC tumors eradicable by immunochemotherapy (GEM+αPD1) in a synergistic manner. Scale bars 100 μm and 50 μm for FIG. 14B, 100 μm for FIG. 14C and FIG. 14D, 5000 μm and 200 μm for FIG. 14E, and 200 μm in FIG. 14F. Error bars represent mean±s.d.; *p<0.05, **p<0.001, ***p=0.0001, ****p<0.0001; n.s., not significant; by one-way ANOVA for multiple comparisons for FIG. 14A-FIG. 14D.

FIG. 15 is a graphic representation showing several elements for aggressive, treatable, and eradicable PDAC.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

### Pancreatic Ductal Adenocarcinoma (PDAC)

Pancreatic ductal adenocarcinoma (PDAC) is an extremely dismal malignancy, with a mortality rate almost equal to its incidence. PDAC does not respond well to current chemotherapies, targeted therapies, or immunotherapies, being projected to be the second leading cause of cancer deaths by 2030, due in part to inherent intratumor heterogeneity and uniquely desmoplastic and immunosuppressive tumor microenvironment (TME). A continuous crosstalk between cancer cells and TME increases tumor malignancy and drug resistance. Identification of the regulation of the desmoplastic and immunosuppressive TME and their interactions with tumor cells would not only offer new insight into the development of PDAC but also might overcome its resistance to the current cancer therapies.

A central common signaling mechanism in cell proliferation and transformation is proline-directed phosphorylation regulating numerous oncoproteins and tumor suppressors. The function of many of these phosphoproteins is further regulated by a unique cis-trans proline isomerase, Pin1, with its aberration contributing to disease. Notably in cancer, Pin1 overactivation promotes tumorigenesis by activating >60 oncoproteins and inactivating >30 tumor suppressors. Moreover, Pin1 knockout mice have no overt phenotype for half lifespan but are highly resistant to tumorigenesis induced by oncogenes or tumor suppressors. Thus, targeting Pin1 might simultaneously block multiple cancer-driving pathways, for example, as supported by the recent identification of Pin1 inhibitors, including approved leukemia drugs, which have demonstrated global impact. However, almost all Pin1 studies have been performed on cancer cells, including for PDAC, and little is known about the role in TME. Prior to the invention described herein, nothing was known about role of Pin1 in cancer immunotherapy.

In human PDAC patients, Pin1 overexpression in cancer cells and its correlation with poor overall survival has been confirmed. Pin1 is overexpressed in tumor stromal cells called cancer-associated fibroblasts (CAFs) and its overexpression is correlated with poor overall survival, especially when Pin1 is overexpressed both in cancer cells and CAFs, with a striking five-fold difference in overall patient survival. GEM is the clinically proven chemotherapy, but its therapeutic effects are very limited. Checkpoint immunotherapies or targeted therapies also have little effect on PDAC.

### Cancer Associated Fibroblast (CAF)

In PDAC, stromal CAFs play a vital role in promoting the desmoplastic and immunosuppressive TME, as well as tumor growth and malignancy, and have emerged as interesting cancer targets (Ho et al., Nat Rev Clin Oncol 17, 527-540 (2020); Hosein et al., Nat Rev Gastroenterol Hepatol 17, 487-505 (2020); Neesse et al., Gut (2018); Whittle and Hingorani, Gastroenterology 156, 2085-2096 (2019)). However, the mechanisms controlling CAF activation and function are still not fully understood. Of note, CAFs are heterogenous and their functions are complex in PDAC (Hosein et al., Nat Rev Gastroenterol Hepatol 17, 487-505 (2020)).

### Targeting Immune Checkpoints

Targeting immune checkpoints such as the one mediated by programmed cell death protein 1 (PD-1) and its ligand PD-L1 has improved patient survival in various cancers (Gotwals et al., Nat Rev Cancer 17, 286-301 (2017); Mahoney et al., Nat Rev Drug Discov 14, 561-584 (2015); Sharma and Allison, Science 348, 5661 (2015); Zou et al., Science translational medicine 8, 328rv324 (2016)). However, the response rate is low in PDAC patients (Brahmer et al., N Engl J Med 366, 2455-2465 (2012); Royal et al., J Immunother 33, 828-833 (2010)) due to diminished tumor immunogenicity, including low PD-L1 expression and the immunosuppressive TME, but the underlying mechanisms are not well understood (Gotwals et al., Nat Rev Cancer 17, 286-301 (2017); Mahoney et al., Nat Rev Drug Discov 14, 561-584 (2015); Sharma and Allison, Science 348, 5661 (2015); Zou et al., Science translational medicine 8, 328rv324 (2016)). PD-L1 expression is tightly controlled at the transcriptional and post-translational levels, but is aberrantly altered in human cancers (Burr et al., Nature 549, 101-105(2017); Casey et al., Science 352, 227-231 (2016); Cha et al., Mol Cell 76, 359-370 (2019); Dorand et al., Science 353, 399-403 (2016); Lim et al., Cancer Cell 30, 925-939 (2016); Zhang et al., Nature 553, 91-95 (2018)). Importantly, although PD-L1 has been well studied for its engagement with PD-1 on T-cells to evade antitumor immunity (Gotwals et al., Nat Rev Cancer 17, 286-301 (2017); Mahoney et al., Nat Rev Drug Discov 14, 561-584 (2015); Sharma and Allison, Science 348, 5661 (2015); Zou et al., Science translational medicine 8, 328rv324 (2016)), recent studies have shown that the presence of PD-L1-expressing cancer cells within tumors is known to be an important predictor of response to immune checkpoint blockade (ICB) in patients (Ansell et al., N Engl J Med 372, 311-319 (2015); Galluzzi et al., Science translational medicine 10, eaat7807 (2018); Herbst et al., Nature 515, 563-567 (2014)). Moreover, upregulating PD-L1 expression in cancer cells using different approaches improves ICB efficacy in experimental models (Deng et al., J Clin Invest 124, 687-695 (2014); Herter-Sprie et al., JCI Insight 1, e87415 (2016); Jiao et al., Clin Cancer Res 23, 3711-3720 (2017); Zhang et al., Nature 553, 91-95 (2018)). Since most PDAC tumors are negative for PD-L1 (Liang et al., Diagnostic pathology 13, 5 (2018); Tessier-Cloutier et al., BMC Cancer 17, 618 (2017)), it is critical to understand mechanisms and signaling pathways behind the regulation of PD-L1 levels to improve ICB response and efficacy.

### PD-L1 Expression Levels

Recent findings have shown that elevated PD-L1 expression levels tend to respond better to PD-1 blockade in human cancer patients (Ansell et al., N Engl J Med 372, 311-319 (2015); Galluzzi et al., Science translational medicine 10, eaat7807 (2018); Herbst et al., Nature 515, 563-567 (2014)). Further, induction of PD-L1 expression in cancer cells in response to radiation (Deng et al., J Clin Invest 124, 687-695 (2014); Herter-Sprie et al., JCI Insight 1, e87415 (2016)), PARP inhibitors (Jiao et al., Clin Cancer Res 23, 3711-3720 (2017)) or inhibition of PD-L1 proteasomal proteolysis using CDK4/6 inhibitors (Zhang et al., Nature 553, 91-95 (2018)) potentiates αPD1 efficacy.

Protein degradation is a key mechanism to regulate not only numerous oncogenic proteins (Lu and Hunter, Cell Res 24, 1033-1049 (2014); Zhou and Lu, Nat Rev Cancer 16, 463-478 (2016)), but also many cancer therapeutic targets/receptors/biomarkers, including PD-L1 (Burr et al., Nature 549, 101-105 (2017); Mezzadra et al., Nature 549, 106-110 (2017); Wang et al., Nat Chem Biol 15, 42-50 (2019); Zhang et al., Nature 553, 91-95 (2018)), and ENT1 (Hu et al., Oncol Rep 38, 2069-2077 (2017)). Notably, HIP1R is a key protein in lysosomal proteolysis by binding with a membrane protein such as PD-L1 and cytoplasmic actin for endocytosis (Gottfried et al., Biochem Soc Trans 38, 187-191 (2010); Messa et al., eLife 3, e03311 (2014); Wang et al., Nat Chem Biol 15, 42-50 (2019)). However, prior to the invention described herein, whether this PD-L1 degradation was further regulated was not known, which is especially important for PDAC because the majority of PDAC patients have very low PD-L1 (Liang et al., Diagnostic pathology 13, 5 (2018); Tessier-Cloutier et al., BMC Cancer 17, 618 (2017)).

### Pin1

A central common signaling mechanism in cancer is proline-directed phosphorylation regulating numerous oncoproteins and tumor suppressors (Blume-Jensen and Hunter, Nature 411, 355-365 (2001); Ubersax and Ferrell, Nat Rev Mol Cell Biol 8, 530-541 (2007)), many of which are further regulated by a unique proline isomerase, Pin1 (Lu and Hunter, Cell Res 24, 1033-1049 (2014); Zhou and Lu, Nat Rev Cancer 16, 463-478 (2016)). Aberrant Pin1 overactivation promotes tumorigenesis by activating over 60 oncoproteins and inactivating over 30 tumor suppressors in various cancers, including numerous substrates in oncogenic Kras signaling (Lu and Hunter, Cell Res 24, 1033-1049 (2014); Zhou and Lu, Nat Rev Cancer 16, 463-478 (2016)), which is dominant in PDAC (Eser et al., Br J Cancer 111, 817-822 (2014); Waters and Der, Cold Spring Harbor perspectives in medicine 8, a031435 (2018)). Furthermore, PIN1−/− mice develop normally and have no major phenotype for an extended period of time (Fujimori et al., Biochem Biophys Res Commun 265, 658-663 (1999); Liou et al., Proc Natl Acad Sci USA 99, 1335-1340 (2002)), but are highly resistant to tumorigenesis induced by transgenic overexpression of oncogenes or loss of tumor suppressors (Girardini et al., Cancer Cell 20, 79-91 (2011); Liao et al., Mol Cell 68, 134-1146 (2017); Takahashi et al., Oncogene 26, 3835-3845 (2007); Wulf et al., Nature 581, 100-105 (2004); Zhou and Lu, Nat Rev Cancer 16, 463-478 (2016)). Moreover, genetic polymorphisms that reduce Pin1 expression are also associated with reduced risk for multiple cancers in humans (Li et al., PLoS One 8, e68148 (2013)). These data suggest that targeting Pin1 in PDAC might simultaneously block multiple oncogenic signaling pathways without major toxicity (Lu and Hunter, Cell Res 24, 1033-1049 (2014); Zhou and Lu, Nat Rev Cancer 16, 463-478 (2016)).

This notion has been corroborated by the recent unexpected identification of all-trans retinoic acid and arsenic trioxide (ATRA+ATO) as synergistic Pin1 inhibitors that block multiple cancer-driving pathways, eliminate cancer stem cells, and increase response to chemotherapy, targeted therapy, and radiation in various cancers (Kozono et al., Nature communications 9, 3069 (2018); Liu et al., Nat Cell Biol 21, 203-213 (2019); Luo et al., Cancer Res 80, 3033-3045 (2020); Mugoni et al., Cell Res 29, 446-459 (2019); Wang et al., Cancer letters 444, 8293 (2019); Wei et al., Nature Med 21, 457-466 (2015); Zhou and Lu, Nat Rev Cancer 16, 463-478 (2016)). These findings offer a molecular insight into how ATRA+ATO synergistically induce protein degradation of the disease-causing the fusion oncogene PML-RARa (a Pin1 substrate) and safely eradicate deadly acute promyelocytic leukemia (APL) (de The and Chen, Nat Rev Cancer 10, 775-783 (2010); Kozono et al., Nature communications 9, 3069 (2018); Wang and Chen, Blood 111, 2505-2515 (2008); Wei et al., Nature Med 21, 457-466 (2015); Zhou and Lu, Nat Rev Cancer 16, 463-478 (2016)). However, as almost all Pin1 studies in cancer focus on cancer cells, prior to the invention described herein, it was unknown whether Pin1 has any role in the TME and cancer immunotherapy and whether Pin1 inhibitors could render a solid malignancy eradicable.

Pin1-catalyzed prolyl isomerization regulates the functions of its substrates through multiple different mechanisms, including controlling catalytic activity, turnover, phosphorylation, interactions with DNA, RNA or other proteins, and subcellular localization and processing. Pin1 is tightly regulated normally and its deregulation can have a major impact on the development and treatment of cancer and neurodegenerative diseases.

Pin1 substrates comprise proteins involved in signal transduction, including RAF1, HER2, eNOS, SMAD2/3, Notch1, Notch3, AKT, FAK, P7013K, PTP-PEST, MEK1, GRK2, CDK10, FBXW7, PIP4Ks, PKM2 and JNK1; proteins involved in gene transcription including SIN3-RPD3, JUN, β-catenin, CF-2, hSPT5, MYC, NF-κB, FOS, RARα, SRC-3/AIB1, STAT3, MYB, SMRT, FOXO4, KSRP, SF-1, Nanog, PML, Mutant p53, ΔNp63, Oct4, ERα, PKM2, AR, SUV39H1, RUNX3, KLF10, Osterix and PML-RARα; proteins involved in cell cycle at the G1/S including Cyclin D1, KI67, Cyclin E, p27, LSF and RB1; proteins involved in cell cycle at the G2/M and M including NIMA, RAB4, CDC25, WEE1, PLK1, MYT1, CDC27, CENP-F, INCENP, RPB1, NHERF-1, KRMP1, CK2, TOPIIa, DAB2, p54NRB, SIL, EMIl, CEP55, BORA, Survivin, SEPT9, SP1, SWI6, WHI5 and Separase; proteins involved in DNA damage/stress response and apoptosis including p53, BCL-2, p73, BIMEL, p66SHC, DAXX, MCL-1, NUR77, HIPK2, RBBP8, p63, HSF1, HIF-la, CHE-1 and PGK1; proteins involved in immune response including NFAT, AUF1, IRF3, BTK, BAX, COX-2, p47PHOX, IRAK1, GR and FADD; proteins involved in viral or parasitic infection and transformation including HBX, A3G, v-Rel, Tax, Capsid protein, Integrase, BALF5, RTA, FBXW7 and ORF1p; proteins involved in neuronal survival and degeneration including TAU, APP, Synphilin-1, Gephyrin, mGluR5, REST, GRO/TLE1 and CRMP2A. (Zhou and Lu, “The isomerase Pin1 controls numerous cancer-driving pathways and is a unique drug target” Nature Reviews Cancer 16:463-478; Supplementary Information (2016)).

### Pin1 Overexpression in PDAC

Herein is disclosed that Pin1 overexpression in PDAC drives resistance to chemotherapy and checkpoint immunotherapy not only by promoting the fibrotic and immunosuppressive TME, but also by inducing lysosomal degradation of PD-L1 and ENT1, which are a therapeutic response markers for cancer immunotherapy and chemotherapy. Moreover, the inhibition of Pin1 using either the approved leukemia drugs all-trans retinoic acid and arsenic trioxide (ATRA+ATO) (Pin1i−1) or a newly discovered highly specific Pin1 covalent inhibitor (sulfopin) (Pin1i−2) eradicates most PDAC by synergizing with immunotherapy and chemotherapy in various preclinical models in vitro and in vivo.

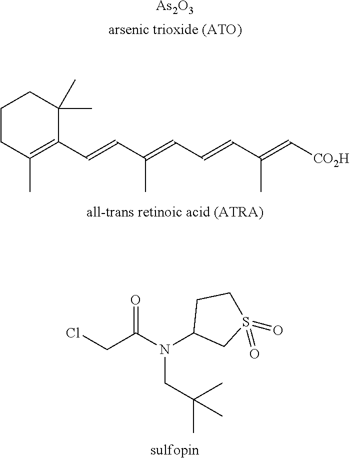 [SMILES: "As2O3" ; CC1=C(/C=C/C(C)=C/C=C/C(C)=C/C(=O)O)C(C)(C)CCC1 ; CC(C)(C)CN(C(=O)CCl)C1CCS(=O)(=O)C1]

To determine the function of Pin1 overexpression in CAFs, primary human CAFs were isolated from cancer tissues in PDAC patients and their Pin1 function was inhibited resulting in the finding that Pin1 chemical inhibitors, genetic knockdown (KD) or CRISPR knockout (KO) suppresses CAF proliferation, induces CAF quiescence and inhibits CAF cytokine production. Importantly, Pin1-inhibited or KO human primary CAFs fail to promote cancer cell growth and invasion as demonstrated using indirect and direct co-culture experiments of 3-dimensional (3D) human PDAC organoids. Moreover, Pin1 CRISPR KO human primary CAFs also fail to promote the fibrotic TME and tumor growth when co-transplanted with human PDAC cancer cells into pancreatic tissues in immunocompromised NSG mice. These results demonstrate that Pin1 overexpression acts on TME cell such as CAFs to promote the fibrotic TME and tumor growth in PDAC.

In some embodiments, the one or more Pin1 inhibitors suppresses CAF proliferation. In some embodiments, the one or more Pin1 inhibitors induces CAF quiescence. In some embodiments, the one or more Pin1 inhibitors inhibits CAF cytokine production.

To further confirm that Pin1 plays a major role in driving TME, PDAC cancer cells were isolated from a KPC (LSL-K-RasG12D/+; LSL-p53R172H/+; Pdx1-Cre) mouse model of human PDAC and then transplanted into pancreatic tissues in syngeneic WT B6 immunocompetent mice. When tumors reached 0.5 cm diameter, they were treated with two different Pin1 inhibitors for 1 month. Both Pin1i−1 and Pin1i−2 potently reduce the fibrotic TME. Moreover, it was discovered that Pin1 inhibitors increase immune killing CD8α+ CTLs and reduce immunosuppressive Fox3+ T-reg and Ly6g+CD11b+ MDSCs, suggesting that Pin1 inhibition might disrupt immunosuppressive TME and render PDAC responsive to checkpoint immunotherapy. To examine this possibility, KPC mouse-derived orthotopic allografts were treated with two different Pin1 inhibitors, gemcitabine (GEM)+anti-PD-1, Pin1i+anti-PD-1 or Pin1i+GEM+anti-PD-1. GEM+anti-PD-1 increased overall survival a little, as shown before and both Pin1i−1 and Pin1i−2 had a slightly bigger effect. Importantly, Pin1i+anti-PD-1 dramatically reduces tumor growth and increases overall survival. Most strikingly, Pin1i+GEM+anti-PD-1 combination leads to tumor shrinking, with 87.5% survival for over 1 year, even though the treatment was stopped after 120 days. For these surviving mice, there was neither macroscopic nor microscopic PDAC. These results demonstrate that Pin1 inhibitors not only disrupt the fibrotic and immunosuppressive TME, but also eradicate most PDAC by synergizing with immunotherapy and chemotherapy.

In some embodiments, the one or more Pin1 inhibitors reduces the fibrotic TME. In some embodiments, the one or more Pin1 inhibitors increases the level of immune killing CD8α+ CTLs. In some embodiments, the one or more Pin1 inhibitors reduces the level of immunosuppressive Fox3+ T-reg. In some embodiments, the one or more Pin1 inhibitors reduces the level of Ly6g+ CD11b+ MDSCs. In some embodiments, the one or more Pin1 inhibitors disrupt the immunosuppressive TME. In some embodiments, the one or more Pin1 inhibitors render PDAC responsive to checkpoint therapy. In some embodiments, the combination of one or more Pin1 inhibitors and anti-PD-1 reduces tumor growth. In some embodiments, the combination of one or more Pin1 inhibitors and anti-PD-1 increases overall survival by, e.g., at least 5%. In some embodiments, the combination of one or more Pin1 inhibitors, GEM and anti-PD-1 results in tumor shrinking. In some embodiments, the combination of one or more Pin1 inhibitors, GEM and anti-PD-1 increases overall survival. In some embodiments, the reduction in tumor growth is sustained after the treatment is stopped.

To further demonstrate that Pin1 inhibitors are able to synergize with gemcitabine and anti-PD-1 to allow T-cells to kill human PDAC cells, human PDAC organoids were established and treated with Pin1 inhibitors, and organoids were co-cultured with activated T-cells, followed by treatment with GEM or anti-PD-1 to assay human PDAC organoid killing using caspase 3/7 live cell movies. Pin1 inhibitors dramatically increase the ability of chemotherapies (GEM, or 5-FU), or immunotherapies (anti-PD-1 or anti-PD-L1) to allow T-cells to kill human PDAC organoids and the effects are highly synergistic.

In some embodiments, the combination of one or more Pin1 inhibitors, GEM and anti-PD-1/anti-PD-L1 results in the killing of human PDAC organoids. In some embodiments, the combination of one or more Pin1 inhibitors, 5-FU, and anti-PD-1/anti-PD-L1 results in the killing of human PDAC organoids. In some embodiments, the killing is synergistic.

Finally, to demonstrate that Pin1 inhibitors are able to suppress TME and synergize with gemcitabine and anti-PD-1 to eradicate PDAC, genetically modified KPC (LSL-K-RasG12D/+; LSL-p53R172H/+; Pdx1-Cre) mice were treated with two different Pin1 inhibitors, GEM+anti-PD-1, or their combination, when their tumors reached 0.5 cm diameter. Where GEM+anti-PD-1 do not significantly affect immunosuppressive TME or increase overall survival, with most mice being dead with 3 months, Pin1i−1 or Pin1i−2 and GEM+anti-PD-1 combination not only disrupts immunosuppressive TME, but also dramatically increases overall survival, with 60 or 70% of treated mice surviving for over 6 months. For these surviving mice, there was detectable macroscopic tumors, although microscopic tumors are noted, indicating that tumors are shrinking or disappearing. These results demonstrate that Pin1 inhibitors not only disrupt the immunosuppressive TME, but also eradicate most PDAC by synergizing with immunotherapy and chemotherapy, even in a genetically modified KPC mouse model of human PDAC.

In some embodiments, the combination of one or more Pin1 inhibitors, GEM and anti-PD-1 disrupts the immunosuppressive TME. In some embodiments, the combination of one or more Pin1 inhibitors, GEM and anti-PD-1 increases overall survival.

To elucidate the molecular mechanisms underlying these strikingly synergistic effects, it has been discovered that Pin1 inhibitors, KD or CRISPR KO dramatically increase PD-L1 and ENT1 protein expression in human primary PDAC cells, organoids or KPC mice, because Pin1 interacts with HIP1R and promotes HIP1R-mediated lysosomal degradation of ENT1 and PD-Li. Since PD-L1 and ENT1 are required for immunotherapies (anti-PD-1 or anti-PD-L1) to kill tumor cells and for chemotherapies (GEM or 5-FU) to enter cancer cells, respectively, thereby acting as therapeutic response markers for cancer immunotherapy and chemotherapy, these results explain molecular mechanisms by which Pin1 inhibitors synergize with immunotherapy and chemotherapy in various preclinical models in vitro and in vivo.

In some embodiments, the one or more Pin1 inhibitors increases PD-L1 protein expression. In some embodiments, the one or more Pin1 inhibitors increases ENT1 protein expression.

In summary, it is disclosed that in PDAC, Pin1 overexpression in cancer cells as well as CAFs promotes their growth and interactions to generate the fibrotic and immunosuppressive TME as well as promotes HIP1R-mediated lysosomal degradation of ENT1 and PD-L1, together resulting in primary resistance to chemotherapy and immunotherapy. Pin1 inhibition eradicates most PDAC tumors by suppressing PDAC and CAFs growth and their interaction to produce the fibrotic and immunosuppressive TME and suppressing HIP1R-mediated lysosomal degradation of ENT1 and PD-L1, rendering PDAC responsive chemo- and immunotherapy. These results show that the combination of Pin1 inhibition+checkpoint blockage+chemotherapy can eradicate most pancreatic cancers in preclinical models. These studies indicate that Pin1 inhibitors can be used to combine with currently available immunotherapies and chemotherapies to eradicate pancreatic cancer and likely many other solid tumors. These combinations may transform solid tumor treatment like acute promyelocytic leukemia (APL) treatment.

In some embodiments is provided a method of treating a disease or disorder mediated by dysregulated Pin1 activity. In some embodiments, the disease or disorder may include, for example, skin merkel cell cancer, thyroid mudullary cancer, uterus carcinoma, liposarcoma, ovary Brenner tumor, uterus cervix squamous cell carcinoma, prostate cancer (untreated), NHL, prostate cancer (hormone-refract), lung small cell cancer, adrenal gland cancer, ovary serous cancer, oligodendroglioma, glioblastoma multiforme, lung large cell cancer, lung squamous cell carcinoma, thyroid adenoma, skin malignant melanoma, mouth cancer, ovary mucinous cancer, ovary endometroid cancer, thyroid follicular cancer, parathyroid adenocarcinoma. NHL diffuse large B, skin benign nevus, hepatocellular carcinoma, breast ductal cancer, breast lobular cancer, breast mucinous cancer, breast medullary cancer, lung adenocarcinoma, lipoma, colon adenoma sever dysplasia, astrocytoma, colon adenoma moderate dysplasia, colon adenoma mild dysplasia, thymoma, MALT lymphoma, gall bladder adenocarcinoma, esophagus adenocarcinoma, bladder transitional cell carcinoma, thyroid papillary cancer, skin squamous cell cancer, breast tubula cancer, colon adenocarcinoma, testis non-seminomatous cancer, kidney clear cell carcinoma, among others.

In some embodiments, the combination of one or more Pin1 inhibitors, checkpoint blockage, and chemotherapy may eradicate most pancreatic cancers. In some embodiments, the combination of one or more Pin1 inhibitors, GEM/5-FU, and anti-PD-1/anti-PD-L1 results in the reduction in the size of solid tumors. In some embodiments, the solid tumor is PDAC. In some embodiments, the solid tumor is breast cancer. In some embodiments, the solid tumor is CRC. In some embodiments, the combination of one or more Pin1 inhibitors, GEM/5-FU, and anti-PD-I/anti-PD-L1 results in the eradication of pancreatic cancers. In some embodiments, the Pin1 inhibitor is Pin1i−1. In some embodiments, the Pin1 inhibitor is Pin1i−2. In some embodiments, the combination of one or more Pin1 inhibitors, GEM/5-FU, and anti PD-1/anti-PD-L1 results in a synergistic reduction in the size of solid tumors. In some embodiments, the solid tumor is PDAC. In some embodiments, the solid tumor is breast cancer. In some embodiments, the solid tumor is CRC.

Herein is disclosed to combine Pin1 inhibitors with currently available immunotherapies and chemotherapies to eradicate most pancreatic cancers as well as many other solid tumors. Two different Pin1 inhibitors (the approved leukemia drugs ATRA+ATO or the highly specific Pin1 covalent inhibitor sulfopin) eradicate most PDAC by synergizing with immunotherapies and chemotherapies in various preclinical models in vitro and in vivo. As such, the invention describes the discovery that Pin1 inhibitors have the unique and promising property to eradicate most solid tumors by synergizing with the currently available immunotherapy and chemotherapy using pancreatic cancer as an example.

Herein is disclosed that Pin1 is overexpressed both in cancer cells and cancer-associated fibroblasts (CAFs) and correlates with poor survival in PDAC patients. Targeting Pin1 using clinically available drugs induces complete elimination or sustained remissions of aggressive PDAC by synergizing with anti-PD-1 and gemcitabine (GEM) or 5-FU in diverse model systems. Mechanistically, Pin1 drives the desmoplastic and immunosuppressive TME by acting on CAFs, induces lysosomal degradation of the PD-1 ligand PD-L1 and the gemcitabine transporter ENT1 in cancer cells, in addition to activating multiple cancer pathways. Thus, Pin1 inhibition simultaneously blocks multiple cancer pathways, disrupts the desmoplastic and immunosuppressive TME, and upregulates PD-L1 and ENT1, thereby rendering PDAC eradicable by immunochemotherapy.

In some embodiments, the combination of one or more Pin1 inhibitors, GEM/5-FU, and anti-PD-1/anti-PD-L1 induces complete elimination of aggressive PDAC. In some embodiments, the combination of one or more Pin1 inhibitors, GEM/5-FU and anti-PD-1/anti-PD-L1 induces sustained remission of aggressive PDAC.

Herein is reported that Pin1 drives the desmoplastic and immunosuppressive TME in PDAC by acting on CAFs and induces PD-L1 and ENT1 endocytosis and lysosomal degradation in cancer cells by acting on HIP1R, in addition to activating multiple oncogenic signaling pathways. Consequently, targeting Pin1 using ATRA+ATO simultaneously blocks multiple cancer pathways, disrupts the desmoplastic and immunosuppressive TME, and upregulates PD-L1 and ENT1, thereby rendering aggressive PDAC eradicable by synergizing with immunochemotherapy in vitro, in vivo and ex vivo. These findings may have immediate therapeutic impact on PDAC patients as some Pin1 inhibitors are approved drugs.

PDAC is notoriously resistant to current therapies due to inherent tumor heterogeneity and highly desmoplastic and immunosuppressive TME. Here it is shown that Pin1 is overexpressed both in cancer cells and CAFs in PDAC patients, and highly correlates with the desmoplastic and immunosuppressive TME and poor patient survival. Functionally, besides activating multiple cancer pathways, Pin1 drives the desmoplastic and immunosuppressive TME and promotes tumor malignancy and drug resistance by acting on stromal cells such as CAFs and inducing endocytosis and degradation of PD-L1 and ENT1 in cancer cells by acting on pS929-HIP1R. Consequently, targeting Pin1 offers a unique and promising approach to render this deadly cancer eradicable. In some embodiments is provided the design of a clinical trial using Pin1 inhibitors in combination with immunochemotherapy for PDAC patients given that the ATRA+ATO therapy is a safe modality to eradicate most APL patients.

Here it is shown that Pin1 is overexpressed in CAFs and correlates with the desmoplastic and immunosuppressive TME and poor survival. Targeting Pin1 using Pin1 inhibitors (Pin1i), KD or KO not only inhibits multiple oncogenic pathways in CAFs, but also suppresses their growth, activation, and cytokine production implicated in immunosuppression (Erkan et al., Gut 61, 172-178 (2012); Mace et al., Gut 67, 320-332 (2018); Mariathasan et al., Nature 554, 544-548 (2018)). Furthermore, targeting Pin1 eliminates the ability of CAFs to promote the desmoplastic TME, tumor growth and malignancy in human PDAC organoids and/or PDOX mice. Moreover, Pin1 inhibitors also potently increase tumor-infiltrating cytotoxic T-cells and decrease immunosuppressive cells in GDA and KPC mice. These results are consistent with the reports that ATRA reduces the desmoplastic and immunosuppressive TME, and tumor growth and malignancy via multiple cancer and CAF-related pathways (Carapuca et al., J Pathol 239, 286-296 (2016); Chen et al., Cancer Sci 110, 2442-2455 (2019); Ene-Obong et al., Gastroenterology 145, 1121-1132 (2013); Froeling et al., Gastroenterology 141, 1486-1497, 1497 e1481-1414 (2011); Guan et al., Cancer letters 345, 132-139 (2014); Kocher et al., Nature communications 11, 4841 (2020)) because many ATRA-mediated effects might be at least partially due to Pin1 inhibition (Wei et al., Nature Med 21, 457-466 (2015); Zhou and Lu, Nat Rev Cancer 16, 463-478 (2016)). These results suggest that Pin1 inhibition might target αSMA+CAFs (myCAFs) and PDGFRα+CAFs (iCAFs), with the latter contributing to desmoplastic immune suppressive TME by secreting collagens and cytokines (Garg et al., Gastroenterology 155, 880-891 e888 (2018); Hosein et al., Nat Rev Gastroenterol Hepatol 17, 487-505 (2020)).

In some embodiments, treatment with a Pin1 inhibitor (e.g., Pin1i−1, Pin1i−2) results in converting a desmoplastic and immunosuppressive tumor microenvironment to a less desmoplastic and less immunosuppressive tumor microenvironment, e.g., 5% less, 10% less or more. In some embodiments, treatment with a Pin1 inhibitor (e.g., Pin1i−1, Pin1i−2) results in converting a desmoplastic and immunosuppressive tumor microenvironment to a less desmoplastic and more immune responsive tumor microenvironment, e.g., 5% less, 10% less, or more. In some embodiments, treatment with a Pin1 inhibitor (e.g., Pin1i−1, Pin1i−2) results in sensitizing a tumor to chemotherapeutics. In some embodiments, the chemotherapeutic is GEM. In some embodiments, the chemotherapeutic is 5-FU. In some embodiments, treatment with a Pin1 inhibitor (e.g., Pin1i−1, Pin1i−2) results in sensitizing a tumor to immunotherapeutics. In some embodiments, the immunotherapeutic is anti-PD-1. In some embodiments, treatment with a Pin1 inhibitor (e.g., Pin1i−1, Pin1i−2) results in the reduction in the proliferation of cancer-associated fibroblasts, e.g., a 5% reduction, a 10% reduction, or more. In some embodiments, treatment with a Pin1 inhibitor (e.g., Pin1i−1, Pin1i−2) results in the reduction of fibrosis in the tumor microenvironment, e.g., a 5% reduction, a 10% reduction, or more.

It's been demonstrated that Pin1 binds to the pSer929-Pro motif in HIP1R and promotes the HIP1R-actin interaction and HIP1R-mediated endocytosis and lysosomal degradation of PD-L1 and ENT1 in PDAC cells in vitro and in mice as well as human tissues and organoids. Moreover, Pin1 inhibition highly synergizes with αPD1 to promote activated lymphocytes induced apoptosis of human organoids and to dramatically reduce tumor growth and increase overall survival of GDA mice.

In some embodiments, Pin1 inhibition synergizes with αPD1 to promote activated lymphocytes induced apoptosis of human organoids. In some embodiments, Pin1 inhibition synergizes with αPD1 to dramatically reduce tumor growth. In some embodiments, Pin1 inhibition synergizes with αPD1 to increase survival of GDA mice.

Notably, the results herein also indicate that combination treatment with Pin1 inhibitor and αPD1 induces only a small minority of tumors (12.5%) to complete regression, but leads to complete regressions in the vast majority (87.5%) of tumors when given in combination with low-dose GEM in GDA mice, underscoring the importance of cytotoxic damage to cancer cells.

In some embodiments, the combination of one or more Pin1 inhibitors and anti-PD-1 induces complete tumor regression. In some embodiments, the % of tumor regression is between about 10% and about 20%. In some embodiments, the % of tumor regression is about 12.5%. In some embodiments, the combination of one or more Pin1 inhibitors, GEM and anti-PD-1 induces complete tumor regression. In some embodiments, the % of tumor regression is between about 80% and about 90%. In some embodiments, the % of tumor regression is about 87.5%.

The results herein suggest a potential new treatment strategy using Pin1 inhibitors in combination with αPD1 and GEM to render aggressive PDAC eradicable. The experimental design with a Pin1 inhibitor treatment for 3 days before addition of the combination of GEM and αPD1 treatment is consistent with the ability of Pin1 inhibitors to reduce multiple cancer pathways in cancer cells and CAFs, to induce the cell surface expression of PD-L1 and ENT in cancer cells, to synergize with GEM and αPD1 to induce organoid apoptosis, and to have better efficacy in PDOX mice. It may be important, prior to GEM and αPD1 treatments, to ‘prime’ the tumor with Pin1 inhibition to reduce multiple cancer pathways in cancer cells and CAFs, and induce the cell surface expression of PD-L1 and ENT in cancer cells to prepare the tumor and its microenvironment for immunochemotherapy.

In some embodiments, the one or more Pin1 inhibitors is introduced prior to the introduction of GEM and αPD1. In some embodiments, the prior introduction may result in priming the tumor to reduce multiple cancer pathways in cancer cells and CAFs and/or in inducing the cell surface expression of PD-L1 and ENT in cancer cells to prepare the tumor and its microenvironment for immunochemotherapy.

The data herein shows that the covalent Pin1 inhibitor sulfopin, which targets the ATO-binding site (Dubiella et al., Nature Chem Biol, in press (2021)), matches ATRA+ATO with its efficacy in PDAC. The studies herein present pre-clinical data that justify further development of Pin1 inhibitors in preparation for first-in-human trials. There exists potential that PD-L1 expression induced by Pin1 inhibition might aid immune evasion of cancer cells, and hence combination of a Pin1 inhibitor with a ICB might be preferable to capitalize on increased PD-L1 expression to increase the synergy.

In summary, herein is uncovered a unique therapeutic strategy and elucidation of the underlying mechanisms by which targeting Pin1 renders aggressive PDAC eradicable by synergizing with immunochemotherapy.

Compounds of the present invention may be in the form of a free acid or free base, or a pharmaceutically acceptable salt. As used herein, the term “pharmaceutically acceptable” refers to a material, such as a carrier or diluent, which does not abrogate the biological activity or properties of the compound, and is relatively non-toxic, i.e., the material may be administered to a subject without causing undesirable biological effects (such as dizziness or gastric upset) or interacting in a deleterious manner with any of the components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

In some embodiments, the compound of the present application is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. 2H, may afford certain therapeutic advantages resulting from greater metabolic stability, for example, increased in vivo half-life or reduced dosage requirements, and thus may be advantageous in some circumstances.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present application may be made and used in the form of individual isomers and substantially free of other isomers, or in the form of a mixture of various isomers, e.g., racemic mixtures of stereoisomers.

In addition, the compounds of the present invention embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

In some embodiments, the Pin1 inhibitor comprises ATO. In some embodiments, the Pin1 inhibitor comprises ATRA. In some embodiments, the Pin1 inhibitor comprises the combination of ATO and ATRA. In some embodiments, the Pin1 inhibitor comprises sulfopin.

### Methods of Synthesis

In another aspect, the present invention is directed to a method for making a compound of the invention, or a pharmaceutically acceptable salt thereof. Broadly, the inventive compounds or pharmaceutically acceptable salts thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared. In some embodiments, the inventive compounds are prepared using chiral HPLC to separate enantiomers from a racemic mixture.

### Pharmaceutical Compositions

Another aspect of the present invention is directed to a pharmaceutical composition that includes a therapeutically effective amount of a compound of the invention or a pharmaceutically acceptable salt thereof, and a pharmaceutically acceptable carrier. The term “pharmaceutically acceptable carrier,” as known in the art, refers to a pharmaceutically acceptable material, composition, or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, compounds of the invention maybe formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compositions are formulated for oral or intravenous administration (e.g., systemic intravenous injection).

Accordingly, compounds of the present invention may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate, or extended release.

For example, compounds of the invention may be formulated into slow-release formulations. Preferably, slow-release formulations maintain consistent or constant blood levels of compounds of the invention. For example, compounds of the invention may be formulated into slow-release ATRA pellets, which maintain consistent or constant blood levels of ATRA and compounds of the invention. Typically, ATRA has a relatively short half-life in humans, i.e., ˜45 minutes, and can be less effective for treating solid tumors. As described herein, slow-release ATRA pellets maintain constant blood ATRA levels and work well as a PIN-1 inhibitor, particularly in combination with ATO.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of the present invention may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups, and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include excipients such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished using a liquid suspension or crystalline or amorphous material with poor water solubility. Prolonged absorption of the compound from a parenterally administered formulation may also be accomplished by suspending the compound in an oily vehicle.

In certain embodiments, compounds of the invention may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microcapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The inventive compounds may be formulated for buccal or sublingual administration, examples of which include tablets, lozenges, and gels.

The compounds may be formulated for administration by inhalation. Various forms suitable for administration by inhalation include aerosols, mists, or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of the invention may be formulated for topical administration which as used herein, refers to administration intradermally by application of the formulation to the epidermis. These types of compositions are typically in the form of ointments, pastes, creams, lotions, gels, solutions, and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents can transport a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerine, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound or compounds of the invention or a pharmaceutically acceptable salt or a stereoisomer thereof, or a composition including the compound or compounds of the invention or a pharmaceutically acceptable salt or a stereoisomer thereof, effective in producing the desired therapeutic response in a particular patient suffering from a Pin1-mediated disease or disorder. The term “therapeutically effective amount” includes the amount of the compound of the application or a pharmaceutically acceptable salt or a stereoisomer thereof, when administered, may induce a positive modification in the disease or disorder to be treated (e.g., remission), or is sufficient to prevent development or progression of the disease or disorder, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed and the particular pharmaceutically acceptable carrier utilized.

The total daily dosage of the compounds and usage thereof may be decided in accordance with standard medical practice, e.g., by the attending physician using sound medical judgment. The specific therapeutically effective dose for any particular subject will depend upon a variety of factors including the disease or disorder being treated and the severity thereof (e.g., its present status); the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, 10th Edition, A. Gilman, J. Hardman and L. Limbird, eds., McGraw-Hill Press, 155-173, 2001).

Compounds of the present invention and their pharmaceutically acceptable salts may be effective over a wide dosage range. In some embodiments, the total daily dosage (e.g., for adult humans) may range from about 0.001 to about 1000 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosage may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day.

In some embodiments, the human equivalent dose for sulfopin is about 3.2 mg/kg. In some embodiments, the human equivalent dose is about 190 mg for a 60 kg person person (fda.gov/media/72309/download).

### Methods of Use

In some aspects, the present invention is directed to methods of treating diseases or disorders involving dysfunctional (e.g., dysregulated) Pin1 activity, that entails administration of a therapeutically effective amount of a compound or compounds of the invention or a pharmaceutically acceptable salt thereof, to a subject in need thereof.

The diseases or disorders may be said to be characterized or mediated by dysregulated or dysfunctional Pin1 activity (e.g., elevated levels of Pin1 relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health. In some embodiments, compounds of the application may be useful in the treatment of proliferative diseases and disorders (e.g., cancer or benign neoplasms). As used herein, the term “cell proliferative disease or disorder” refers to the conditions characterized by unregulated or abnormal cell growth, or both. Cell proliferative disorders include noncancerous conditions, precancerous conditions, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

In general, methods of using the compounds of the present invention include administering to a subject in need thereof a therapeutically effective amount of a compound of the present invention. In some embodiments, the methods include co-administering a therapeutically effective amound of one or more Pin1 inhibitors, or a pharmaceutically acceptable salt or salts thereof, and a therapeutically effective amound of an additional immunotherapy and/or chemotherapy.

In some embodiments, the methods are directed to treating subjects having cancer. Broadly, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) including leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., brain stem glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, acute promyelocytic leukemia, chronic lymphocytic leukemia, chronic myelogenous leukemia, chronic myeloproliferative disorders, anal cancer, colorectal cancer (e.g., colon cancer, rectal cancer), cutaneous T-cell lymphoma, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, gestational trophoblastic tumor glioma, head and neck cancer, hepatocellular (liver) cancer, Hodgkin's lymphoma, non-Hodgkin's lymphoma, hypopharyngeal cancer, intraocular melanoma, ocular cancer, islet cell tumors (endocrine pancreas), renal cancer (e.g., Wilm's Tumor, clear cell renal cell carcinoma), laryngeal cancer, acute lymphoblastic leukemia, acute myeloid leukemia, hairy cell leukemia, lip and oral cavity cancer, liver cancer, lung cancer (e.g., non-small cell lung cancer and small cell lung cancer), primary central nervous system lymphoma, Waldenstrom's macroglobulinemia, melanoma, intraocular (eye) melanoma, merkel cell carcinoma, mesothelioma malignant, mesothelioma, metastatic squamous neck cancer, multiple endocrine neoplasia syndrome, mycosis fungoids, myelodysplastic syndromes, myelodyplastic/myeloproliferative diseases, multiple myeloma, chromic myeproliferative disorders, nasopharyngeal cancer, neuroblastoma, oral cancer (e.g., mouth cancer, lip cancer, oral cavity cancer, tongue cancer, oropharyngeal cancer, throat cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), merkel cell skin carcinoma, squamous cell carcinoma, supratentorial primitive neuroectodermal tumors, testicular cancer, thymoma, thymoma and thymic carcinoma, thyroid cancer, transitional cell cancer of the renal pelvis and ureter and other urinary organs, urethral cancer, gestational trophoblastic tumor, vaginal cancer and vulvar cancer. In some embodiments, the cancer is triple-negative breast cancer or MYCN-driven neuroblastoma.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver (hepatocellular), brain, lung, colorectal (e.g., colon), pancreas, prostate, ovary, breast, or skin (e.g., melanoma).

As used herein, “cell proliferative diseases or disorders of the hematologic system” include lymphoma, leukemia, myeloid neoplasms, mast cell neoplasms, myelodysplasia, benign monoclonal gammopathy, lymphomatoid papulosis, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), acute myeloid leukemia (AML), acute promyelocytic leukemia (APL), mantle cell lymphoma (MCL) and ALK+anaplastic large cell lymphoma) (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, refractory B-cell non-Hodgkin's lymphoma, and relapsed B-cell non-Hodgkin's lymphoma), childhood lymphomas, and lymphomas of lymphocytic and cutaneous origin, e.g., small lymphocytic lymphoma, primary CNS lymphoma (PCNSL), marginal zone lymphoma (MZL), leukemia, including chronic lymphocytic leukemia (CLL), childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver (hepatocellular)” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the brain may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain.

Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, a precancer or precancerous condition of the lung, benign growths or lesions of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer includes lung neoplasms having histologic and ultrastructural heterogeneity (e.g., mixed cell types).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer. Colon cancer includes malignant colon neoplasms, carcinoma in situ, typical carcinoid tumors, and atypical carcinoid tumors. Colon cancer includes adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon can be characterized by hyperplasia, metaplasia, and dysplasia of the colon.

As used herein, “cell proliferative diseases or disorders of the pancreas” include all forms of cell proliferative disorders affecting pancreatic cells. Cell proliferative disorders of the pancreas may include pancreatic cancer, an precancer or precancerous condition of the pancreas, hyperplasia of the pancreas, and dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the prostate.

The compounds of the present invention may be administered to a patient, e.g., a cancer patient, as a monotherapy or by way of combination therapy, and as a front-line therapy or a follow-on therapy for patients who are unresponsive to front line therapy. Therapy may be “first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received another therapy, such as chemotherapy, radioimmunotherapy, surgical therapy, immunotherapy, radiation therapy, targeted therapy, or any combination thereof. In some embodiments, the immunotherapy is a checkpoint inhibitor (e.g., anti-PD-1, anti-PD-L1), a cell-cycle inhibitor (e.g., palbociclib, ribociclib, abemaciclib), or a targeted therapy (e.g., kinase inhibitor).

The methods of the present invention may entail administration of compounds of the invention or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5 or 6 weeks, and in other embodiments entails a 28-day cycle which includes daily administration for 3 weeks (21 days).

In some embodiments is provided a method of inducing quiescence in CAFs, wherein the CAF is treated with one or more Pin1 inhibitors. In some embodiments is provided a method of inhibiting cytokine production in CAFs, wherein the CAF is treated with one or more Pin1 inhibitors. In some embodiments is provided a method of treating diseases involving dysregulated Pin1 expression, wherein the subject is treated with a combination of one or more Pin1 inhibitors, and a chemotherapeutic and/or immunotherapeutic. In some embodiments is provided a method of treating desmoplastic cancers, wherein the subject is treated with a combination of one or more Pin1 inhibitors, and a chemotherapeutic and/or immunotherapeutic. In some embodiments is provided a method of treating a disease characterized by desmoplastic and/or immunosuppressive tumor microenvironment, wherein the subject is treated with a combination of one or more Pin1 inhibitors, and a chemotherapeutic and/or immunotherapeutic. In some embodiments is provided a method of treating cancer or a proliferation disease, wherein the subject is treated with a combination of one or more Pin1 inhibitors, and a chemotherapeutic and/or immunotherapeutic. In some embodiments, the cancer is PDAC. In some embodiments, the cancer is breast cancer. In some embodiments, the cancer is colorectal cancer. In some embodiments, the cancer is APL. In some embodiments is provided a method of reducing or preventing metastasis/es, wherein the subject is treated with a combination of one or more Pin1 inhibitors, and a chemotherapeutic and/or immunotherapeutic. In some embodiments the metastasis is liver metastasis. In some embodiments, the method comprises administering a Pin1 inhibitor for a first time period, followed by administering a combination of the Pin1 inhibitor with a chemotherapeutic and/or an immunotherapeutic for a second time period. In some embodiments is provided a method of reducing proliferation of CAFs. In some embodiments is provided a method of sensitizing a cancer characterized by desmoplastic and/or immunosuppressive tumor microenvironment to a chemotherapeutic and/or immunotherapeutic. In some embodiments is provided a method of reducing fibrosis in the TME.

### Combination Therapy

Pin1 inhibitors, such as Pin1i−1 or Pin1i−2 may be used in combination with at least one other active agent, e.g., anti-cancer agent or regimen, in treating diseases and disorders. The term “in combination” in this context means that the agents are co-administered, which includes substantially contemporaneous administration, by the same or separate dosage forms, or sequentially, e.g., as part of the same treatment regimen or by way of successive treatment regimens. Thus, if given sequentially, at the onset of administration of the second compound, the first of the two compounds is, in some cases, still detectable at effective concentrations at the site of treatment. The sequence and time interval may be determined such that they can act together (e.g., synergistically to provide an increased benefit than if they were administered otherwise). For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, is provided the combination of Pin1i−1 plus gemcitabine. In some embodiments, is provided the combination of Pin1i−2 plus gemcitabine. In some embodiments, is provided the combination of Pin1i−1 plus anti-PD-1. In some embodiments, is provided the combination of Pin1i−2 plus anti-PD-1. In some embodiments, is provided the combination of Pin1i−1 plus gemcitabine plus anti-PD-1. In some embodiments, is provided the combination of Pin1i−2 plus gemcitabine plus anti-PD-1. In some embodiments, is provided the combination of Pin1i−1 plus 5-FU. In some embodiments, is provided the combination of Pin1i−2 plus 5-FU.

In some embodiments, the combinations are useful for treating cancer, e.g., PDAC, breast cancer, or colorectal cancer. For example, the combinations are useful for treating PDAC or breast cancer. In some embodiments, the cancer is PDAC. In some embodiments, the cancer is breast cancer. In some embodiments, the cancer is characterized by having a desmoplastic and/or immunosuppressive tumor microenvironment.

In some embodiments, provided is a method of treating cancer indicated by a greater therapeutic effect, wherein the subject is treated with a combination of one or more Pin1 inhibitors, and a chemotherapeutic and/or immunotherapeutic. In some embodiments, the greater therapeutic effect is indicated by a significant biomarker(s) level(s) change. In some embodiments, the greater therapeutic effect is indicated by a reduction in tumor size, e.g., a 5%, a 10%, a 25%, a 50%, or a 75% reduction in tumor size. In some embodiments, the greater therapeutic effect is indicated by complete or partial remission of disease, i.e., cancer. In some embodiments, the greater therapeutic effect is indicated by a reduction in the incidence of metastases by, e.g., 5%, 10%, 20%, 30% or more. In some embodiments, the greater therapeutic effect is indicated by preventing metastases. In some embodiments, the greater therapeutic effect is indicated by an improvement in survival time. In some embodiments, the improvement in survival time is relative to treatment with one or more Pin1 inhibitors. In some embodiments, the improvement in survival time is relative to treatment with GEM and anti-PD-1. In some embodiments, the improvement in survival time with combination therapy is relative to treatment with Pin1i−2 alone.

In some embodiments, the treatment regimen may include administration of a compound of the invention in combination with one or more additional therapeutics. The dosage of the additional therapeutic may be the same or even lower than known or recommended doses. See, Hardman et al., eds., Goodman & Gilman's The Pharmacological Basis of Therapeutics, 10th ed., McGraw-Hill, New York, 2001; Physician's Desk Reference 60th ed., 2006. Anti-cancer agents that may be used in combination with the inventive compounds are known in the art. See, e.g., U.S. Pat. No. 9,101,622 (Section 5.2 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy. In some embodiments, the treatment regimen may include immunotherapy. In some embodiments, the immunotherapy is a checkpoint inhibitor (e.g., anti-PD-1, anti-PD-L1), a cell-cycle inhibitor (e.g., palbociclib, ribociclib, abemaciclib), or a targeted therapy (e.g., kinase inhibitor).

In some embodiments, the compound or compounds of the invention and the additional anticancer therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart. The two or more anticancer therapeutics may be administered within the same patient visit.

In some embodiments, the compound or compounds of the invention and the additional agent or therapeutic (e.g., an anti-cancer therapeutic) are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the compound of the present application or a pharmaceutical composition. The kits or pharmaceutical systems of the invention may also include printed instructions for using the compounds and compositions.

These and other aspects of the present application will be further appreciated upon consideration of the following Examples, which are intended to illustrate certain embodiments of the application but are not intended to limit its scope, as defined by the claims.

### EXAMPLES

Broadly, the inventive compounds or pharmaceutically acceptable salts thereof, may be purchased and/or prepared by any process known to be applicable to the preparation of chemically related compounds, including, but not limited to, separation using chiral HPLC.

| Abbreviation | Description |
|---|---|
| ATO | arsenic trioxide |
| ATRA | all-trans retinoic acid |
| TME | tumor microenvironment |
| PDAC | Pancreatic ductal adenocarcinoma |
| PDTX | Patient-derived tumor orthotopic xenograft |
| PDOX | Patient-derived orthotopic xenograft |
| GEM | Gemcitabine |
| G + P | Gemcitabine + anti-PD-1 |
| ICB | Immune checkpoint blockade |
| αPD1 | Anti-PD-1 |
| CAF | Cancer-associated fibroblast |
| IHC | immunohistochemistry |
| EMT | epithelial mesenchymal transition |

### Example 1: Materials and Methods

Further experimental details may be found in Koikawa, K., et al., Cell, 184 (18), 4753-71 (2021), which is incorporated by reference in its entirety.

Key Resources Table

| | | |
|---|---|---|
| Reagent or resource | Source | Identifier |

Antibodies

| | | |
|---|---|---|
| Mouse monoclonal anti- | Bao et al., 2004 | N/A |
| Pin1 | | |
| Rabbit polyclonal anti-Pin1 | Ayala et al., 2003, | N/A |
| | Wulf et al., 2001 | |
| Mouse monoclonal anti- | Santa Cruz | Cat# sc-46660; RRID: AB_628132 |
| Pin1 (clone G-8) | | |
| Mouse monoclonal Alexa | Santa Cruz | Cat# sc-46660 AF647; RRID: AB_628132 |
| Fluor 647 anti-Pin1 (clone | | |
| G-8) | | |
| Rabbit polyclonal anti-Pin1 | Cell Signaling | Cat# 3722; RRID: AB_10692654 |
| | Technology | |
| Mouse monoclonal anti- | Abcam | Cat# ab17147; RRID: AB_443686 |
| CD8 alpha | | |
| Rabbit monoclonal anti- | Abcam | Cat# ab16669; RRID: AB_443425 |
| CD3 (clone SP7) | | |
| Rabbit monoclonal anti- | Abcam | Cat# ab40763; RRID: AB_726545 |
| CD45 (clone EP322Y) | | |
| Rabbit monoclonal anti- | Abcam | Cat# ab182422; RRID: AB_2753196 |
| CD163 | | |
| Rabbit monoclonal anti- | Abcam | Cat# ab52625; RRID: AB_2281020 |
| Cytokeratin 19 | | |
| Rabbit monoclonal anti- | Abcam | Cat# ab207178; RRID: AB_2864720 |
| Fibroblast activation protein | | |
| Rabbit polyclonal anti-Ki67 | Abcam | Cat# ab15580; RRID: AB_443209 |
| Rabbit polyclonal anti- | Abcam | Cat# ab4059; RRID: AB_304251 |
| Granzyme B | | |
| Recombinant Alexa Fluor | Abcam | Cat#ab201527; RRID: AB_2890211 |
| 488 Anti-HLA-DPB1 | | |
| antibody (clone EPR11226) | | |
| Recombinant Alexa Fluor | Abcam | Cat# ab202509; RRID: AB_2868435 |
| 555 Anti-alpha smooth | | |
| muscle Actin antibody | | |
| (clone EPR5368) | | |
| Goat F(ab) Anti-Mouse IgG | Abcam | Cat# ab6668; RRID: AB_955960 |
| H&L | | |
| Rabbit polyclonal anti- | Bethyl | Cat# A301-720A; RRID: AB_1210897 |
| FBW7 | | |
| Rat monoclonal anti-Mouse | BioLegend | Cat# 126408; RRID: AB_1089115 |
| FOXP3, Alexa Fluor 647 | | |
| Rat monoclonal anti-Mouse | BioLegend | Cat# 127610; RRID: AB_1134159 |
| Ly-6G, Alexa Fluor 647 | | |
| Rat monoclonal anti-Mouse | BioLegend | Cat# 117312; RRID: AB_389328 |
| CD11c, Alexa Fluor 647 | | |
| Rat monoclonal anti-Mouse | BioLegend | Cat# 100724; RRID: AB_389326 |
| CD8α, Alexa Fluor 647 | | |
| Rabbit polyclonal anti-Akt | Cell Signaling | Cat# 9272; RRID: AB_329827 |
| | Technology | |
| Rabbit monoclonal anti-c- | Cell Signaling | Cat# 9165; RRID: AB_2130165 |
| Jun (clone 60A8) | Technology | |
| Mouse monoclonal anti-E- | Cell Signaling | Cat# 14472; RRID: AB_2728770 |
| Cadherin (clone 4A2) | Technology | |
| Mouse monoclonal anti-Pan | Cell Signaling | Cat# 4523S; RRID: AB_836889 |
| Keratin (clone C11) | Technology | |
| Rabbit monoclonal anti-NF- | Cell Signaling | Cat# 8242; RRID: AB_10859369 |
| kappaB p65 (clone | Technology | |
| D14E12) | | |
| Rabbit monoclonal anti- | Cell Signaling | Cat# 9091; RRID: AB_2687579 |
| LAMP1 (clone D2D11) | Technology | |
| Rabbit monoclonal anti-PD- | Cell Signaling | Cat# 13684; RRID: AB_2687655 |
| L1 (clone E1L3N) | Technology | |
| Rabbit monoclonal anti- | Cell Signaling | Cat# 39749; RRID: AB_2799160 |
| SQSTM1/p62 (clone | Technology | |
| D1Q5S) | | |
| Rabbit monoclonal anti- | Cell Signaling | Cat# 8637; RRID: AB_11217623 |
| RRM1 (clone D12F12) | Technology | |
| Rabbit monoclonal anti- | Cell Signaling | Cat# 5741; RRID: AB_10695459 |
| Vimentin (clone D21H3) | Technology | |
| Rabbit monoclonal anti-β- | Cell Signaling | Cat# 8480; RRID: AB_11127855 |
| Catenin (clone D10A8) | Technology | |
| Rabbit monoclonal anti- | Cell Signaling | Cat# 9664S; RRID: AB_2070042 |
| Cleaved Caspase3 (clone | Technology | |
| 5A1E) | | |
| Rabbit monoclonal anti- | Cell Signaling | Cat# 13647S; RRID: AB_2732796 |
| STING | Technology | |
| Mouse monoclonal PE anti- | Cell Signaling | Cat# 8724; RRID: AB_10829611 |
| CD44 (clone 156-3C11) | Technology | |
| Rabbit monoclonal Alexa | Cell Signaling | Cat# 8893; RRID: AB_2797679 |
| Fluor 555 anti-PDGF | Technology | |
| Receptor α (D13C6) XP | | |
| Mouse monoclonal anti- | Dako | Cat# M0851; RRID: AB_2223500 |
| Human Smooth Muscle | | |
| Actin (clone 1A4) | | |
| Rat monoclonal Super | eBioscience | Cat# 63-0081-82; RRID: AB_2637163 |
| Bright 600 anti-CD8α | | |
| (clone 53-6.7) | | |
| Mouse monoclonal eFluor | eBioscience | Cat# 50-0008-80; RRID: AB_2574148 |
| 660 anti-CD8α (clone | | |
| AMC908) | | |
| Goat Polyclonal Alexa | R&D Systems | Cat# FAB8165G; RRID: AB_2728839 |
| Fluor 488 anti-CD4 | | |
| Rabbit polyclonal anti-PD- | Novus | Cat# NBP1-76769; RRID: AB_11024101 |
| L1 | | |
| Mouse monoclonal anti- | Santa Cruz | Cat# sc-74409; RRID: AB_1119055 |
| AQP9 (clone G-3) | | |
| Mouse monoclonal anti- | Santa Cruz | Cat# sc-376126; RRID: AB_10988034 |
| Cytokeratin 19 (clone A-3) | | |
| Mouse monoclonal anti- | Santa Cruz | Cat# sc-20044; RRID: AB_627346 |
| cyclin D1 (clone DCS-6) | | |
| Mouse monoclonal anti- | Santa Cruz | Cat# sc-393099; RRID: AB_2864729 |
| dCK (clone H-3) | | |
| Mouse monoclonal FITC | Santa Cruz | Cat#sc-377283FITC; RRID: N/A |
| anti-ENT1 (clone F12) | | |
| Rabbit polyclonal anti- | Sigma-Aldrich | Cat# HPA026980; RRID: AB_10602801 |
| CMTM6 | | |
| Mouse monoclonal anti- | Sigma-Aldrich | Cat# F1804; RRID: AB_262044 |
| FLAG (clone M2) | | |
| Mouse monoclonal anti-β- | Sigma-Aldrich | Cat# A5441; RRID: AB_476744 |
| Actin | | |
| Rabbit polyclonal anti- | Proteintech | Cat# 11337-1-AP; RRID: AB_2190784 |
| ENT1 | | |
| Rabbit polyclonal anti-HLA | Proteintech | Cat# 15240-1-AP; RRID: AB_1557426 |
| class I ABC | | |
| Rabbit polyclonal anti- | Proteintech | Cat# 16814-1-AP; RRID: AB_2117572 |
| HIP1R | | |
| Rat monoclonal PE- | Invitrogen | Cat# 25-0451-82; RRID: AB_2734986 |
| Cyanine7 anti-mouse CD45 | | |
| Mouse monoclonal Pan | Invitrogen | Cat# 53-9003-82; RRID: AB_1834350 |
| Cytokeratin Alexa Fluor | | |
| 488 (clone AE1/AE3) | | |
| Rat monoclonal Pacific blue | BioLegend | Cat# 100214; RRID: AB_493645 |
| anti-mouse CD3 | | |
| Rat monoclonal APC anti- | BioLegend | Cat# 100516; RRID: AB_312719 |
| mouse CD4 | | |
| Rat monoclonal PE anti- | BioLegend | Cat# 553033; RRID: AB_394571 |
| CD8α | | |
| Rat monoclonal PE anti- | BioLegend | Cat# 101208; RRID: AB_312791 |
| mouse/human CD11b | | |
| Armenian Hamster | BioLegend | Cat# ;117308; RRID: AB_313777 |
| monoclonal PE anti-mouse | | |
| CD11c | | |
| Rat monoclonal PE anti- | BioLegend | Cat# 108708; RRID: AB_313395 |
| mouse NK1.1 | | |
| Rat monoclonal APC anti- | BioLegend | Cat# 141708; RRID: AB_10900231 |
| mouse CD206 | | |
| Rat monoclonal FITC anti- | BioLegend | Cat# 123108; RRID: AB_893502 |
| mouse F4/80 | | |
| Rat monoclonal APC anti- | BioLegend | Cat# 135210; RRID: AB_2159183 |
| mouse CD279 (PD-1) | | |
| Rat monoclonal APC anti- | BioLegend | Cat# 124312; RRID: AB_10612741 |
| mouse CD274 (PD-L1) | | |
| Rat monoclonal FITC anti- | BioLegend | Cat#116506; RRID: AB_313733 |
| mouse H-2Kb (MHC- | | |
| ClassI) | | |
| Rat monoclonal FITC anti- | BioLegend | Cat# 107606; RRID: AB_313321 |
| mouse 1-A/1-E (MHC- | | |
| ClassII) | | |
| Rat monoclonal FITC anti- | BioLegend | Cat# 515403; RRID: AB_2114575 |
| human/mouse Granzyme B | | |
| Rat monoclonal APC anti- | BioLegend | Cat# 106310; RRID: AB_2087653 |
| mouse CD152 (CTLA4) | | |
| Mouse monoclonal Alexa | BioLegend | Cat# 304056; RRID: AB_2564155 |
| Fluor 647 anti-human CD45 | | |
| Rat monoclonal FITC anti- | Invitrogen | Cat# 11-5773-82; RRID: AB_465243 |
| FOXP3 | | |
| Rat monoclonal FITC anti- | Invitrogen | Cat# 11-2231-82; RRID: AB_2572484 |
| CD223 (Lag-3) | | |
| Goat polyclonal anti-Mouse | Invitrogen | Cat# A-10680; RRID: AB_2534062 |
| IgG, IgM (H + L), Alexa | | |
| Fluor 488 | | |
| Goat polyclonal anti-Rabbit | Invitrogen | Cat# A-11008; RRID: AB_143165 |
| IgG (H + L), Alexa Fluor 488 | | |
| Goat polyclonal anti-Mouse | Invitrogen | Cat# A-11005; RRID: AB_2534073 |
| IgG (H + L), Alexa Fluor 594 | | |
| Goat polyclonal anti-Rabbit | Invitrogen | Cat# A-11012; RRID: AB_2534079 |
| IgG (H + L), Alexa Fluor 594 | | |
| Goat polyclonal anti-Rat | Invitrogen | Cat# A-11007; RRID: AB_10561522 |
| IgG (H + L), Alexa Fluor 594 | | |
| Goat polyclonal anti-Mouse | Invitrogen | Cat# A-21235; RRID: AB_2535804 |
| IgG (H + L) Cross-Adsorbed | | |
| Secondary Antibody, Alexa | | |
| Fluor 647 | | |
| Goat polyclonal anti-Rabbit | LI-COR | Cat# 926-32211; RRID: AB_621843 |
| IgG IRDye 800CW | | |
| Horse anti-Mouse IgG | Vector | Cat# PI-2000; RRID: AB_2336177 |
| (H + L) | Laboratories | |
| Goat anti-Rabbit IgG (H + L) | Vector | Cat# PI-1000; RRID: AB_2336198 |
| | Laboratories | |

Bacterial and virus strains

| | | |
|---|---|---|
| Pin1 shRNA lentiviral | Kondo et al., 2015 | N/A |
| particles | | |
| CRISPR Pin1 lentiviral | Kozono et al., | N/A |
| particles | 2018 | |
| GFP (CMV Bsd) lentiviral | GeneTarge | Cat# LVP001 |
| particles | | |

Biological samples

| | | |
|---|---|---|
| Tissue specimens of | Kyushu | Ethics board approval #28-189 |
| patients with PDAC | University | |
| | Hospital, | |
| | Fukuoka, Japan | |
| Archival tissue specimens | Brigham and | IRB approval #1627 |
| of patients with PDAC | Women's | |
| | Hospital, Boston, | |
| | MA | |
| PDAC patient derived | Gilles et al., 2018 | N/A |
| xenograft (PDX) tumor | | |

Chemicals, peptides, and recombinant proteins

| | | |
|---|---|---|
| All-trans retinoic acid | Sigma | Cat. #R2625 |
| (ATRA) | | |
| Arsenic trioxide (ATO) | Sigma-Aldrich | Cat# A1010 |
| ATRA-releasing pellet | Innovative | Cat# V-111 |
| | Research of | |
| | America | |
| Placebo pellet | Innovative | Cat# C-111 |
| | Research of | |
| | America | |
| Sulfopin | Dubiella et al., | N/A |
| | 2021 | |
| NMP | Sigma-Aldrich | Cat# 328634 |
| Kolliphor HS 15 (Solutol) | Sigma-Aldrich | Cat# 42966 |
| DMSO | Corning | Cat# 25-950-CQC |
| Gemcitabine | FRESENIUS | NDC 63323-102-13 |
| | KABI | |
| 5-FU | Sigma | Cat# F6627 |
| In VivoMAb anti-PD-1 | BioXcell | Cat# BE0146 |
| In VivoMAb anti-PD-L1 | BioXcell | Cat# BE0285 |
| In VivoMAb anti-CD8a | BioXcell | Cat# BE0117 |
| In VivoMAb anti-Nk1.1 | BioXcell | Cat# BE0036 |
| In VivoMAb anti-CTLA4 | BioXcell | Cat# BE0032 |
| In VivoMAb rat IgG2a | BioXcell | Cat# BE0089 |
| isotype control | | |
| In Vivo ph 7.0 Dilution | BioXcell | Cat# IP0070 |
| Buffer | | |
| Paclitaxel | MedChemExpress | Cat# HY-B0015 |
| PEG 300 | MedChemExpress | Cat# HY-Y0873 |
| Tween 80 | MedChemExpress | Cat# HY-Y1891 |
| Growth Factor reduced | Corning | Cat# 356231 |
| (GFR) Matrigel | | |
| RPMI 1640 without L- | Corning | Cat# 17-105-CV |
| glutamine and phenol red | | |
| AdDMEM/F12 | Invitrogen | Cat# 12634-010 |
| HEPES | Invitrogen | Cat# H4034 |
| GlutaMax | Invitrogen | Cat# 35050-061 |
| Penicillin/Streptomycin | Invitrogen | Cat# 15140122 |
| B27 | Invitrogen | Cat# 17504044 |
| N-acetyl-L-cysteine | Sigma-Aldrich | Cat# 9165 |
| Wnt-3a | R&D Systems | Cat# 5036-WN-010 |
| R-Spondin 1 | Peprotech | Cat# 120-38 |
| Noggin | Invitrogen | Cat# 120-10C |
| EGF | Peprotech | Cat# AF-100-15 |
| FGF | Peprotech | Cat# C100-26 |
| Nicotinamide | Sigma-Aldrich | Cat# N0636 |
| Y-27263 | Sigma-Aldrich | Cat# Y0503 |
| A83-01 | R&D Systems | Cat# 2939/10 |
| Cell Tracker Green | Life Technologies | Cat# C7025 |
| Cell Tracker Red | Life Technologies | Cat# C34552 |
| Matrigel | Corning | Cat# 356231 |
| Collagen | BD Biosciences | Cat# 354249 |
| Collagenase/dispase | Sigma-Aldrich | Cat# 11097113001 |
| TrypLE Express | GIBCO | Cat# 12604-021 |
| Defined K-SFM | GIBCO | Cat# 10744019 |
| Dynabeads Human T- | GIBCO | Cat# 11161D |
| Activator CD3/CD28 for T | | |
| Cell Expansion and | | |
| Activation | | |
| Dynabeads Mouse T- | GIBCO | Cat# 11456D |
| Activator CD3/CD28 for T | | |
| Cell Expansion and | | |
| Activation | | |
| Recombinant IL-2 | Peprotech | Cat# 200-02 |
| Recombinant IL-2 | Peprotech | Cat# 212-12 |
| Green-fluorescent caspase | Invitrogen | Cat# R37111 |
| 3/7 probe reagent | | |
| Image-iT LIVE Red | Invitrogen | Cat# I35101 |
| Caspase-3 and -7 Detection | | |
| Kit, for microscopy | | |
| Cell Staining Buffer | BioLegend | Cat. #420201 |
| Foxp3/Transcription | eBioscience | Cat# 00-5523-00 |
| Factor Staining Buffer Set | | |
| Intracellular Fixation & | eBioscience | Cat# 88-8824-00 |
| Permeabilization Kit | | |
| RIPA Lysis and Extraction | Thermo Scientific | Cat# 89901 |
| Buffer | | |
| NaF | Sigma-Aldrich | Cat# S1504 |
| NA₃VO₄ | Sigma-Aldrich | Cat# S6508 |
| Aprotinin | Sigma-Aldrich | Cat# A1153 |
| Leupeptin | Sigma-Aldrich | Cat# 62070 |
| Pepstatin | Sigma-Aldrich | Cat# P5318 |
| DTT | Fisher Scientific | Cat# BP172-25 |
| AEBSF | Sigma-Aldrich | Cat# A8456 |
| Chymotrypsin | Sigma-Aldrich | Cat# C7268 |
| Tween 20 | VWR LIFE | Cat# M147 |
| | SCIENCE | |
| HEPES | Sigma-Aldrich | Cat# H3375 |
| Triton X-100 | Sigma-Aldrich | Cat# T9284 |
| Cycloheximide | Sigma-Aldrich | Cat# C104450 |
| Glycerol | Fisher Chemical | Cat# G33-4 |
| Protein A Sepahrose beads | GeneScript | Cat# L00210 |
| β-mercaptoethanol | Millipore | Cat# ES-007-E |
| 4,4-difluoro-1,3,5,7,8- | Life Technologies | Cat# D-3922 |
| pentamethyl-4-bora-3a,4a- | | |
| diaza-s-indacene | | |
| Hematoxylin | Fisher Chemical | Cat# CS401-1D |
| Picrosirius Red Staining Kit | Polysciences | Cat# 24901 |
| Blasticidin | InvivoGen | Cat# ant-b1-05 |
| Puromycin | Sigma | Cat#P8833 |
| MG132 | Sigma | Cat#1211877-36-9 |
| NAE inhibitor, MLN4929 | Calbiochem | Cat# 5.05477.000 |
| 3-MA | Sigma | Cat# M9281 |
| Bafilomycin A1 | Sigma | Cat# 19-148 |
| Chloroquine | CAYMAN | Cat# 14194 |
| | CHEMICAL | |
| | COMPANY | |
| Easy Sep Mouse CD8+ T | STEM CELL | Cat# 19853 |
| Cell Isolation Kit | | |
| EasySep Buffer | STEM CELL | Cat# 20144 |
| Tumor dissociation Kit | Miltenyi Biotec | Cat# 130-960-730 |
| Mouse | | |
| RNeasy Mini Kit | QIAGEN | Cat# 74104 |
| SYBR Green PCR Master | Applied | Cat# 4309155 |
| Mix | Biosystems | |

Critical commercial assays

| | | |
|---|---|---|
| CellTiter-Glo 2.0 Assay | Promega | Cat# G9242 |
| Human IL-6 uncoated | Invitrogen | Cat# 88-7066-88 |
| ELISA Kit | | |
| Human TGF-β uncoated | Invitrogen | Cat# 88-50390-88 |
| ELISA Kit | | |
| Human SDF1α (CXCL12) | Invitrogen | Cat# EHCXCL12A |
| ELISA Kit | | |
| Human IFNα ELISA kit | R&D Systems | Cat# 41100-1 |
| Human cytokine ELISA | Signosis | Cat# EA-4001 |
| plate array I | | |

Experimental models: Cell lines

| | | |
|---|---|---|
| Human: Patient derived | Koikawa et al., | N/A |
| PDAC organoid | 2018a | |
| Human: Patient derived | Koikawa et al., | N/A |
| PDAC cell | 2018a | |
| Human: Patient derived | Endo et al., 2017; | N/A |
| PSC cell | Koikawa et al., | |
| | 2018b | |
| Human: PBMC | Precision for | Lot# 2010113876 |
| | Medicine | |
| Mouse: KPC derived PDAC | Koikawa et al., | N/A |
| organoid | 2018a | |
| Mouse: KPC derived PDAC | Okumura et al., | N/A |
| cell | 2019 | |

Experimental models: Organisms/strains

| | | |
|---|---|---|
| Mouse: C57BL/6 | Jackson | Stock # 000664 IMSR_JAX: 000664 |
| | Laboratories | |
| Mouse: NOD.Cg- | Jackson | Stock # 005557 IMSR_JAX: 005557 |
| prkdc^{scid}ll2rg^{tm1Wjl}/Szj | Laboratories | |
| Mouse: LSL-KRas^{LSLG12D/+} | Jackson | Stock # 008179 IMSR_JAX: 008179 |
| | Laboratories | |
| Mouse: LSL-p53^{R172H/+} | Jackson | Stock # 008652 IMSR_JAX: 008652 |
| | Laboratories | |
| Mouse: Pdx1-Cre | Jackson | Stock # 014647 IMSR_JAX: 014647 |
| | Laboratories | |

Oligonucleotides

| | | |
|---|---|---|
| MISSION shRNA clone for | Sigma-Aldrich | MISSION shRNA clone for human HIP1R |
| human HIP1R | | |
| MISSION shRNA clone for | Sigma-Aldrich | MISSION shRNA clone for human CD274 |
| human CD274 (PD-L1) | | (PD-L1) |
| MISSION shRNA clone for | Sigma-Aldrich | MISSION shRNA clone for human |
| human SLC29A1 (ENT1) | | SLC29A1 (ENT1) |

Software and algorithms

| | | |
|---|---|---|
| BZ-X 800 analyzer software | KEYENCE | BZ-X 800 analyzer software ver. 1.1.1.8 |
| ver. 1.1.1.8 | | |
| Synergyfinder ver. 2 | Ianevski et al., | Synergyfinder ver. 2 |
| | 2020 | |
| CRISPR design tool | ZHANG LAB | CRISPR.mit.edu |
| Guide Design Resources | ZHANG LAB | Zlab.bio/guide-design-resources |
| CytExpert software | Beckman Coulter | Model #B90883 |
| ImageJ | NIH | Imagej.nih.gov/ij |
| Prism 8 | GraphPad | Graphpad.com/scientific-software/prism |
| | Software | |
| BioRender | BioRender.com | App.biorender.com |

### Human PDAC Tissue Samples

Human PDAC tissue samples used in this study came from 167 patients who underwent surgical resections for pancreatic cancer at Kyushu University Hospital (Fukuoka, Japan). The study was approved by the Ethics Committee of Kyushu University and conducted by the Ethical Guidelines for Human Genome/Gene Research enacted by the Japanese Government and Helsinki Declaration.

### Mouse Models of PDAC

The following 4 different commonly used mouse models of human PDAC were used (Bleijs et al., EMBO J 38, e101654 (2019); Day et al., Cell 163, 39-53 (2015)). 1) The KPC (LSL-K-Ras^{LSLG12D/+}; LSL-p53^{R172H/+}; Pdx1-Cre) genetically engineered mouse model (Hingorani et al., Cancer Cell 7, 469-483 (2005). KPC mice were generated by crossbreeding LSL-KrasG12D (B6.129S4-Krastm4Tyj/J Stock No: 008179, a congenic C57BL/6J genetic background, Jackson Laboratories), LSL-p53 (129S-Trp53tm2Tyj/J, Stock No: 008652, a 129S4/SvJae background, Jackson Laboratories, also known As:p53LSL.R172H 129svj), and Pdx1-Cre (B6.FVB-Tg (Pdx1-cre) 6Tuv/J Stock No: 014647, a C57BL/6 genetic background.

Allelic profiling of resulting KPC mice by the B6 panel (Jackson Laboratories) confirmed 129S1/SvImJ with average match of 84.2%, 129S4SvJae with average match of 83.8%, B6N with average match of 65.7% and B6J, with average match of 65.1%. 2) the KPC Genetically engineered mouse-derived orthotopic allografts (GDA) mouse model (Hingorani et al., Cancer Cell 7, 469-483 (2005); Li et al., Comput Struct Biotechnol J 17, 498-506 (2019)). Mouse PDAC cells were established from KPC mouse pancreatic tumor tissues, followed by orthotopically injecting 1×10⁶ mouse PDAC cells in 50 μL Matrigel (356231, Coaning) into the pancreas of female 8 week-old syngeneic immunocompetent C57BL/6 mice (Jackson Laboratories). 3) PDAC Patient-derived Tumor orthotopic xenografted (PDTX) mouse model (Gilles et al., Clin Cancer Res 24, 1734-1747 (2018); Rubio-Viqueira et al., Clin Cancer Res 12, 4652-4661 (2006)). PDAC PDX tumors were obtained from Dr. Muthuswamy and Dr. Hidalgo and divided into 4×2 mm pieces, followed by xenografting orthotopically into the pancreas of female 8-week-old immunodeficient NOD.Cg-Prkdc^{scid} Il12rg^{tm1Wjl}/SzJ (NOD scid gamma; NSG) mice (Jackson Laboratories). 4) PDAC Patient-derived Organoid and CAFs orthotopic xenograft (PDOX) mouse model (Dantes et al., JCI Insight 5, 137809 (2020); (Koikawa et al., Cancer letters 425, 65-77 (2018)). 5×10⁴ patient derived PDAC organoids and 5×10⁴ patient derived CAFs in 50 μL of Matrigel were injected orthotopically into the pancreas of female 8 week-old immunodeficient NSG mice (Jackson Laboratories).

### Human and Mouse PDAC Organoids

Primary human PDAC organoids (PDAC1 and PDAC2 organoid) were established form fresh surgically resected human PDAC tissues from two different patients in Kyushu University, and mouse PDAC organoids were established from KPC mice pancreatic tumor as previously described (Koikawa et al., Cancer letters 425, 65-77 (2018); Koikawa et al., Cancer Lett. 412, 143-154 (2018)); Okumura et al., Int. J. Cancer 144, 1401-1413 (2019)). These organoids were 3D cultured in Growth Factor reduced (GFR) Matrigel (356231, Corning) with complete organoid media, containing AdDMEM/F12 (12634-010, In-vitrogen, CA, USA), medium supplemented with 1M HEPES (Invitrogen), GlutaMax (35050-061, Invitrogen), penicillin/streptomycin (15140122, Invitrogen), B27 (17504044, Invitrogen), N-acetyl-L-cysteine (9165, Sigma-Aldrich Co.), Wnt-3a (5036-WN-010, R&D Systems), R-Spondin 1 (120-38, Peprotech), Noggin (120-10C, Invitrogen), epidermal growth factor (EGF, AF-100-15, Peprotech), fibroblast growth factor (FGF, C100-26, Peprotech), Nicotinamide (N0636, Sigma-Aldrich Co.), Y-27263 (Y0503, Sigma-Aldrich Co.) and A83-01 (2939/10, R&D Systems). To distinguish and visualize PDAC organoids and CAFs in live-cell imaging fluorescence microscopy, PDAC organoids were labeled with green fluorescent protein (GFP) or Cell Tracker™ Green (C7025, Life Technologies).

### Human CAFs and Human/Mouse PDAC Cells

The human CAFs (CAF1 and CAF2) were established in Kyushu University from fresh surgically resected PDAC tissues from two different patients using the outgrowth method, as described previously (Bachem et al., Gastroenterology 156, 907-921 (2005); Koikawa et al., Cancer Lett. 412, 143-154 (2018)). The isolated cells were confirmed to be CAFs by their spindle-shaped morphology, and immunofluorescence staining for aSMA-, vimentin-, CD90-, glial fibrillary acidic protein-, and nestin-positive, and CK19-negative (Endo et al., Gastroenterology 152, 1492-1506 (2017); Koikawa et al., Cancer Lett. 412, 143-154 (2018)), and used within 6 passages. Human PDAC cells (PDAC1 and PDAC2) were isolated form PDAC organoids, which were established surgically resected human PDAC tissues from two different patients in Kyushu University (Koikawa et al., Cancer Lett. 412, 143-154 (2018)), and Mouse PDAC cells were established from pancreatic tumors of KPC mice using the outgrowth method described previously (Okumura et al., Int. J. Cancer 144, 1401-1413 (2019)), and the cells were tested for CK19, SMA, Vimentin and CD45 to verify their identity and purity, and used within 8 passages for all experiments. Cell lines were maintained in Dulbecco's modified Eagle's medium (Sigma-Aldrich Co.) supplemented with 5%-10% fetal bovine serum, streptomycin (100 mg/mL), and penicillin (100 mg/mL) at 37° C. in a humidified atmosphere containing 10% C02. All cell lines were tested negative for mycoplasma contamination. To distinguish and visualize PDAC cells and CAFs in live-cell imaging fluorescence microscopy, PDAC cells were labeled with green fluorescent protein (GFP) or Cell Tracker™ Green (C7025, Life Technologies), whereas CAFs were labeled with Cell Tracker™ Red (C34552, Life Technologies).

### Method Details

### Mouse Experiments

Pin1i−1 (ATRA+ATO) treatment was described as previously (Kozono et al., Nature communications 9, 3069 (2018); Wei et al., Nature Med 21, 457-466 (2015)), so did Pin1i−2 (Sulfopin) treatment (Dubiella et al., Nature Chem Biol, in press (2021)). Briefly, all mice were randomly selected to receive treatments groups. Tumor sizes were measured by weekly palpation with electronic caliper or ultrasound, and when tumors were reached >0.5 cm, the treatment was started. Briefly, animals were placed under general anesthesia with 2%-3% isoflurane once a week. Under general anesthesia, the abdominal wall became very flaccid, and the muscle tone of the mouse was very low. The mouse was scuffed with the left hand to allow for palpation and measurement with the right hand. This allowed to detect the firm pancreatic mass by palpation, and to determine its size by electronic caliper measurements. Mice in our hands did not develop ascites, and the firm upper abdominal mass was easily identified below the rib cage in the left abdomen. In the GDA model and PDX models, palpable tumors predictably were detectable within 4-7 days post implantation. Hence electronic caliper measurements under general anesthesia were our routine method, and confirmatory ultrasound was conducted in some cases (FIG. 8B). In the transgenic KPC mice, where spontaneous tumor development was expected, weekly palpation was started from age 8 weeks. When the tumor by palpation after age 12 weeks could not be detected, the mice were monitored by ultrasound. Once the maximum length >0.5 cm tumor was detected, the mice were randomized to treatment, and then followed tumor growth by electronic caliper measurement. To validate the external caliper measurements, the tumor size was measured prior to euthanization of the mice and then measured the actual tumor size upon necropsy to ascertain concordance. Mice were treated with ATO (2 mg/kg, i.p., 3 times/week, Sigma) and subcutaneous implantation of 5 mg 21-day slow-releasing ATRA pellets (Innovative Research of America) (Pin1i−1) or placebo pellets (Innovative Research of America), or Sulfopin (40 mg/kg, i.p., every day) (Pin1i−2) or vehicle (Sulfopin diluted solution; 5% NMP, 5% Solutol, 20% DMSO), and/or Gemcitabine (10 mg/kg or 20 mg/kg, i.p., weekly) or vehicle (PBS), and/or anti-PD1 (200 μg, i.p., every 3-4 days, BE0146, BioXcell) or vehicle (IgG isotype control, BE0090, BioXcell). To evaluate the effects of CD8+ T cell or NK1.1′ cell depletion in GDA mice, mice were treated with anti-CD8 (200 μg, i.p., twice a week, BE0117, BioXcell) (Pantelidou et al., Cancer Discov. 9, 722-37 (2019)) or anti-NK1.1 (25 μg, i.p., twice a week, BE0036, BioXcell) (Waggoner et al., Nature 481, 394-398 (2011)), or vehicle (IgG isotype control), and/or Sulfopin (40 mg/kg, i.p., every day) (Pin1i−2) or vehicle (Sulfopin diluted solution; 5% NMP, 5% Solutol, 20% DMSO), and/or anti-PD1 (200 μg, i.p., every 3-4 days, BE0146, BioXcell) or vehicle (IgG isotype control). To test Sulfopin and/or PTX and/or anti-CTLA4 combination treatment in GDA mice, mice were treated with Sulfopin (40 mg/kg, i.p., every day) (Pin1i−2) or vehicle (Sulfopin diluted solution; 5% NMP, 5% Solutol, 20% DMSO), and/or PTX (10 mg/kg i.p., weekly) or vehicle (PTX diluted solution; 10% DMSO, 40% PEG300, 5% Tween 80, 45% Saline), and/or anti-CTLA-4 (250 μg, i.p., every 3-4 days, BE0032, BioXcell) or vehicle (IgG isotype control). Tumor volumes were calculated using the formula L×W²×0.52, where L and W represent length and width, respectively (Kozono et al., Nature communications 9, 3069 (2018)). Survival events were scored when mice lost over 10% body weight, tumor burden reached 2.0 cm in diameter or per absolute survival events. All animal experiments were approved by the IACUC of the Beth Israel Deaconess Medical Center, Boston, MA, USA.

### PDAC Organoid and CAF Co-Culture Model

For indirect co-culture model, 5×10³ primary human PDAC organoids were cultured in 50 μL GFR Matrigel (356231, Corning) with Organoid Media and 5×10⁴ primary human CAFs, which were stably transfected with Pin1 shRNA or control vector, were then seeded on the top of Matrigel for 10 days, followed by monitoring organoid growth using Cyntellect Celigo (Cyntellect) and analyzing the organoid size using Cyntellect Celigo software (version 1.3, Cyntellect). Direct co-culture model of PDAC organoids and CAFs was as described (Koikawa et al., 2018a; Koikawa et al., 2018b). Briefly, primary human PDAC organoid cells were transfected with GFP and subjected to organoid culture. Human primary CAFs were pre-treated with Pin1 inhibitor (Pin1i−1 or Pin1i−2), or stably transfected with Pin1 CRISPR KO or vector control. Before the start of co-culture, CAFs were stained in red with cell tracker red (C34452, Invitrogen) for visualization. 1×10⁴ Organoids were co-cultured with 1×10⁵ human primary CAFs on the Matrigel and collagen (354249, BD Biosciences) mixed gel coated 6 well plate, followed by observing time lapse images using BZ-X800 fluorescence microscope (KEYENCE) and examining the organoid area using BZ-X 800 analyzer software (KEYENCE).

### Organoid Apoptosis Assay

For assaying the effects of Pin1 inhibition on GEM sensitivity, 5×10³ primary human PDAC organoids were cultured in 50 μL GFR Matrigel (356231, Corning) for 7 days and then the organoids were treated with control (DMSO) or Pin1 inhibitors (Pin1i−1, or Pin1i−2) for 3 days. The organoids were isolated from Matrigel using collagenase, and seed Matrigel coated 96 well plate, and then treated with control (PBS) or GEM, or control (DMSO) or 5-FU, followed by assaying organoid apoptosis using fluorescent caspase 3/7 and live-cell time lapse imaging for 24 hr. At the start of coculture, a green-fluorescent caspase 3/7 probe reagent (R37111, Invitrogen) and Hoechst (135102, Invitrogen) was added to visualize cells undergoing apoptosis. Apoptotic organoids were monitored by time lapse imaging using BZ-X800 fluorescence microscope (KEYENCE) and quantified using BZ-X800 analyzer (ver. 1.1.1.8, KEYENCE).

For assaying the effects of Pin1 inhibition on anti-PD1 or PDL1 sensitivity, organoids and PBMCs were co-culture as described previously (Dijkstra et al., 2018; Jiao et al., 2017). Human PDAC organoids were treated with control (DMSO) or Pin1 inhibitors (Pin1i−1, or Pin1i−2) for 3 days, and then co-cultured with human PBMCs. Human PBMCs (Precision for Medicine) were stimulated by PDAC organoid culture media, and 8.0×10⁴ PBMCs were incubated with 2 pl CD3/28 beads (11161D, GIBCO) and 30 U recombinant IL-2 (200-02, Peprotech) per well in 96-well plates for 24 hours before starting co-culture. PDAC organoids and activated PBMCs were directly co-cultured at 5:1 ratio on Matrigel (356231, Corning) coated 96 well plate and treated with control (IgG), anti-PD1, or anti-PDL1, or control (PBS+IgG) or GEM+anti-PD1. At the start of co-culture, a green-fluorescent caspase 3/7 probe reagent (R37111, Invitrogen) and Hoechst (135102, Invitrogen) were added to visualize cells undergoing apoptosis. Apoptotic organoids were monitored by live-cell time lapse imaging was started 2 hours after the start of co-culture using BZ-X800 fluorescence microscope (KEYENCE) and quantified using BZ-X800 analyzer (ver. 1.1.1.8, KEYENCE).

For assaying the effects of Pin1 inhibition on GEM and anti-PD1 sensitivity in mouse PDAC organoids, mouse PDAC organoids (KPC organoids) were established from KPC mouse PDAC tumors, and 1×10⁶ KPC organoid cells were orthotopically transplanted into their tumor-free littermates that did not have all the three transgene or female C57BL/6 WT (8 weeks of age, Jackson) mouse pancreas. 4 weeks after transplantation, mouse CD8⁺ T cells were isolated from the KPC tumor-bearing mouse, or tumor-free littermate mouse or C57BL/6 WT mouse spleens using CD8⁺ T cell Isolation Kit (19853, STEM CELL) according to the manufacturer's instructions, and then 8.0×10⁴ CD8⁺ T cells were activated with 2 μl CD3/28 beads (11453D, GIBCO) and 30 U recombinant IL-2 (212-12, Peprotech) per well in 96-well plate for 24 hours before the start of co-culture. At the start of co-culture, a green-fluorescent caspase 3/7 probe reagent (R37111, Invitrogen) and Hoechst (135102, Invitrogen) were added to visualize cells undergoing apoptosis. KPC organoids were treated with control (DMSO) or Pin1 inhibitors (Pin1i−1 or Pin1i−2) for 3 days, and KPC organoids and activated CD8+ T cells were directly co-cultured 5:1 ratio on Matrigel (356231, Corning) coated 96 well plate, and then treated with control (IgG) or anti-PD1, or control (PBS+IgG) or GEM+anti-PD1. Apoptotic organoids were again monitored by time lapse imaging using BZ-X800 fluorescence microscope (KEYENCE) and quantified using BZ-X800 analyzer (ver. 1.1.1.8, KEYENCE).

### Synergy Score Analysis

To analyze the synergistic effects of the combination therapy between Pin1i and chemotherapies (GEM or 5-FU) or checkpoint im-munotherapies (anti-PD-1 or anti-PD-L1), using Organoid apoptosis assay (see Organoid apoptosis assay). Synergy scores were calculated by Synergyfinder ver 2 (synergyfinder.fimm.fi/) (Ianevski et al., Nucleic Acids Res. 48 IW1), W488-W493 (2020)).

### Production of Stable Pin1 KD or KO Cell Lines

Establishment of Pin1 KD and CRISPR KO cells were as descried previously (Kozono et al., Nature communications 9, 3069 (2018); Wei et al., Nature Med 21, 457-466 (2015)). Pin1 guide RNAs (gRNAs) were designed using the online CRISPR design tool (CRISPR.mit.edu/). The gRNA sequences were gRNA-1 AGT-CACGGCGGCCCTCGTCC TGG (SEQ ID NO: 16), gRNA-2 CAGTGGTGGCAAAAACGGGC AGG (SEQ ID NO: 17). The pLentiCRISPR construction was performed according to the protocol provided by the Zhang Lab (zlab.bio/guide-design-resources). Oligos, (F) 5′-CACCC-gRNA and (R) AAAC-gRNA-C, were cloned into the gRNA Cloning Vector (Addgene, plasmid #49536). To obtain single clones of Pin1 KO cells, cells were transfected with the pLenti CRISPR plasmid containing each target gRNA sequence or empty vector, selected with puromycin for 3 days and isolated by colony formation assay or single cell culture. The single clones were validated by immunoblotting analysis and DNA sequencing.

### Construct of HIP1R Point Mutant

Human HIP1R (Huntingtin Interacting Protein 1 Related) (GenBank: NM_003959.3; 3,204 bp ORF sequence) in pcDNA3.1+/C-(K)-DYK was purchased from GenScript (NJ, USA). For constructing the point mutants of the putative Pin1 binding serine929-proline site, Ser929 was substituted by alanine (S929A) using inverse PCR method with primer sets, as described before (Suizu et al., EMBO J. 35, 1346-1363 (2016)). In brief, whole plasmid DNA was amplified by the polymerase chain reaction of 16 cycles with primer sets described below in Universe Hot Start High-Fidelity 2x PCR Master mix (Biotool). After the reaction, template plasmid DNAs (wild-type) were digested by DpnI enzyme (NEB). The amplified mutated linear DNA fragments were self-ligated and circulized in the presence of T4 Polynucleotide kinase (NEB) and T4 DNA ligase (NEB). The mutation site of plasmid DNA was confirmed by sanger DNA sequencing analysis in DF/HCC DNA resource core facility. Protein expression was analyzed by immunoblotting with anti-FLAG (M2) antibody (Sigma). Primer sets for S929A, sense primer; 5′-CCCCCCACCTGAGCCGC-3′ (SEQ ID NO: 18), anti-sense primer5′-; CGTGCTTGTTGGCCTTCACCTTGG-3′ (SEQ ID NO: 19), Primer sets for S1017A, sense primer; 5′-cCCCTGGAGAGGAGGTGGCC-3′(SEQ ID NO: 20), anti-sense primer; 5′-cGCCTGATGCCCCAGCCAG-3′ (SEQ ID NO: 21).

### Production of Stable HIP1R Expressing Cell Lines

To subclone human HIP1R into a lentivirus vector, wild-type or point mutated HIP1R ORF including DYK tag sequence was amplified by the polymerase chain reaction of 20 cycles with primer sets described below in Q5 High-Fidelity DNA Polymerase reaction mix (NEB). The digested PCR fragments with restriction enzymes XhoI and NotI were ligated into lentivirus backbone plasmid vector pCSII-EF1c-MCS-IRES2-Blasticidin (Suizu et al., 2016). For lentivirus production, 293FT packaging cells were transfected with lentiviral plasmid (pCSII), packaging plasmid (pcDNA-AR8.91), and envelope plasmid (VSV-G/pMD2.G) by PEI (Polyscience) trans-fection method. After transfection for 24 hrs, the transfection reagent was replaced by fresh medium. After incubation at 35° C., 5% CO2 for 48 hr, the resulting lentivirus supernatant was collected and filtrated with 0.45 μm disc filter. Patient-derived PDAC cells were infected with the lentivirus supernatant and fresh media at 1:1 ratio with 8 μg/mL polybrene. After infection for 48 hr, the virus particles are replaced by fresh media and the stably HIP1R-expressing cells were selected in the presence of 2 μg/mL of Blasticidin for at least 4 days. Protein expression was analyzed by immunoblotting with anti-FLAG (M2) antibody (Sigma). Primer sets for subcloning Hs HIP1R into pCSII lentivirus vector, sense primer with XhoI enzyme site; 5′-ATCATCCTCGAGCCACCATGAACAG-CATCAAG-3′ (SEQ ID NO: 22), anti-sense primer including DYK with NotI enzyme site; 5′-ATCGCGGCCGCTCACTTATCGTCGTCATCCTTG-TAATCG-3′ (SEQ ID NO: 23).

### Production of Stable HIP1R KD, ENT KD, and PD-L1 KD Cell Lines

For silencing endogenous HIP1, ENT1 or PD-L1 expression, lentivirus producing shRNA targeting human HIP1R, ENT1 or PD-L1 mRNA was utilized. Five individual clones from MISSION® shRNA target set (Sigma) HIP1R (GenBank: NM_003959.1), ENT1 (GenBank: NM_004955.1), PD-L1 (GenBank: NM_014143.2) (Table 3) or pLKO.1 empty vector was co-transfected with a lentivirus packaging and envelope plasmid into 293FT cells as described above. The resulting lentiviral particles were used to infect PDAC by the mix of lentivirus supernatant and fresh media at 1:1 ratio with 8 g/mL polybrene. After infection for 48 hr., the virus particles are replaced by fresh media and the stably shRNA-expressing cells were selected in the presence of 2 μg/mL of Puromycin for at least 4 days. Protein expression was analyzed by immunoblotting with anti-HIP1R (16814-1-AP, Proteintech), ENT1 (11337-1-AP, Protein-tech) or PD-L1 (13684, Cell Signaling Technology) antibody.

### Cell Proliferation Assay

Cells were seeded at a density of 5000 cells (PDAC1 and PDAC2), or 3000 cells (CAF1 and CAF2) per well in 96-well flat-bottomed plates and incubated for 24 h in culture medium. At 24 h, cells were treated with ATO, ATRA, their combination (Pin1i−1), or Sulfopin (Pin1i−2). Control cells received dimethyl sulfoxide (DMSO) at a concentration equal to that of drug-treated cells for 72 hours. The cell viability was determined by CellTiter-Glo® 2.0 Assay (Promega) following the manufacturer's instructions.

### Elisa Assay and Cytokine Array

The concentrations of cytokines in culture media were evaluated using human IL-6 uncoated ELISA (88-7066), human TGF-b uncoated ELISA (88-50390), human SDF-1 (CXCL12) ELISA (EHCXCL12A), and human LIF ELISA (BMS242) from Invitrogen, human IFNa ELISA (41100-1, R&D Systems) and using human cytokine ELISA plate array I (EA-4001, Signosis), according to the manufacturer's instructions.

### Flow Cytometric Analysis

PDAC tumor tissues were finely sliced into 0.5-1.0 mm fragments and dissolved by collagenase/dispase (11097113001, Sigma) for 30 min at 37° C. or by Tumor dissociation Kit (130-960-730, Miltenyi Biotec) according to the manufacturer's instructions. After filtered, cell lysate was collected. To assess cell surface expressions, cells were harvested by non-enzymatic cell dissociation solution, and resuspend in blocking solution (Cell Staining Buffer, BioLegend). Cells were incubated with following antibodies; CD45-Cy7 (250451-82, Invitrogen), CD3-Pacific blue (100214, BioLegend), CD4-APC (100516, BioLegend), CD8a-PE (553033, BioLegend), CD1 1b-PE (101208, BioLegend), CD11c-PE (117308, BioLegend), CD206-APC (141708, BioLegend), F4/80-FITC (123108, Bio-Legend), CD279-APC (PD-1,135210, BioLegend), CD274-APC (PD-L1, 124312, BioLegend), H-2Kb-FITC (MHC-ClassI, 116506, Bio-Legend), 1-A/1-E-FITC (MHC-ClassII, 107606, BioLegend), NK1.1-PE (10870, BioLegend), anti-CD223-FITC (Lag-3, 11-2231-82, Invitrogen) for 60 min on ice. For FOXP3 staining, cells were fixed and permeabilized using Foxp3/Transcription Factor Staining Buffer Set (00-5523, eBioscience), and incubated with FOXP3-FITC (11-5773-82, Invitrogen) for 30 mins at RT. For Granzyme B staining, cells were fixed and permeabilized using Intracellular Fixation & Permeabilization Kit (88-8824-00, eBioscience), and incubated with Granzyme B-FITC (515403, BioLegend) for 30 mins at RT. All antibodies were diluted according to manufacture instruction. Cells were analyzed using CytoFLEX flow cytometer and CytExpert software (Beckman Coulter, IN, USA).

Immunoblotting analysis (IB)

Cultured cells were lysed in RIPA buffer (50 mM Tris-HCl, pH 7.2, 150 mM NaCl, 1% NP40, 0.1% sodium dodecyl sulfate (SDS), 0.25% Na-deoxycholate) with freshly added phosphatase inhibitors containing 5 mM NaF and 0.2 mM sodium orthovanadate (NA3VO4), and proteinase inhibitors containing 2 μg/mL Aprotinin, 2 μg/mL Leupeptin, 2 μg/mL Pepstatin A, 1 mM DTT (dithiothreitol), 0.5 mM 4-(2-aminoethyl)-benzenesulfonyl fluoride (AEBSF), and 20 mM Chymotrypsin, and then mixed with the SDS sample buffer and loaded onto a gel after boiling for 10 minutes at 95° C. The proteins were resolved by polyacrylamide gel electrophoresis and transferred to PVDF membrane. The transferred membrane was washed three times with Tris-buffered saline containing 0.1% Tween 20 (TBST). After blocking with TBST containing 5% milk or 3% bovine serum albumin (BSA) for 1 h room temperature, the membrane was incubated with the appropriate primary antibody (diluted 1:500-1:10000) in 5% milk or 3% BSA-containing TBST at 4° C. overnight. After incubation with the primary antibody, the membrane was washed three times with TBST for a total of 30 min followed by incubation with horseradish peroxidase conjugated goat anti-rabbit or anti-mouse IgG (diluted 1:5000) for 1 h at room temperature. After three extensive washes with TBST for a total of 30 min, the immunoblots were visualized by enhanced chemi-luminescence. Immunoblotting results were quantified using ImageJ (NIH).

### Immunoprecipitation Analysis (IP)

Cells were lysed in IP lysis buffer (50 mM HEPES, pH 7.4, 150 mM NaCl, 1% Triton X-100 and 10% glycerol) or NP-40 IP lysis buffer (10 mM Tris HCl, pH7.5, 100 mM NaCl, 0.5% NP-40) with freshly added phosphatase and protease inhibitors. After centrifugation at 13,000 g for 15 min, the supernatant was pre-cleaned with Protein A Sepahrose beads (L00210, GeneScript) for 60 min at 4° C., one-tenth of the supernatant was stored as input, and the remainder was incubated for 12 h with 2 μg anti-Pin1 or M2 Flag agarose (Sigma) at 4° C. The supernatants were incubated with Protein A Sepahrose beads for 60 min at 4° C., and then washed three times with the aforementioned lysis buffer. After brief centrifugation, immunoprecipitates were collected, suspended in 2 3 SDS sample buffer (100 mM Tris-HCl, pH 6.8, 4% SDS, 5% b-mercaptoethanol, 20% glycerol, and 0.1% bromophenol blue), boiled for 10 min at 95° C., and subjected to immunoblotting analysis.

### Hip1R Protein Stability Assay

HIP1R WT or HIP1R S929A transfected PDAC cells were incubated with 300 μg/mL cycloheximide (C104450, Sigma) under existing culture conditions. Cells were harvested at the indicated time points with RIPA buffer (50 mM Tris-HCl, pH 7.2, 150 mM NaCl, 1% NP40, 0.1% sodium dodecyl sulfate (SDS), 0.25% Na-deoxycholate) and a proteinase and phosphatase inhibitors (5 mM NaF and 0.2 mM sodium orthovanadate (NA3VO4), 2 μg/mL Aprotinin, 2 μg/mL Leupeptin, 2 μg/mL Pepstatin A, 1 mM DTT (dithiothreitol), 0.5 mM 4-(2-aminoethyl)-benzenesulfonyl fluoride (AEBSF), and 20 mM Chymotrypsin). The supernatant was collected and subjected to western blot analysis using antibody for HIP1R.

### Quantitative Real-Time PCR Analysis

The purification of high-quality RNA from cells was performed using The RNeasy Mini Kit (74104, QIAGEN) in accordance with the manufacturer's protocols. Samples were performed in triplicates. SYBR Green PCR Master Mix (4309155, Applied Biosystems) was used for two-step real-time RT-PCR analysis on an Applied Biosystems StepOnePlus Real Time PCR instrument.

Expression value of the targeted gene in a given sample was normalized to the corresponding expression of GAPDH. The 2-^{ΔΔCt} method was used to calculate relative expression of the targeted genes. The primers were: GAPDH-F, 5′-AGCCTCAAGATCATCAG CAATG′ (SEQ ID NO: 24), GAPDH-R 5′-TGATGGCATGGACTGTGGTCAT-3′ (SEQ ID NO: 25), hPin1-F, 5′-GCCTCACAGTTCAGCGACT-3′ (SEQ ID NO: 26), hPin1-R, 5′-ACTCAGTG CGGAGGATGATGT-3′ (SEQ ID NO: 27), hENT1-F, 5′-CAGAAAGTGCCTTCGGCTAC-3′ (SEQ ID NO: 28), hENT1-R, 5′-TGGGCTGAGAGAGTTGGAGACT-3′ (SEQ ID NO: 29), hPD-L1-F, 5′-TGGCATTTGCTGAACGCATTT-3 (SEQ ID NO: 30), hPD-L1-R, 5′-TGCAGCCAGGTCTAATTGTTTT-3′ (SEQ ID NO: 31) (Zhang et al., 2018), hPD-L1-2F, 5′-GGT GCCGACTACAAGCGAAT-3′ (SEQ ID NO: 32), hPD-L1-2R, 5′-AGCCCTCAGCCTGACATGTC-3′ (SEQ ID NO: 33) (Burr et al., 2017), hPD-L1-3F, 5′-ATTTGGAG GATGTGCCAGAG-3′ (SEQ ID NO: 34), hPD-L1-3R, 5′-CCAGCACACTGAGAATCAACA-3′ (SEQ ID NO: 35) (Mezzadra et al., 2017), hPD-L1-4 F, 5′-CCTACTGG CATTTGCTGAACGCAT-3′ (SEQ ID NO: 36), hPD-L1-4 R, 5′-ACCATAGCTGATCATGCAGCGGTA-3′ (SEQ ID NO: 37).

### Lipid Droplet Accumulation Assay

Lipid droplet accumulation assay was performed as described previously (Endo et al., 2017). Cells were stained with 1 mg/mL 4,4-difluoro-1,3,5,7,8-pentamethyl-4-bora-3a,4a-diaza-s-indacene (bodipy 493/503; #D-3922; Life Technologies, Carlsbad, CA), and the number of bodipy-positive punctures per cell in 20 cells was counted.

### Immunohistochemistry Analysis (IHC)

In vitro PDAC organoids or in vivo mouse pancreas tissues were cut into 4-mm-thick sections from paraffin-embedded samples, de-paraffinized in Histoclear, and rehydrated through an ethanol gradient. Antigen retrieval was achieved by microwaving or autoclaving the sections in sodium citrate buffer (pH 6.0) or Tris-EDTA (pH 8.0). Endogenous peroxidase activity was blocked by incubation with 3% hydrogen for 15 min. After blocking with 5% Goat serum for 30 min at room temperature (RT), the sections were incubated with antibodies in 1% Goat serum buffer (1:1000) overnight at 4° C. Then the sections were incubated with HRP-conjugate secondary antibodies (1:1000, Mouse IgG; PI-2000 or Rabbit IgG; PI-1000, VECTOR) for 60 min at RT. Counterstaining was performed with hematoxylin. Sirius red staining was conducted using a Picrosirius Red Staining Kit (24901, Polysciences, Inc.), according to the man-ufacturer's instructions. Whole-tissue slide scans at 4× magnification was performed on BZ-X800 fluorescence microscope (KEYENCE), and scanned at least three different representative areas at 10× magnification. Image analysis was performed by thresh-olding for positive staining and normalizing to total tissue area, using ImageJ (NIH) and BZ-X800 analyzer (KEYENCE). IHC intensity was semi-quantified manually in a double-blind manner as a 3-tier scale (0; negative to weak, 1; moderate, 2; strong) based on previous reports (Kozono et al., 2018; Wei et al., 2015; Yamaki et al., 2017) (Kozono et al., Nature communications 9, 3069 (2018); Mare'chal et al., Gastroenterology 143, 664-674 (2012); Wei et al., Nature Med 21, 457-466 (2015); Yamaki et al., Int. J. Clin. Oncol. 22, 726-733 (2017)). For Pin1 analysis, Low staining was defined as a staining intensity of “0” or “1 in under 50% of cancer or CAF cells,” and high staining was defined as a staining intensity of “2” and “1 in >50% cancer or CAF cells.”

### Immunofluorescence Analysis (IF)

Cells were seeded on cover glass (#12-542A Fisher Scientific) at approximately 60% confluence and subsequently treated with Pin1i−1 or Pin1i−2 for 72 hours. At 72 hours, cells were fixed by 4% PFA for 20 min, and washed three times with PBS, each time for 5 min. Cells were then permeabilized with 0%-0.1% Triton X-100 and blocked in 3% BSA in PBS for 1 hour at RT. Human PDAC or mouse tumor tissue sections were boiled in 10 mM sodium citrate (pH 6.0), for antigen retrieval after deparaffinization. The sections were permeabilized with PBS containing 0.1%-0.5% Triton X-100 and blocked with PBS containing 5% Goat serum for 30 min RT. The primary antibodies were diluted in PBS containing 1% Goat serum (1:100) and incubated in slides for overnight at 4° C. The cells were rinsed by PBS three times, each time for 5 min. Secondary antibodies were diluted in PBS (1:200) and incubated for 20 min at room temperature. 20 μg/mL DAPI was used to label nuclear of cells. Whole-tissue slide scans at 4× magnification was performed on BZ-X800 fluorescence microscope (KEYENCE), and scanned at least three different representative areas at 10× magnification (for tissue analysis) or 20× magnification (for cell analysis). Image analysis was performed by thresholding for positive staining and normalizing to total tissue area, using ImageJ (NIH) and BZ-X800 analyzer (KEYENCE).

t-CyCIF Experimental Protocol

t-CyCIF imaging consisted of multiple cycles of antibody incubation, imaging, and fluorophore inactivation. The t-CyCIF experimental protocol was conducted as previously described (Du et al., Nat. Protoc. 14, 2900-2930 (2019); Lin et al., eLife 7, e31657 (2018)). In brief, 5 micron sections from 9 FFPE pancreatic adenocarcinoma specimens were baked at 60° C. for 30 min, dewaxed using Bond Dewax Solution (Leica Biosystems) at 72° C., and antigen retrieval was performed with Epitope Retrieval 1 Solution (Leica Biosystems) at 100° C. for 20 minutes using the BOND RX Auto-mated IHC/ISH Stainer (Leica Biosystems). All antibodies were diluted in Odyssey Intercept Buffer (plus Hoechst 33342 0.25 μg/mL; LI-COR Biosciences) and incubated overnight at 4° C. in the dark. See the Key Resources Table for the complete list of antibodies (note that PDGFRα staining was non-specific and inadequate to detect iCAFs). Slides were coverslipped using 20%-50% glycerol solution (Sigma-Aldrich) in PBS. Images were taken using DAPI, FITC, Cy3, and Cy5 channels on the RareCyte CyteFinder Instrument (20x/0.75NA objective lens, RareCyte Inc. Seattle WA). After imaging, the fluorophores were inactivated by incubating with photo-bleaching solution (4.5% H₂O₂ and 20 mM NaOH in PBS) for 30 minutes under LED lights (this step was repeated twice).

t-CyCIF image processing

The image processing of tissue cyclic immunofluorescence is organized in the following steps, each of which is described in detail below: i) the software ASHLAR is used to stitch, register, and correct for image acquisition artifacts (using the BaSiC algorithm). The output of ASHLAR is a single pyramid ome.tiff file for each region imaged; ii) the ome.tiff file is re-cut into tiles (typically 5000×5000 pixels) containing only the highest resolution image for all channels. One random cropped image (250×250 pixels) per tile is outputted for segmentation training (using ImageJ/Fiji); iii) using the ilastik software the labeling of nuclear, cytoplasmic and background areas are trained on the cropped images. Based on the user training the Ilastik software outputs a 3-color RGB image with label probabilities; iv) the RBG probability images are thresholded and watershed in MATLAB to segment the nuclear area. The cytoplasmic measurements are derived by dilating the nuclear mask; v) single-cell measurements are extracted for each channel (cell pixel median and mean for both nuclear and cytoplasmic area) as well as morphological measurements of area, solidity, and cell coordinates location.

### BaSiC

The BaSiC ImageJ plugin tool was used to perform background and shading correction of the original images (Peng et al., Nat. Commun. 8, 14836 (2017)). The BaSiC algorithm calculates the flatfield, the change in effective illumination across an image, and the darkfield, which captures the camera offset and thermal noise. The dark field correction image is subtracted from the original image, and the result is divided by the flatfield image correction to obtain the final image.

### ASHLAR

Alignment by Simultaneous Harmonization of Layer/Adjacency Registration (ASHLAR) is used to stitch together image tiles and register image tiles in subsequent layers to those in the first layer (Lin et al., eLife 7, e31657 (2018); Rashid et al., Sci. Data 6, 323 (2019)). For the first image layer, neighboring image tiles are aligned to one another via a phase correlation algorithm that corrects for local state positioning error. A similar method is applied for subsequent layers to align tiles to their corresponding tile in the first layer. ASHLAR outputs an OME-TIFF file containing a multi-channel mosaic of the full image across all imaging cycles. Full codes available at: github.com/labsyspharm/ashlar.

### Ilastik

ilastik is a machine learning based bioimage analysis tool that is used to obtain nuclear and cytoplasmic segmentation masks from OME-TIFF files (Berg et al., Nat. Methods 16, 1226-1232 (2019)). For increased processing speed, randomly selected 250×250 pixel regions from the original OME-TIFF are used as training data. ilastik's interactive user interface allows the user to provide training annotations on the cropped regions. Users are presented with a subset of the channels stacked images and label pixels as either nuclear area, cytoplasmic area, or background area. The annotations are used to train non-linear classifiers that are applied to the entire image to obtain probability masks describing the probabilities of each pixel belonging to the nuclear, cytoplasmic, or background area. A MATLAAB (version 2018a) script uses these masks to construct binary masks for nuclear and cytoplasmic area.

### T-CyCIF Data Analysis Workflow

The data analysis is divided in a set of pre-processing steps in which data from different tissues is i) log 2-transformed and aggregated together, ii) filtered for image analysis errors, and iii) normalized on a channel-by-channel basis across the entire data from a single experiment. All the steps are performed in MATLAB.

### Data Aggregation

The image processing workflow outputs one ome.tiff image and one data file (.mat) for each tissue area imaged. The data matrices from each .mat file are concatenated into a single matrix for each metric measured (median/mean, nuclear/cytoplasmic) into a single structure (“AggrResults”). The morphological data (i.e., area, solidity, and centroid coordinates) is concatenated into a single structure (“MorpResults”), which also contains the indexing vector to keep track of the tissue of origin within the dataset.

### Data Filtering

Single cells are filtered to identify and potentially exclude from subsequent analysis errors in segmentation and cells lost through the rounds of imaging. Two types of criteria are used to filter cells: morphological criteria based on cell object segmented area, which are applied to all the rounds for the cell object, and DAPI-based criteria which are applied to the DAPI measurement for each imaging round. The latter corrects for cell loss during cycling and computational misalignment, which are both round specific.

Morphological filtering criteria are: 1) nuclear area within a user-input range; 2) cytoplasmic area within a user-input range; 3) nuclear object solidity above a user-input threshold. DAPI-based criteria are: 1) nuclear DAPI measurement above a user-input threshold; 2) ratio between nuclear and cytoplasmic DAPI measurement above a user-input threshold. The filter information for the criteria is allocated to a logical (0-1) structure ‘Filter’, which is used to select the cells to analyze in the further analysis by indexing. The threshold selection is dataset dependent and is performed by data inspection. The values used in each dataset are available with the codes used for data analysis in the github repository (github.com/santagatalab/2021_Koikawa_et_al_CyCIF_codes).

### Data Normalization

Each channel distribution is normalized by probability density function (pdf) centering and rescaling. The aim is to center the distribution of the log 2 fluorescent signal at 0 and rescale the width of the distribution to be able to compare across channels. The data is first log-transformed (base 2). The standard normalization is performed using a 2-component Gaussian mixture model, each model capturing the negative and the positive cell population. If the 2-component model fails to approximate the channel distribution, two other strategies are attempted: i) a 3-component model is used assuming the components with the two highest means are the negative and positive distribution (i.e., discarding the lowest component) or ii) the user selects a percentage ‘x’ of assumed positive cells and a single Gaussian distribution fit is performed on the remainder of the data to capture the negative distribution. The single Gaussian fit is then used as the lower component in a 2-component model to estimate the distribution of the positive population. The strategy chosen for each channel in each dataset is available in the github repository (github.com/santagatalab/2021_Koikawa_et_al_CyCIF_codes). The “add_coeff” is defined as the intersection of the negative and positive distributions. The “mult_coeff” is defined as the difference between the mean of the negative and positive distributions. The full distribution is normalized by subtracting the add_coeff and dividing by the mult_coeff. The normalization is performed on the nuclear and cytoplasmic single-cell, single-channel distributions individually. The data preprocessing workflow is performed on all datasets. The individual analyses used in the paper are performed only in select datasets as follows.

### Isolation of CAF Subsets

Cells from tissue-based experiments are classified into lineage compartments by cell type markers, by gating on the sign of the normalized values of cell type markers. Stromal cells were defined as double negatives for pan-cytokeratin and CD45. Stromal cells were subtyped by k-means clustering based on normalized values of alpha smooth muscle actin, CD44 and DPB1.

### Quantification and Statistical Analysis

Biochemical experiments in vitro were routinely repeated at least three times, and the repeat number was increased according to effect size or sample variation. The sample size was estimated considering the variation and mean of the samples. No statistical method was used to predetermine sample size. No animals or samples were excluded from any analysis. Animals were randomly assigned groups for in vivo studies; no formal randomization method was applied when assigning animals for treatment. Group allocation and outcome assessment was not done in a blinded manner, including for animal studies. A computer program Prism 8 (GraphPad Software, CA, USA) was used for statistical analysis. All data are presented as the means±s.d., followed by determining significant differences using the unpaired Student's t test or one-way analysis of variance (ANOVA) test or Pearson's chi-square test. Kaplan-Meier survival analysis was used for all survival studies, and the groups were compared using the log-rank test. Differences of *p<0.05, **p<0.01, ***p=0.001, and ****p<0.001 were considered statistically significant.

### Example 2: Targeting Pin1 Disrupts the Desmoplastic and Immunosuppressive TME and Renders PDAC Tumors Eradicable by Immunochemotherapy in KPC Mouse-Derived Allografts

To evaluate the effects of targeting Pin1 on the TME and tumor growth in PDAC, two different Pin1 inhibitors (Pin1i−1 and Pin1i−2) were used in three different PDAC mouse models (Bleijs et al., EMBO J 38, e101654 (2019); Day et al., Cell 163, 39-53 (2015)). Pin1i−1 is a combination of clinically available ATRA in a slow-releasing formulation+ATO (Kozono et al., Nature communications 9, 3069 (2018); Wei et al., Nature Med 21, 457-466 (2015)), and Pin1i−2 is sulfopin, a highly Pin1-specific covalent inhibitor that targets the ATO-binding pocket and has no detectable side effect (Dubiella et al., Nature Chem Biol, in press (2021)).

Table 1 is a table of blood test results together with body weight of Pin1i−2 treated non-tumor-bearing B6 mice. See also FIG. 1A-FIG. 1G, FIG. 8A-FIG. 8O, and FIG. 9A-9L. WT B6 mice (non-tumor-bearing mice) were treated with vehicle or Pin1i−2 (20, 40, or 60 mg/kg, i.p., daily) for 5 weeks, followed by examining mice body weight or the RBC, Hb, Plate, and kidney and liver function using blood tests at 3 weeks and 5 weeks (n=5).

Table 2 is a table of immune profiling of splenocytes from Pin1i−2 treated non-tumor-bearing B6 mice. See also FIG. 1A-FIG. 1G, FIG. 8A-FIG. 8O, and FIG. 9A-9L. WT B6 mice (non-tumor-bearing mice) were treated with vehicle or Pin1i−2 (40 mg/kg, i.p., daily) for 5 weeks, followed by examining the immune profile of splenocytes using flow cytometry (n=5).

TABLE 1
Blood test and Body Weight Results for Pin1i-2 Treated B6 Mice

| | | 3 weeks | | | | 5 weeks | | | | Normal | |
|---|---|---|---|---|---|---|---|---|---|---|---|
| | | Control | 20 mg/kg | 40 mg/kg | 60 mg/kg | Control | 20 mg/kg | 40 mg/kg | 60 mg/kg | Range | |
| CBC | WBC | 8 | 7.508 | 4.76 | 6.712 | 7.736 | 7.452 | 5.876 | 5.664 | 1.8-10.7 | (K/uL) |
| | NE | 9.962 | 6.5 | 6.97 | 12.606 | 5.874 | 6.134 | 8.926 | 7.656 | 6.6-38.9 | (%) |
| | LY | 81.756 | 84.982 | 84.522 | 77.548 | 83.104 | 84.51 | 81.206 | 81.742 | 55.8-91.6 | (%) |
| | MO | 8.07 | 8.248 | 8.298 | 9.418 | 10.886 | 9.16 | 9.158 | 10.276 | 0-7.5 | (%) |
| | EO | 0.182 | 0.204 | 0.156 | 0.35 | 0.094 | 0.15 | 0.618 | 0.304 | 0-3.9 | (%) |
| | BA | 0.03 | 0.064 | 0.056 | 0.078 | 0.046 | 0.044 | 0.092 | 0.016 | 0-2 | (%) |
| | RBC | 9.808 | 9.424 | 8.698 | 9.858 | 9.74 | 9.228 | 9.714 | 8.958 | 6.36-9.42 | (M/uL) |
| | Hb | 14.06 | 13.8 | 12.44 | 14.76 | 14.02 | 13.38 | 13.38 | 12.74 | 11-15.1 | (g/dL) |
| | PLT | 499.2 | 530.4 | 449.2 | 700.8 | 624.8 | 612.6 | 548.8 | 618 | 592-2972 | (K/uL) |
| Liver | Alb | 3.5 | 3.375 | 3.6 | 3.5 | 3.05 | 2.94 | 2.98 | 2.72 | 1.95-4.74 | (g/dL) |
| | ALT | 43.2 | 41.5 | 41.67 | 50.5 | 48.4 | 41.4 | 46.2 | 49.2 | 30-206 | (U/L) |
| Renal | BUN | 31.4 | 31 | 28.8 | 25.4 | 28 | 30.4 | 26.4 | 22.8 | 17-57.5 | (mg/dL) |
| | Cre | <0.15 | <0.15 | <0.15 | <0.15 | <0.15 | <0.15 | <0.15 | <0.15 | <0.15 | (mg/dL) |

| | | | | | | | | | |
|---|---|---|---|---|---|---|---|---|---|
| Body weight | 109.86639 | 105.5371 | 107.93296 | 104.79592 | 109.35252 | 107.53045 | 106.70391 | 107.34694 | Day 0 = 100(%) |

TABLE 2
Immune Profiling of Splenocytes from Pin1i-2 Treated B6 Mice

| | Vehicle | | Pin1i-2 | | P value |
|---|---|---|---|---|---|
| CD45+CD3+ Cells (% of CD45+ cell) | 32.50 | (4.02 | 27.77 | (3.61) | 0.0861 |
| CD8a+ T-cell (% of CD45+ cell) | 15.74 | (2.12) | 14.30 | (2.60) | 0.3773 |
| PD-1 (MFI) | 103.22 | (32.99) | 67.90 | (15.50) | 0.062 |
| Lag3 (MFI) | 446.16 | (18.39) | 449.84 | (20.48) | 0.7726 |
| CTLA4 (MFI) | 76.74 | (9.28) | 76.08 | (14.79) | 0.9347 |
| MHC-ClassI (MFI) | 9464.20 | (1272.56) | 11456.80 | (778.10) | 0.0174 |
| CD3−CD11b+ Myeloid cells (% of CD45+ cell) | 6.78 | (1.12) | 6.79 | (1.18) | 0.2039 |
| CD11b+Ly6G+ Myeloid Cells (% of CD45+ cell) | 1.33 | (1.98) | 1.71 | (1.78) | 0.4768 |
| PD-L1 (MFI) | 10847.34 | (1050.66) | 12447.46 | (924.87) | 0.0338 |
| F4/80+ Macrophages (% of CD45+ cell) | 3.53 | (0.65) | 3.54 | (2.97) | 0.1995 |
| CD11c+MHC-II+ DCs (% of CD45+ cell) | 1.23 | (0.10) | 1.48 | (0.34) | 0.1438 |
| PD-L1 (MFI) | 91064.82 | (11434.90) | 89012.88 | (5834.26) | 0.73 |

Three mouse models used were patient-derived tumor orthotopic xenograft (PDTX) model in immunodeficient mice (Gilles et al., Clin Cancer Res 24, 1734-1747 (2018); Rubio-Viqueira et al., Clin Cancer Res 12, 4652-4661 (2006)), patient-derived organoid and CAFs orthotopic xenograft (PDOX) model in immunodeficient mice (Koikawa et al., Cancer letters 425, 65-77 (2018)), and the genetically engineered KPC (LSL-K-Ras^{G12D/+}; LSL-p53^{R172H/+}; Pdx1-Cre) mouse-derived orthotopic allograft (GDA) model in syngeneic immunocompetent mice (Hingorani et al., Cancer Cell 7, 469-483 (2005); Li et al., Comput Struct Biotechnol J 17, 498-506 (2019)). Treatment of overt tumor (0.5 cm)-bearing PDTX or GDA mice with Pin1i−1 or Pin1i−2 significantly reduced tumor growth (FIG. 8A-FIG. 8C, and FIG. 9B), collagen deposition as shown by Sirius Red staining (FIG. 1A and FIG. 8D), and CAF activation as shown by immunofluorescence staining (IF) for 2 CAF markers; α-smooth muscle actin (αSMA) or platelet-derived growth factor receptor α (PDGFRα) (FIG. 1B, FIG. 8F, FIG. 8G, and FIG. 8J). Compared with vehicle controls, Pin1i-treated tumors exhibited a more differentiated histology (FIG. 8D and FIG. 8I), as evidenced by reduced vimentin and increased E-cadherin, which are markers for epithelial mesenchymal transition (EMT) (FIG. 8H). Both Pin1 inhibitors also markedly reduced the proliferation of both cancer cells, assayed by Ki67 immunohistochemistry (IHC) (FIG. 8E) and CAFs, assayed by Co-IF for αSMA or PDGFRα and Ki67 (FIG. 1B, FIG. 8F, FIG. 8G, and FIG. 8K). Thus, Pin1 inhibitors suppress tumor growth and progression, and block CAF activation in GDA and PDTX.

To examine whether Pin1 inhibitors affect the immunosuppressive TME, tumor-infiltrating immune cell populations were evaluated by IF and flow cytometry in GDA tumors. Pin1 inhibitors increased CD8α+ T cells, specifically CD8α+ Granzyme B+ cytotoxic T-cells (CTLs) and decreased immunosuppressive CD4+ FOXP3+ regulatory T-cells (Tregs), Ly6G+CD11b+myeloid cells, and F4/80+ CD206+ tumor associated macrophages (TAMs) (FIG. 1C and FIG. 9D-FIG. 9G). Thus, targeting Pin1 disrupts the desmoplastic and immunosuppressive TME in PDAC.

The above results suggest that Pin1 inhibitors might increase PDAC responses to chemo- and/or immunotherapy. To evaluate the effects on chemotherapy, overt tumor-bearing PDTX, PDOX or GDA mice were treated with Pin1i or a low dose (20 mg/kg) of gemcitabine (GEM) or their combination, with or without starting Pin1i treatment 3 days before starting GEM (FIG. 8A). Pin1i−1 and Pin1i−2 were only slightly superior to GEM treatment in inhibiting tumor growth and improving survival, but the effects were significantly enhanced by their combination in each of mouse models assessed (FIG. 8K, FIG. 8L, FIG. 8M, and FIG. 9A). The combination effects were even more profound in a group of PDAC tumor bearing mice with Pin1i treatment started at 3 days before GEM (FIG. 8K), prompting Pin1i treatment routinely starting 3 days before others in combination therapies. In addition, unlike GEM treatment alone, Pin1i also fully prevented liver metastasis of PDAC in PDTX mice (FIG. 8N).

To determine the effects of targeting Pin1 in response to ICB and/or GEM, tumor-bearing GDA mice were treated with Pin1i, anti-PD-1 (PD1), low dose GEM (10 mg/kg)+αPD1 (G+P, immunochemotherapy), Pin1i+αPD1 or Pin1i+G+P (FIG. 8A). Treatment with αPD1 or G+P partially inhibited tumor growth and marginally increased overall survival as reported (Jiang et al., 2016). Both Pin1i−1 and Pin1i−2 as single agents were slightly more potent than above treatments (FIG. 1D-FIG. 1E, FIG. 9B, and FIG. 9I). Notably, Pin1i−1+αPD1 combination dramatically reduced tumor growth and more than doubled median survival, with 12.5% complete regression (FIG. 1E). The most pronounced effect was seen in a group treated with the triple combination. Pin1i−1 and G+P combination markedly disrupted the desmoplastic and immunosuppressive TME (FIG. 9C, FIG. 9F, and FIG. 9G), and fundamentally changed overall survival, leading to 87.5% complete regressions (FIG. 1D and FIG. 1E). These mice survived without macroscopic (FIG. 1F) or microscopic (FIG. 1G) evidence for residual PDAC for at least for 1 year in good health, even though the treatments were stopped at 120 days (FIG. 1E and FIG. 9H). Similar results were obtained with Pin1i−2 in combination with αPD1 or G+P, although less impressive during longterm treatment (FIG. 9B, FIG. 9C, FIG. 9F, FIG. 9H, and FIG. 9I). Of note, the maximum effect of FAK inhibitor+αPD1 is also reported when given with GEM, although without tumor regression (Jiang et al., Nat Med 22, 851-860 (2016)), supporting the notion that GEM remains a cornerstone of PDAC treatment (Amrutkar and Gladhaug, Cancers (Basel) 9, 157 (2017)).

To examine the relevance of cytotoxic and natural killer cells for the synergy between Pin1 inhibition and immunotherapy, the efficacy of Pin1i−2 and αPD1 combination was examined in the absence or presence of CD8α+ T-cells or NK1.1+ cells in GDA mice. Only when CD8α+T-cells were depleted, PDAC tumor growth accelerated considerably and the synergistic efficacy of Pin1i−2 and αPD1 treatment was completely offset (FIG. 1H). αPD1 treatment alone also accelerated tumor growth in CD8α+ T cell depleted GDA mice (FIG. 9J), reminiscent of hyper-progression observed in clinical trials of cancer patients with αPD1 therapy (Wang et al., Mol Cancer 19, 81 (2020)). Immune cell profiling of the tumors by FACS and IF further showed that while Pin1 inhibition increased the number of tumor infiltrating CD8α+ T-cells and their Granzyme B+expression, Pin1i alone also increased their expression of the activation/exhaustion markers, PD-1, lymphocyte activation gene-3 (LAG-3), and cytotoxic T-lymphocyte antigen 4 (CTLA-4), but the later effects tended to be reduced by combination with αPD1 (FIG. 9F and FIG. 9H), indicative of reversion of lymphocyte exhaustion, as shown (Ruscetti et al., Cell 181, 424-441 e421 (2020)). In contrast, PD-L1 expression on myeloid cells and dendritic cells (DCs), which contributes to anti-tumor immune response (Oh et al., Nature Cancer 1, 681-691 (2021)), were not changed after Pin1 inhibition (FIG. 9E). Thus, cytotoxic CD8+ T-cells are pivotal for the synergy between Pin1 inhibition and immunotherapy.

To investigate whether the synergy of immunochemotherapy with Pin1 inhibition in PDAC is specific to gemcitabine or αPD1, the microtubule stabilizing paclitaxel, non-nucleoside-based chemotherapy, and anti-CTLA-4 (αCTLA4), an alternative ICB, were tested. Pin1 inhibition did not potentiate paclitaxel anti-tumor activity and tended to increase αCTLA4 antitumor effect, but the effect was small and without statistical significance (FIG. 9K). Thus, although Pin1 inhibitors have moderate single agent anticancer efficacy, they potently disrupt the desmoplastic and immunosuppressive TME, and remarkably render most aggressive PDAC tumors eradicable when combined with GEM and αPD1 in GDA mice.

### Example 3: Pin1 is Overexpressed Both in Cancer Cells and CAFs in Human PDAC, and Correlates with the Desmoplastic and Immunosuppressive TME, and Poor Patient Survival

Given such unexpected and striking potency of Pin1i in rendering PDAC eradicable, Pin1 expression in human tumor tissues surgically resected from 167 PDAC patients (Endo et al., Gastroenterology 152, 1492-1506 e1424 (2017); Koikawa et al., Cancer letters 412, 143-154 (2018)) and its relationships with TME changes and patient survival using IHC with a validated Pin1 monoclonal antibody was evaluated (Bao et al., Am J Pathol 164, 1727-1737 (2004)). Pin1 was overexpressed in cancer cells and correlated positively with cancer progression (FIG. 2A and FIG. 10A). Furthermore, Pin1 was overexpressed in tumor stromal CAFs, which expressed various CAF markers, αSMA, fibroblast activation protein (FAP), human leukocyte antigen (HLA) class II histocompatibility antigen, DP beta 1 (HLA-DPB1), or CD44 (FIG. 2B-FIG. 2D, and FIG. 10B) (Hosein et al., Nat Rev Gastroenterol Hepatol 17, 487-505 (2020)). By classifying cohorts into Pin1-High and Pin1-Low groups based on their IHC intensity and area, it was not only found that Pin1 was overexpressed in cancer cells in 71.5% of the patients and correlated with poor survival, as reported (Chen et al., Cancer Sci 110, 2442-2455 (2019); Liang et al., Cancer Res 79, 133-145 (2019)), but also that Pin1 was overexpressed in CAFs in 51.9% of the patients and correlated with poor survival (FIG. 2E). More impressively, high Pin1 both in cancer cells and CAFs, as compared with low Pin1 group, strongly correlated with reduced median overall survival from 60 to 16 months in this cohort of surgically resectable PDAC patients (FIG. 2F). To examine whether Pin1 overexpression might be relevant to the desmoplastic and immunosuppressive TME, collagen deposition and tumor-infiltrating immune cell populations in human PDAC tissues were evaluated. Pin1 overexpression in CAFs, but not in cancer cells, significantly correlated with collagen deposition (FIG. 2G). Pin1 overexpression in cancer cells and CAFs also correlated with fewer infiltrated CD8+ T-cells and more CD163+ TAMs (FIG. 2H and FIG. 2I). Thus, Pin1 is overexpressed in cancer cells and CAFs in human PDAC, and strongly correlates with the desmoplastic and immunosuppressive TME, and poor patient survival.

### Example 4: Pin1 Promotes Oncogenic Signaling Pathways, CAF Activation and Crosstalk with Cancer Cells to Enhance Tumor Growth and Malignancy in Human Organoids and PDOXs

To determine the functional significance of Pin1 overexpression in CAFs in human PDAC tumors, primary CAFs were derived from two different human PDAC patients (Koikawa et al., Cancer letters 425, 65-77 (2018); Koikawa et al., Cancer letters 412, 143-154 (2018)) and their Pin1 function was inhibited using Pin1i, genetic knockdown (KD) or CRISPR knockout (KO) (Kozono et al., Nature communications 9, 3069 (2018); Wei et al., Nature Med 21, 457-466 (2015)). Both Pin1i−1 and Pin1i−2 dose-dependently reduced Pin1 and its many substrate oncoproteins in CAFs (FIG. 3A and FIG. 10C), with ATRA and ATO synergistically targeting and degrading Pin1, as shown in other cancer cells (Kozono et al., Nature communications 9, 3069 (2018); Wei et al., Nature Med 21, 457-466 (2015)). Also demonstrated was that Pin1i−1 and Pin1i−2 led to suppression of CAF proliferation (FIG. 3B and FIG. 10D), which was further corroborated in studies using Pin1 KD (FIG. 10E). Pin1i, Pin1 KD or KO also induced quiescent phenotype of CAFs and inhibited their secretion of a wide range of cytokines (FIG. 3C-FIG. 3E, and FIG. 10F). Specifically, suppression of IL-6 and TGF-β release by cancer cells was found as were lower levels of TL-6, TGF-β, LIF and CXCL12 secretion by CAFs in response to Pin1 inhibition (FIG. 10G and FIG. 10H). Those factors are known to promote cancer cell progression (Erkan et al., Gut 61, 172-178 (2012); Shi et al., Nature 569, 131-135 (2019)), prevent T cell recruitment into the TME (Feig et al., Proc Natl Acad Sci USA 110, 20212-20217 (2013); Garg et al., Gastroenterology 155, 880-891 e888 (2018))), induce the desmoplastic and immunosuppressive TME (Mace et al., Gut 67, 320-332 (2018)), and suppress response to anti-PD-L1 (αPD-L1) (Feig et al., Proc Natl Acad Sci USA 110, 20212-20217 (2013); Mariathasan et al., Nature 554, 544-548 (2018)). In summary, Pin1 induces CAF activation and may contribute to the desmoplastic and immunosuppressive TME.

A continuous crosstalk between cancer cells and CAFs enhances tumor growth and invasion. To address whether Pin1 inhibition in CAFs affects their ability to act on cancer cells, 3-dimensional (3D) human primary PDAC organoid indirect co-cultures were established by adding CAFs on the top of established organoids to analyze the effects of CAF-derived humoral factors on cancer cell growth and invasion (FIG. 3F). Unlike control CAFs, which promoted organoid growth and invasion, Pin1 KD primary CAFs failed to promote cancer cell growth and invasion with organoid growth similar to the level of those without adding CAFs (FIG. 3G and FIG. 3H). To visualize cell-cell interactions, green-labeled established PDAC organoids were directly co-cultured with red-labeled Pin1i-treated or KO CAFs, followed by live-cell time-lapse imaging to assay organoid growth and invasion (Koikawa et al., Cancer letters 425, 65-77 (2018)) (FIG. 10I). The results further confirmed that control CAFs, but neither Pin1i nor Pin1-KO CAFs, promoted PDAC organoid growth and invasion (FIG. 10J and FIG. 10K). Finally, to examine whether Pin1 is important for CAFs to promote the TME and tumor growth of PDAC in vivo, the PDOX model was performed by orthotopically co-transplanting human PDAC organoids with Pin1 KO or control human primary CAFs into the mouse pancreas (Koikawa et al., Cancer letters 425, 65-77 (2018)). In contrast to control CAFs, Pin1 KO cells completely failed to promote tumor fibrosis, cancer cell proliferation, and tumor growth and progression, similar to those without co-transplanted CAFs (FIG. 3I, FIG. 3J, FIG. 10L, and FIG. 10M). Thus, Pin1 expression in CAFs is necessary to promote the pro-tumorigenic TME and enhance the growth and malignant phenotype of PDAC in organoids and tumors in PDOX mice.

### Example 5: Pin1 Promotes Oncogenic Signaling Pathways and Reduces the Expression of PD-L1 and ENT1 at the Cell Surface of PDAC Cells

The above results show that targeting Pin1 disrupts the TME and renders PDAC tumors eradicable by immunochemotherapy, but the previous attempts to target stroma cells in PDAC were only modestly effective (Ho et al., Nat Rev Clin Oncol 17, 527-540 (2020); Hosein et al., Nat Rev Gastroenterol Hepatol 17, 487-505 (2020); Neesse et al., Gut (2018); Whittle and Hingorani, Gastroenterology 156, 2085-2096 (2019)). It was reasoned that Pin1 inhibitors might also act on cancer cells, given its overexpression in cancer cells (FIG. 2A-FIG. 2C). Thus, the effects of Pin1 inhibition, Pin1 KD or KO in primary cancer cells and organoids derived from two human PDAC patients (Koikawa et al., Cancer letters 425, 65-77 (2018)) were examined. Indeed, like Pin1 KD or KO, both Pin1 inhibitors dose-dependently reduced Pin1 and its substrate oncoproteins including those in Kras signaling in cancer cells (FIG. 11A-FIG. 11C and FIG. 11E) and reduced their proliferation (FIG. 11D, FIG. 11F, and FIG. 11G), as reported (Chen et al., Cancer Sci 110, 2442-2455 (2019); Liang et al., Cancer Res 79, 133-145 (2019), (Sun et al., PMID: 32347623 (2020)), but the effect was largely abolished by Pin1 KO (FIG. 11H), confirming their specificity (Dubiella et al., Nature Chem Biol, in press (2021); Kozono et al., Nature communications 9, 3069 (2018); Wei et al., Nature Med 21, 457-466 (2015)). Furthermore, like Pin1 KD (FIG. 11G), both Pin1 inhibitors also reduced human PDAC organoid growth and enhanced the ability of GEM to inhibit organoid growth and proliferation (FIG. 111-FIG. 11L). Moreover, when established human PDAC organoids were pre-treated with Pin1i−1 or Pin1i−2 for 72 hrs, followed by treatment with GEM for 24 hrs, both Pin1 inhibitors greatly potentiated GEM-mediated organoid apoptosis, whereas neither Pin1i nor GEM alone had obvious effect (FIG. 4A and FIG. 4B).

Given the striking ability of Pin1 inhibitor pretreatment to potentiate the GEM sensitivity of PDAC cells, it was reasoned that Pin1 inhibitors might affect the expression of some determinants of the GEM sensitivity. Among those, equilibrative nucleoside transporter 1 (ENT1) is required for GEM uptake and is a therapeutic response marker for GEM, deoxycytidine kinase (dCK) is involved in the first phosphorylation cascade in GEM activation, and ribonucleotide reductase subunit 1 (RRM1) is a main nuclear target for GEM (Amrutkar and Gladhaug, Cancers (Basel) 9, 157 (2017)). Both Pin1 inhibitors dose-dependently increased the levels of ENT1, but neither dCK nor RRM1 was affected (FIG. 4C). Since Pin1 inhibition potently enhanced αPD1 efficacy against PDAC (FIG. 1E), it was hypothesized that Pin1 inhibitors might also affect the expression of ICB response biomarkers, whose upregulation increases ICB efficacy such as PD-L1 (Galluzzi et al., Science translational medicine 10, eaat7807 (2018); Jiao et al., Clin Cancer Res 23, 3711-3720 (2017); Zhang et al., Nature 553, 91-95 (2018)) and HLA class 1 (McGranahan et al., Cell 171, 1259-1271 e1211 (2017); Rodig et al., Science translational medicine 10, eaar3342 (2018); Yamamoto et al., Nature 581, 100-105 (2020)). Indeed, both Pin1 inhibitors dose-dependently upregulated PD-L1, but not HLA class 1 level (FIG. 4C). The specificity of PD-L1 and ENT1 upregulation was confirmed by Pin1 KD or KO (FIG. 4D), and was notable on the cell surface of Pin1i-treated PDAC cells in vitro (FIG. 4E and FIG. 4F) and Pin1i-treated PDTXs and GDAs in mice (FIG. 4G-FIG. 4J, FIG. 12A, and FIG. 12B). Moreover, Pin1 expression inversely correlated with that of PD-L1 and ENT1 in human PDAC tissues (FIG. 12C-FIG. 12E). In summary, Pin1 inhibition blocks multiple cancer pathways including those in oncogenic Kras signaling and induces the expression of PD-L1 and ENT1 on the surface of cancer cells in PDAC in vitro and in vivo.

### Example 6: Pin1 Promotes the Endocytosis and Lysosomal Degradation of PD-L1 and ENT1 by Acting on the pS929-Pro Motif in HIP1R

The findings that Pin1 inhibition in PDAC cells increased PD-L1 and ENT1 protein expression levels (FIG. 4C-FIG. 4F) but decreased their mRNA levels (FIG. 12F) suggest that Pin1 inhibition might stabilize ENT1 and PD-L1 proteins. The proteolysis of both PD-L1 (Burr et al., Nature 549, 101-105 (2017); Mezzadra et al., Nature 549, 106-110 (2017); Wang et al., Nat Chem Biol 15, 42-50 (2019); Zhang et al., Nature 553, 91-95 (2018)) and ENT1 (Hu et al., Oncol Rep 38, 2069-2077 (2017)) is highly regulated, and to modulate proteolysis is a dominant mechanism for Pin1 to regulate dozens of oncogenic proteins (Zhou and Lu, Nat Rev Cancer 16, 463-478 (2016)). To assess the mechanisms behind Pin1-mediated effects, PDAC cells were treated with chemical inhibitors for the lysosomal degradation pathway (3-MA, bafilomycin and chloroquine) or proteasomal degradation pathway (MG132 and MLN4924). Inhibition of the proteasomal pathway increased PD-L1, as shown (Zhang et al., Nature 553, 91-95 (2018)), but not ENT1, while inhibition of the lysosomal pathway increased both PD-L1 and ENT1 (FIG. 12G), suggesting that lysosomal degradation may be a common mechanism for Pin1-mediated regulation of PD-L1 and ENT1.

To support this possibility, Huntingtin interacting protein 1-related (HIP1R) (Wang et al., Nat Chem Biol 15, 42-50 (2019)) and CKLF Like MARVEL Transmembrane Domain Containing 6 (CMTM6) (Mezzadra et al., Nature 549, 106-110 (2017)), which promote and inhibit the endocytosis and lysosomal degradation of PD-L1, respectively, and also contain putative Pin1 substrate recognition sites (pSer/Thr-Pro motifs), whereas such Pin1 recognition sites is are not present in PD-L1, were investigated. Both Pin1 inhibitors dose-dependently increased HIP1R with a slower mobility and presumably in phosphorylated form (FIG. 5A and FIG. 5B), which was also induced by Pin1 KD or KO (FIG. 5C and FIG. 5D), but was undetectable after treating cell lysates with calf intestinal phosphatase (CIP) (FIG. 5E). To examine whether Pin1 interacted with phosphorylated HIP1R, co-immunoprecipitation (Co-IP) was performed which demonstrated that Pin1 bound to phosphorylated HIP1R under endogenous conditions, and that this interaction was phosphatase-sensitive (FIG. 5E). By contrast, Pin1 inhibitors neither affected the PD-L1 stabilizing CMTM6, nor did Pin1 interact with CMTM6 (FIG. 5A, FIG. 5C, and FIG. 5E). To determine the importance of HIP1R in regulating PD-L1 and ENT1 levels, HIP1R was stably knocked down in PDAC cells revealing that HIP1R KD increased both PD-L1 and ENT1 levels in these cells (FIG. 5F). These results together suggest that Pin1 promotes PD-L1 and ENT1 degradation by binding to phosphorylated HIP1R.

To identify the Pin1-binding site in HIP1R, an Ala substitution (S929A) at the only putative and conserved S/P site, pSer929-Pro motif in HIP1R, was generated (FIG. 12H). Unlike wild-type (WT) HIP1R, when introduced into PDAC cells, the S929A mutant not only failed to interact with Pin1, but also increased levels of phosphorylated HIP1R as well as PD-L1 and ENT1 (FIG. 5G-FIG. 5I), similar to Pin1 inhibitors, Pin1 KD or KO (FIG. 5A-FIG. 5D). Notably, the pSer929-Pro in HIP1R is located within its actin-binding domain (FIG. 12H), which is critical for HIP1R-mediated endocytosis and lysosomal degradation (Gottfried et al., Biochem Soc Trans 38, 187-191 (2010); Messa et al., eLife 3, e03311 (2014)). These results suggest that the S929A mutation might impair the ability of HIP1R to bind actin and promote the endocytosis of PD-L1 and ENT1 to lysosomes. Indeed, wild type HIP1R, but not its S929A mutant, interacted with actin (FIG. 5J) and colocalized with Pin1, PD-L1 and ENT1 to peri-nuclear lysosomes, as marked by the lysosomal marker LAMP1 (FIG. 5K-FIG. 5N). Cycloheximide (CHX) chase assay demonstrated that the HIP1R, but not its S929A mutant, was unstable (FIG. 12I). Thus, Pin1 promotes the endocytosis and lysosomal degradation of PD-L1 and ENT1 presumably by acting on pSer929-Pro motif in HIP1R to facilitate actin binding.

### Example 7: Targeting Pin1 Synergizes with Immunochemotherapy to Induce PDAC Organoid Apoptosis

Given that targeting Pin1 robustly induces the expression of ENT1 and PD-L1 on the cell surface of PDAC cells, which have been shown to improve the efficacy of GEM (Hu et al., Oncol Rep 38, 2069-2077 (2017); Liu et al., Gastroenterology 158, 679-692 e671 (2020)) and ICB (Deng et al., J Clin Invest 124, 687-695 (2014); Herter-Sprie et al., JCI Insight 1, e87415 (2016); Jiao et al., Clin Cancer Res 23, 3711-3720 (2017); Zhang et al., Nature 553, 91-95 (2018)), respectively, a critical question is whether targeting Pin1 would affect the therapeutic response to GEM and ICB. To address this question, Pin1 was targeted in established PDAC organoids derived from human primary PDAC cells without any stromal cells to avoid indirect effects (Koikawa et al., Cancer letters 425, 65-77 (2018)). As expected, Pin1 inhibitor treatment time-dependently induced ENT1 and PD-L1 levels (FIG. 6A). To assay the effects of Pin1 inhibition on GEM sensitivity, established human PDAC organoids were pre-treated with Pin1i for 3 days, and then with GEM or 5-FU, followed by live-cell time-lapse imaging for 24 hrs to visualize and quantify the dynamic changes in organoid apoptosis (FIG. 6B). Both Pin1 inhibitors time- and dose-dependently sensitized PDAC organoids to GEM-induce apoptosis and the effects were highly synergistic (FIG. 6C-FIG. 6E, and FIG. 13A). The similar effect was observed in Pin1i with 5-FU treatment (FIG. 13G and FIG. 13H). Pin1 KO or introduction of HIP1R929A mutant into the PDAC organoids resulted in similar synergy with GEM treatment (FIG. 12J), further supporting our model. To examine the importance of ENT expression for modulating GEM responsiveness by Pin1i, established WT or ENT1-KD PDAC organoids were pre-treated with Pin1i−2 for 3 days, followed by treatment with GEM for 24 hrs. Pin1i−2 induced apoptosis in WT organoids, but not in ENT1-KD PDAC organoids (FIG. 13B and FIG. 13C), supporting the role of ENT1 in GEM response (Amrutkar and Gladhaug, Cancers (Basel) 9, 157 (2017); Farrell et al., Gastroenterology 136, 187-195 (2009)).

To examine the effects of Pin1 inhibition on the ability of ICB to induce PDAC organoid apoptosis, Pin1i pretreated human PDAC organoids were co-cultured with activated human primary peripheral blood mononuclear cells (PBMCs) that had been stimulated by human organoid cultured media and activated by anti-CD3 (αCD3) and anti-CD28 (αCD28) coated beads, and IL2, and then treated with αPD1 or αPD-L1, followed by time lapse apoptosis assay for 40 hrs (FIG. 6B), as described in (Dijkstra et al., Cell 174, 15861598 e1512 (2018); Jiao et al., Clin Cancer Res 23, 3711-3720 (2017)). Both Pin1 inhibitors increased the ability of αPD1 or αPD-L1 to induce organoid apoptosis, and the effects were again dose-dependent and highly synergistic (FIG. 6F-FIG. 6H, FIG. 13D, FIG. 13I, and FIG. 13J). To exclude possible off-target effects of Pin1 inhibitors, the same experiment was conducted with Pin1 KO and HIP1R929A PDAC organoids with activated human PBMCs and a similar enhancement of αPD1 anti-tumor effects was found (FIG. 12K). To demonstrate that the effects of Pin1 inhibition are dependent on PD-L1 expression in the PDAC organoid, WT or PD-L1-KD PDAC organoids were pre-treated with Pin1i−2 for 3 days, and then co-cultured with activated human PBMCs, followed by αPD1 treatment for 40 hrs. Pin1i−2 promoted the apoptosis of WT organoids, but not of PD-L1-KD PDAC organoids (FIG. 13E and FIG. 13F), supporting the role of PD-L1 in αPD1 response (Ansell et al., N Engl J Med 372, 311-319 (2015); Deng et al., J Clin Invest 124, 687-695 (2014); Galluzzi et al., Science translational medicine 10, eaat7807 (2018); Herbst et al., Nature 515, 563-567 (2014); Herter-Sprie et al., JCI Insight 1, e87415 (2016); Jiao et al., Clin Cancer Res 23, 3711-3720 (2017); Ruscetti et al., Cell 181, 424-441 e421 (2020); Zhang et al., Nature 553, 91-95 (2018)).

To further examine the ability of Pin1 inhibition to synergize with immunochemotherapy to induce PDAC organoid apoptosis, Pin1-inhibited organoids were incubated with activated PBMCs and with GEM+αPD1 (G+P) at reduced doses. Both Pin1 inhibitors time- and dose-dependently enhanced the ability of G+P to induce apoptosis of human PDAC organoids, and the effects were also highly synergistic (FIG. 6I-FIG. 6K, FIG. 13K, and FIG. 13L), as shown in GDA mice (FIG. 1E).

To use an autologous organoid model to confirm the above findings that CD8+ cytotoxic T-cells are important factor for the response of Pin1i+αPD1 therapy in GDA mice (FIG. 1H and FIG. 9E-FIG. 9G), KPC PDAC organoids were co-cultured with the same KPC tumor-bearing mice or their tumor-free littermate mice derived from CD8+ T-cells that have been activated by αCD3 and αCD28 coated beads, and IL2. As expected, the activated CD8+ T-cells derived from tumor-bearing mice were significantly more effective in inducing organoid apoptosis than those from non-tumor-bearing controls (FIG. 6L and FIG. 13M). More importantly, Pin1i synergized with αPD1 to induce PDAC organoid apoptosis (FIG. 6M and FIG. 6N), and these synergistic effects were also upheld when GEM was used (FIG. 13N and FIG. 13O), as in GDA mice (FIG. 1E), confirming the critical role of cytotoxic CD8+ T-cells for Pin1 inhibitors to potentiate immunotherapy response. Thus, targeting Pin1 acts on PDAC cells not only to inhibit multiple cancer pathways and induce the cell surface expression of PD-L1 and ENT1, but also to synergize with immunochemotherapy to induce apoptosis of human and mouse PDAC organoids.

### Example 8: Targeting Pin1 Renders Primary PDAC Tumors Eradicable by Immunochemotherapy in Genetically Engineered KPC Transgenic Mice

To determine whether Pin1 inhibitors are able to disrupt the highly desmoplastic and immunosuppressive TME and render primary PDAC tumors eradicable by immunochemotherapy in genetically engineered mouse mice, KPC transgenic mice were used because they express commonly occurring K-RasG12D and p53R172H mutations in the pancreas. These mice developed the spectrum of PDAC with 100% penetrance, which recapitulate tumor heterogeneity, desmoplastic and immunosuppressive TME, poor immunogenicity and rapid progression typical of human disease (Bayne et al., Cancer Cell 21, 822-835 (2012); Hingorani et al., Cancer Cell 7, 469-483 (2005)). Overt tumor-bearing KPC mice were treated with Pin1i or GEM+αPD1 (G+P) or their combination. Along with previous observations (Jiang et al., Nat Med 22, 851-860 (2016)), G+P neither affected the desmoplastic and immunosuppressive TME, nor increased survival, with the majority of the mice succumbing to the disease within 3 months (FIG. 7B and FIG. 7D-FIG. 7F). However, Pin1i−1 or Pin1i−2 and G+P combination suppressed cancer cell proliferation, and CAF activation and proliferation (FIG. 7A, FIG. 7C, FIG. 14A, and FIG. 14B), disrupted the desmoplastic and immunosuppressive TME, increased tumor infiltrating CD8α+Granzyme B+ CTLs (FIG. 7B, FIG. 7D and FIG. 7E) and induced PD-L1 and ENT1 expression in cancer cells (FIG. 14C and FIG. 14D). Importantly, Pin1i−1 or Pin1i−2 and G+P combination drastically increased survival, with 60-70% of treated mice surviving for at least 6 months after treatment (FIG. 7F). Upon necropsy, these surviving mice had no obvious macroscopic tumors, and only small microscopic tumors were notable (FIG. 7G and FIG. 14E), without liver metastases (FIG. 14F), indicating that tumors are disappearing despite continued expression of oncogenic mutant Kras and p53 in the pancreas. In conclusion, Pin1 inhibitors not only block multiple cancer pathways, disrupt the desmoplastic and immunosuppressive TME, and induce PD-L1 and ENT1 expression, but also render PDAC tumors eradicable by immunochemotherapy in GDA and KPC mice (FIG. 14G).

Table 3 is a table of shRNA sequence information related to STAR methods. The sequences set forth in Table 3 are consecutively numbered SEQ ID NO: 1-15.

TABLE 3
shRNA Sequence Infounation Related to STAR Medrods

| | Gene | | | | |
|---|---|---|---|---|---|
| PRODUCT | symbol (ID) | TRC Number | Clone ID | Sequence | SEQ ID NO |
| MISSION ® shRNA clone for | HIP1R | TRLN0000361009 | [symbol] | GTACCGGAGATGCTGTGCGGAGGATTGACTC | SEQ ID NO: 1 |
| human HIP1R (Sigma) | (9026) | | | GAGTCAATCCTCCGCACAGCATCTTTTTTTG | |
| MISSION ® shRNA clone for | HIP1R | TRLN0000361235 | [symbol] | GTACCGGTGTTCTCGCACAGTCAATGAGCTC | SEQ ID NO: 2 |
| human HIP1R (Sigma) | (9026) | | | GAGCTCATTGACTGTGCGAGAACATTTTTTG | |
| MISSION ® shRNA clone for | HIP1R | TRLN0000381579 | [symbol] | GTACCGGGGACAGAGACACCATGGATTTCTC | SEQ ID NO: 3 |
| human HIP1R (Sigma) | (9026) | | | GAGAAATCCATGGTGTCTCTGTGCTTTTTTG | |
| MISSION ® shRNA clone for | HIP1R | TRLN0000361959 | [symbol] | GTACCGGCAGAAGGCCCTGGTGGATAATCTC | SEQ ID NO: 4 |
| human HIP1R (Sigma) | (9026) | | | GAGATTATCCAGGAGGGCCTTGTGTTTTTTG | |
| MISSION ® shRNA clone for | HIP1R | TRLN0000382458 | [symbol] | GTACCGGACCTCTTGGATCAGACGTTTGCTC | SEQ ID NO: 5 |
| human HIP1R (Sigma) | (9026) | | | AGCAAACGTCTGATGGAAGAGGTTTTTTTTG | |
| MISSION ® shRNA clone for | SLC29A1 | TRCN0000043343 | [symbol] | CCGGGCTGTTATTCACCTACCTCAACTCGAG | SEQ ID NO: 6 |
| human SLC29A1 (ENT1)(Sigma) | (2030) | | | TTGAGGTAGGTGAATAACAGCTTTTTG | |
| MISSION ® shRNA clone for | SLC29A1 | TRCN0000043644 | [symbol] | CCGGCCACCAATGAAAGCCACTCTACTCGA | SEQ ID NO: 7 |
| human SLC29A1 (ENT1)(Sigma) | (2030) | | | GTAGAGTGGCTTTCATTGGTGGTTTTTG | |
| MISSION ® shRNA clone for | SLC29A1 | TRCN0000043345 | [symbol] | CCGGCGATGCCTGGTTCATCTTCTTCTCGAG | SEQ ID NO: 8 |
| human SLC29A1 (ENT1)(Sigma) | (2030) | | | AAGAAGATGAACCAGGCATCGTTTTTG | |
| MISSION ® shRNA clone for | SLC29A1 | TRCN0000043646 | [symbol] | CCGGCCTGGAATTCTACCGCTACTACTCGAG | SEQ ID NO: 9 |
| human SLC29A1 (ENT1)(Sigma) | (2030) | | | TACTACCCCTACAATTCCAGGTTTTTG | |
| MISSION ® shRNA clone for | SLC29A1 | TRCN0000043847 | [symbol] | CCGGCAAAGCTGTCTGGCTTATCTTCTCGAG | SEQ ID NO: 10 |
| human SLC29A1 (ENT1)(Sigma) | (2030) | | | AAGATAAGCCAGACAGCTTTGTTTTTG | |
| MISSION ® shRNA clone for | CD274 | TRCN0000056913 | [symbol] | CCGGGCACTAATTGTCTATTGGGAACTCGAG | SEQ ID NO: 11 |
| human CD274 (PD-L1)(Sigma) | (29126) | | | TTCCCAATAGACAATTAGTGCTTTTTG | |
| MISSION ® shRNA clone for | CD274 | TRCN0000056914 | [symbol] | CCGGCCAATTACTGTGAAAGTCAATCTCGAG | SEQ ID NO: 12 |
| human CD274 (PD-L1)(Sigma) | (29126) | | | ATTGACTTTCACAGTAATTCGTTTTTG | |
| MISSION ® shRNA clone for | CD274 | TRCN0000056915 | [symbol] | CCGGCTGACATTCATCTTCCGTTTACTCGAGT | SEQ ID NO: 13 |
| human CD274 (PD-L1)(Sigma) | (29126) | | | AAACGGAAGATGAATGTCAGTTTTTG | |
| MISSION ® shRNA clone for | CD274 | TRCN0000056916 | [symbol] | CCGGGACCTATATGTGGTAGAGTATCTCGAG | SEQ ID NO: 14 |
| human CD274 (PD-L1)(Sigma) | (29126) | | | ATACTCTACCACATATAGGTCTTTTTG | |
| MISSION ® shRNA clone for | CD274 | TRCN00000432418 | [symbol] | CCGGATGACAATTGAATGCAAATTCCTCGAG | SEQ ID NO: 15 |
| human CD274 (PD-L1)(Sigma) | (29126) | | | GAATTTGCATTCAATTGTCATTTTTTTG | |

[symbol]

indicates data missing or illegible when filed

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating a disease or disorder mediated by dysregulated Pin1 activity, in a subject in need thereof, comprising co-administering a therapeutically effective amount of one or more Pin1 inhibitors, or a pharmaceutically acceptable salt or salts thereof, and a therapeutically effective amount of an additional immunotherapy and/or chemotherapy.

2. A method of reducing the activity of Pin1 in a cell, either in vivo or in vitro, comprising co-administering a therapeutically effective amount of one or more Pin1 inhibitors, or a pharmaceutically acceptable salt or salts thereof, and a therapeutically effective amount of an additional immunotherapy and/or chemotherapy.

3. The method of claim 1, wherein the co-administering results in greater therapeutic effect than the effect of the additional immunotherapy and/or chemotherapy when administered alone as a sole active agent, without one or more Pin1 inhibitors.

4. The method of claim 1, wherein the one or more Pin1 inhibitors is all-trans retinoic acid (ATRA), arsenic trioxide (ATO), sulfopin, or a combination thereof, or a pharmaceutically acceptable salt or salts thereof.

5. The method of claim 1, wherein the one or more Pin1 inhibitors comprises ATRA and ATO (Pin1i−1), or wherein the one or more Pin1 inhibitors comprises sulfopin (Pin1i−2).

6. (canceled)

7. The method of claim 1, wherein the chemotherapy comprises gemcitabine (GEM) or fluorouracil (5-FU); or wherein the immunotherapy is anti-PD-1 or anti-PD-L1.

8. (canceled)

9. The method of claim 1, wherein the co-administering comprises Pin1i−1 and GEM; or
wherein the co-administering comprises Pin1i−2 and GEM; or
wherein the co-administering comprises Pin1i−1 and 5-FU; or
wherein the co-administering comprises Pin1i−2 and 5-FU; or
wherein the co-administering comprises Pin1i−1 and anti-PD-1; or
wherein the co-administering comprises Pin1i−2 and anti-PD-1; or
wherein the co-administering comprises Pin1i−1, anti-PD-1, and GEM; or
wherein the co-administering comprises Pin1i−2, anti-PD-1, and GEM.

10.-16. (canceled)

17. The method of claim 1, comprising pre-treatment with the one or more Pin1 inhibitors prior to the co-administering.

18. The method of claim 1, wherein the disease is cancer.

19. The method of claim 18, wherein the cancer is a solid tumor cancer.

20. The method of claim 19, wherein the solid tumor cancer is pancreatic ductal adenocarcinoma (PDAC), breast cancer, colorectal cancer, or acute promyelocytic leukemia.

21. The method of claim 19, wherein the solid tumor cancer is PDAC.

22.-23. (canceled)

24. The method of claim 18, comprising pre-treatment with the one or more Pin1 inhibitors prior to the co-administering.

25. A pharmaceutical composition, comprising a therapeutically effective amount of one or more Pin1 inhibitors, wherein the one or more Pin1 inhibitor is all-trans retinoic acid (ATRA), arsenic trioxide (ATO), sulfopin, or a combination thereof, or a pharmaceutically acceptable salt thereof, and a therapeutically effective amount of an additional immunotherapy and/or chemotherapy, which is in the form of a liquid or a solid.

26.-27. (canceled)

28. The pharmaceutical composition of claim 25, which is in the form of a tablet or capsule.

29. The pharmaceutical composition of claim 25, wherein the ATRA is in the form of a slow-release formulation.

30. The method of claim 2, wherein the co-administering results in greater therapeutic effect than the effect of the additional immunotherapy and/or chemotherapy when administered alone as a sole active agent, without one or more Pin1 inhibitors.

31. The method of claim 2, wherein the one or more Pin1 inhibitors is all-trans retinoic acid (ATRA), arsenic trioxide (ATO), sulfopin, or a combination thereof, or a pharmaceutically acceptable salt or salts thereof.

32. The method of claim 2, wherein the one or more Pin1 inhibitors comprises ATRA and ATO (Pin1i−1), or wherein the one or more Pin1 inhibitors comprises sulfopin (Pin1i−2).

33. The method of claim 2, wherein the chemotherapy comprises gemcitabine (GEM) or fluorouracil (5-FU); or wherein the immunotherapy is anti-PD-1 or anti-PD-L1.

34. The method of claim 2, wherein the co-administering comprises Pin1i−1 and GEM; or wherein the co-administering comprises Pin1i−2 and GEM; or wherein the co-administering comprises Pin1i−1 and 5-FU; or wherein the co-administering comprises Pin1i−2 and 5-FU; or wherein the co-administering comprises Pin1i−1 and anti-PD-1; or wherein the co-administering comprises Pin1i−2 and anti-PD-1; or wherein the co-administering comprises Pin1i−1, anti-PD-1, and GEM; or wherein the co-administering comprises Pin1i−2, anti-PD-1, and GEM.

35. The method of claim 2, comprising pre-treatment with the one or more Pin1 inhibitors prior to the co-administering.


SEQUENCE LISTING (37 sequences)

SEQ ID NO: 1 - DNA, 62 bp, Homo sapiens
gtaccggagatgctgtgcggaggattgactcgagtcaatcctccgcacagcatctttttttg

SEQ ID NO: 2 - DNA, 62 bp, Homo sapiens
gtaccggtgttctcgcacagtcaatgagctcgagctcattgactgtgcgagaacattttttg

SEQ ID NO: 3 - DNA, 62 bp, Homo sapiens
gtaccggggacagagacaccatggatttctcgagaaatccatggtgtctctgtccttttttg

SEQ ID NO: 4 - DNA, 62 bp, Homo sapiens
gtaccggcagaaggccctggtggataatctcgagattatccaccagggccttctgttttttg

SEQ ID NO: 5 - DNA, 62 bp, Homo sapiens
gtaccggacctcttcgatcagacgtttgctcgagcaaacgtctgatcgaagaggtttttttg

SEQ ID NO: 6 - DNA, 58 bp, Homo sapiens
ccgggctgttattcacctacctcaactcgagttgaggtaggtgaataacagctttttg

SEQ ID NO: 7 - DNA, 58 bp, Homo sapiens
ccggccaccaatgaaagccactctactcgagtagagtggctttcattggtggtttttg

SEQ ID NO: 8 - DNA, 58 bp, Homo sapiens
ccggcgatgcctggttcatcttcttctcgagaagaagatgaaccaggcatcgtttttg

SEQ ID NO: 9 - DNA, 58 bp, Homo sapiens
ccggcctggaattctaccgctactactcgagtagtagcggtagaattccaggtttttg

SEQ ID NO: 10 - DNA, 58 bp, Homo sapiens
ccggcaaagctgtctggcttatcttctcgagaagataagccagacagctttgtttttg

SEQ ID NO: 11 - DNA, 58 bp, Homo sapiens
ccgggcactaattgtctattgggaactcgagttcccaatagacaattagtgctttttg

SEQ ID NO: 12 - DNA, 58 bp, Homo sapiens
ccggcgaattactgtgaaagtcaatctcgagattgactttcacagtaattcgtttttg

SEQ ID NO: 13 - DNA, 58 bp, Homo sapiens
ccggctgacattcatcttccgtttactcgagtaaacggaagatgaatgtcagtttttg

SEQ ID NO: 14 - DNA, 58 bp, Homo sapiens
ccgggacctatatgtggtagagtatctcgagatactctaccacatataggtctttttg

SEQ ID NO: 15 - DNA, 59 bp, Homo sapiens
ccggatgacaattgaatgcaaattcctcgaggaatttgcattcaattgtcatttttttg

SEQ ID NO: 16 - DNA, 23 bp, Homo sapiens
agtcacggcggccctcgtcctgg

SEQ ID NO: 17 - DNA, 20 bp, Homo sapiens
cagtggtggcaaaaacgggc

SEQ ID NO: 18 - DNA, 17 bp, Homo sapiens
ccccccacctgagccgc

SEQ ID NO: 19 - DNA, 24 bp, Homo sapiens
cgtgcttgttggccttcaccttgg

SEQ ID NO: 20 - DNA, 20 bp, Homo sapiens
cccctggagaggaggtggcc

SEQ ID NO: 21 - DNA, 19 bp, Homo sapiens
cgcctgatgccccagccag

SEQ ID NO: 22 - DNA, 32 bp, Homo sapiens
atcatcctcgagccaccatgaacagcatcaag

SEQ ID NO: 23 - DNA, 39 bp, Homo sapiens
atcgcggccgctcacttatcgtcgtcatccttgtaatcg

SEQ ID NO: 24 - DNA, 22 bp, Homo sapiens
agcctcaagatcatcagcaatg

SEQ ID NO: 25 - DNA, 22 bp, Homo sapiens
tgatggcatggactgtggtcat

SEQ ID NO: 26 - DNA, 19 bp, Homo sapiens
gcctcacagttcagcgact

SEQ ID NO: 27 - DNA, 21 bp, Homo sapiens
actcagtgcggaggatgatgt

SEQ ID NO: 28 - DNA, 20 bp, Homo sapiens
cagaaagtgccttcggctac

SEQ ID NO: 29 - DNA, 22 bp, Homo sapiens
tgggctgagagagttggagact

SEQ ID NO: 30 - DNA, 21 bp, Homo sapiens
tggcatttgctgaacgcattt

SEQ ID NO: 31 - DNA, 22 bp, Homo sapiens
tgcagccaggtctaattgtttt

SEQ ID NO: 32 - DNA, 20 bp, Homo sapiens
ggtgccgactacaagcgaat

SEQ ID NO: 33 - DNA, 20 bp, Homo sapiens
agccctcagcctgacatgtc

SEQ ID NO: 34 - DNA, 20 bp, Homo sapiens
atttggaggatgtgccagag

SEQ ID NO: 35 - DNA, 21 bp, Homo sapiens
ccagcacactgagaatcaaca

SEQ ID NO: 36 - DNA, 24 bp, Homo sapiens
cctactggcatttgctgaacgcat

SEQ ID NO: 37 - DNA, 24 bp, Homo sapiens
accatagctgatcatgcagcggta